SEQUENCE LISTING (60 sequences)

SEQ ID NO: 1 - DNA, 19 bp, unidentified
  source 1..19: mol_type = other DNA
taatacgactcactatagg

SEQ ID NO: 2 - RNA, 130 nt, unidentified
  source 1..130: mol_type = mRNA
acaacagataacttacagctagtcggaaggtgcagagactcgacgggagctaccctaacgtcaagacgagggtaaagagagagtccaattctcaaagccaataggcagtagcgaaagctgcgggagaatg

SEQ ID NO: 3 - RNA, 51 nt, unidentified
  source 1..51: mol_type = mRNA
aaaatccgttgaccttaaacggtcgtgtgggttcaagtccctccaccccca

SEQ ID NO: 4 - RNA, 27 nt, unidentified
  source 1..27: mol_type = mRNA
tggcggaatggtagacgctacggactt

SEQ ID NO: 5 - RNA, 119 nt, unidentified
  source 1..119: mol_type = mRNA
aaataattgagccttagagaagaaattctttaagtggatgctctcaaactcagggaaacctaaatctagctatagacaaggcaatcctgagccaagccgaagtagtaattagtaagtta

SEQ ID NO: 6 - RNA, 450 nt, unidentified
  source 1..450: mol_type = mRNA
cccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattg

SEQ ID NO: 7 - RNA, 1249 nt, unidentified
  source 1..1249: mol_type = mRNA
cccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaactccttctccacaagcgccttcggtccagttgccttctccctgggcctgctcctggtgttgcctgctgccttccctgccccaggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcttgcaggacaggggacagatag

SEQ ID NO: 8 - RNA, 10 nt, unidentified
  source 1..10: mol_type = mRNA
tagacaccac

SEQ ID NO: 9 - RNA, 15 nt, unidentified
  source 1..15: mol_type = mRNA
tagacaccactacgg

SEQ ID NO: 10 - RNA, 19 nt, unidentified
  source 1..19: mol_type = mRNA
ccgtcgattgtccactggt

SEQ ID NO: 11 - RNA, 23 nt, unidentified
  source 1..23: mol_type = mRNA
aaataattgagccttagagaaga

SEQ ID NO: 12 - RNA, 98 nt, unidentified
  source 1..98: mol_type = mRNA
aattctttaagtggatgctctcaaactcagggaaacctaaatctagctatagacaaggcaatcctgagccaagccgaagtagtaattagtaagttaac

SEQ ID NO: 13 - RNA, 128 nt, unidentified
  source 1..128: mol_type = mRNA
aacagataacttacagctagtcggaaggtgcagagactcgacgggagctaccctaacgtcaagacgagggtaaagagagagtccaattctcaaagccaataggcagtagcgaaagctgcgggagaatg

SEQ ID NO: 14 - RNA, 70 nt, unidentified
  source 1..70: mol_type = mRNA
aaataattgagccttagagaagaaattctttaagtggatgctctcaaactcagggaaacctaaatctagc

SEQ ID NO: 15 - RNA, 51 nt, unidentified
  source 1..51: mol_type = mRNA
tatagacaaggcaatcctgagccaagccgaagtagtaattagtaagttaac

SEQ ID NO: 16 - RNA, 128 nt, unidentified; identical to SEQ ID NO: 13
  source 1..128: mol_type = mRNA

SEQ ID NO: 17 - RNA, 121 nt, unidentified
  source 1..121: mol_type = mRNA
aaataattgagccttagagaagaaattctttaagtggatgctctcaaactcagggaaacctaaatctagctatagacaaggcaatcctgagccaagccgaagtagtaattagtaagttaac

SEQ ID NO: 18 - RNA, 61 nt, unidentified
  source 1..61: mol_type = mRNA
aacagataacttacagctagtcggaaggtgcagagactcgacgggagctaccctaacgtca

SEQ ID NO: 19 - RNA, 67 nt, unidentified
  source 1..67: mol_type = mRNA
agacgagggtaaagagagagtccaattctcaaagccaataggcagtagcgaaagctgcgggagaatg

SEQ ID NO: 20 - RNA, 121 nt, unidentified; identical to SEQ ID NO: 17
  source 1..121: mol_type = mRNA

SEQ ID NO: 21 - RNA, 99 nt, unidentified
  source 1..99: mol_type = mRNA
aacagataacttacagctagtcggaaggtgcagagactcgacgggagctaccctaacgtcaagacgagggtaaagagagagtccaattctcaaagccaa

SEQ ID NO: 22 - RNA, 29 nt, unidentified
  source 1..29: mol_type = mRNA
taggcagtagcgaaagctgcgggagaatg

SEQ ID NO: 23 - RNA, 26 nt, unidentified
  source 1..26: mol_type = mRNA
tatagacaaggcaatcctgagccaag

SEQ ID NO: 24 - RNA, 153 nt, unidentified
  source 1..153: mol_type = mRNA
ccgaagtagtaattagtaagttaacaacagataacttacagctagtcggaaggtgcagagactcgacgggagctaccctaacgtcaagacgagggtaaagagagagtccaattctcaaagccaataggcagtagcgaaagctgcgggagaatg

SEQ ID NO: 25 - RNA, 35 nt, unidentified
  source 1..35: mol_type = mRNA
tatagacaaggcaatcctgagccaagccgaagccg

SEQ ID NO: 26 - RNA, 147 nt, unidentified
  source 1..147: mol_type = mRNA
tagtaattagtaagttaacaacagataacttacagctagtcggaaggtgcagagactcgacgggagctaccctaacgtcaagacgagggtaaagagagagtccaattctcaaagccaataggcagtagcgaaagctgcgggagaatg

SEQ ID NO: 27 - RNA, 38 nt, unidentified
  source 1..38: mol_type = mRNA
tatagacaaggcaatcctgagccaagccgaagtagtaa

SEQ ID NO: 28 - RNA, 141 nt, unidentified
  source 1..141: mol_type = mRNA
ttagtaagttaacaacagataacttacagctagtcggaaggtgcagagactcgacgggagctaccctaacgtcaagacgagggtaaagagagagtccaattctcaaagccaataggcagtagcgaaagctgcgggagaatg

SEQ ID NO: 29 - RNA, 41 nt, unidentified
  source 1..41: mol_type = mRNA
tatagacaaggcaatcctgagccaagccgaagtagtaatta

SEQ ID NO: 30 - RNA, 138 nt, unidentified
  source 1..138: mol_type = mRNA
gtaagttaacaacagataacttacagctagtcggaaggtgcagagactcgacgggagctaccctaacgtcaagacgagggtaaagagagagtccaattctcaaagccaataggcagtagcgaaagctgcgggagaatg

SEQ ID NO: 31 - RNA, 229 nt, unidentified
  source 1..229: mol_type = mRNA
aacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccatggcggaatggtagacgctacggacttaaataattgagccttagagaaga

SEQ ID NO: 32 - RNA, 276 nt, unidentified
  source 1..276: mol_type = mRNA
aacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccatggcggaatggtagacgctacggacttaaataattgagccttagagaagaaattctttaagtggatgctctcaaactcagggaaacctaaatctagc

SEQ ID NO: 33 - RNA, 266 nt, unidentified
  source 1..266: mol_type = mRNA
agacgagggtaaagagagagtccaattctcaaagccaataggcagtagcgaaagctgcgggagaatgaaaatccgttgaccttaaacggtcgtgtgggttcaagtccctccacccccatggcggaatggtagacgctacggacttaaataattgagccttagagaagaaattctttaagtggatgctctcaaactcagggaaacctaaatctagctatagacaaggcaatcctgagccaagccgaagtagtaattagtaagttaac

SEQ ID NO: 34 - RNA, 228 nt, unidentified
  source 1..228: mol_type = mRNA
taggcagtagcgaaagctgcgggagaatgaaaatccgttgaccttaaacggtcgtgtgggttcaagtccctccacccccatggcggaatggtagacgctacggacttaaataattgagccttagagaagaaattctttaagtggatgctctcaaactcagggaaacctaaatctagctatagacaaggcaatcctgagccaagccgaagtagtaattagtaagttaac

SEQ ID NO: 35 - RNA, 301 nt, unidentified
  source 1..301: mol_type = mRNA
ccgaagtagtaattagtaagttaacaacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccatggcggaatggtagacgctacggacttaaataattgagccttagagaagaaattctttaagtggatgctctcaaactcagggaaacctaaatctagc

SEQ ID NO: 36 - RNA, 295 nt, unidentified
  source 1..295: mol_type = mRNA
tagtaattagtaagttaacaacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccatggcggaatggtagacgctacggacttaaataattgagccttagagaagaaattctttaagtggatgctctcaaactcagggaaacctaaatctagc

SEQ ID NO: 37 - RNA, 289 nt, unidentified
  source 1..289: mol_type = mRNA
ttagtaagttaacaacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccatggcggaatggtagacgctacggacttaaataattgagccttagagaagaaattctttaagtggatgctctcaaactcagggaaacctaaatctagc

SEQ ID NO: 38 - RNA, 286 nt, unidentified
  source 1..286: mol_type = mRNA
gtaagttaacaacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccatggcggaatggtagacgctacggacttaaataattgagccttagagaagaaattctttaagtggatgctctcaaactcagggaaacctaaatctagc

SEQ ID NO: 39 - RNA, 78 nt, unidentified
  source 1..78: mol_type = mRNA
aaaatccgttgaccttaaacggtcgtgtgggttcaagtccctccacccccatggcggaatggtagacgctacggactt

SEQ ID NO: 40 - RNA, 726 nt, unidentified
  source 1..726: mol_type = mRNA
aacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtggcggaatggtagacgctacggacttaaataattgagccttagagaagaaattctttaagtggatgctctcaaactcagggaaacctaaatctagc

SEQ ID NO: 41 - RNA, 43 nt, unidentified
  source 1..43: mol_type = mRNA
gtggttatagacaaggcaatcctgagccaagccgaagtagtaa

SEQ ID NO: 42 - RNA, 59 nt, unidentified
  source 1..59: mol_type = mRNA
gtggtgtctatatagacaaggcaatcctgagccaagccgaagtagtaattagtaagtta

SEQ ID NO: 43 - RNA, 64 nt, unidentified
  source 1..64: mol_type = mRNA
ccgtagtggtgtctatatagacaaggcaatcctgagccaagccgaagtagtaattagtaagtta

SEQ ID NO: 44 - RNA, 69 nt, unidentified
  source 1..69: mol_type = mRNA
gagtaccgtagtggtgtctatatagacaaggcaatcctgagccaagccgaagtagtaattagtaagtta

SEQ ID NO: 45 - RNA, 99 nt, unidentified
  source 1..99: mol_type = mRNA
tggcagcacaggatgatggcacactgccgagagtaccgtagtggtgtctatatagacaaggcaatcctgagccaagccgaagtagtaattagtaagtta

SEQ ID NO: 46 - RNA, 731 nt, unidentified
  source 1..731: mol_type = mRNA
aacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtggcggaatggtagacgctacggacttaaataattgagccttagagaagaaattctttaagtggatgctctcaaactcagggaaacctaaatctagcaccac

SEQ ID NO: 47 - RNA, 736 nt, unidentified
  source 1..736: mol_type = mRNA
aacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtggcggaatggtagacgctacggacttaaataattgagccttagagaagaaattctttaagtggatgctctcaaactcagggaaacctaaatctagctagacaccac

SEQ ID NO: 48 - RNA, 741 nt, unidentified
  source 1..741: mol_type = mRNA
aacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtggcggaatggtagacgctacggacttaaataattgagccttagagaagaaattctttaagtggatgctctcaaactcagggaaacctaaatctagctagacaccactacgg

SEQ ID NO: 49 - RNA, 746 nt, unidentified
  source 1..746: mol_type = mRNA
aacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtggcggaatggtagacgctacggacttaaataattgagccttagagaagaaattctttaagtggatgctctcaaactcagggaaacctaaatctagctagacaccactacggtactc

SEQ ID NO: 50 - RNA, 776 nt, unidentified
  source 1..776: mol_type = mRNA
aacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtggcggaatggtagacgctacggacttaaataattgagccttagagaagaaattctttaagtggatgctctcaaactcagggaaacctaaatctagctagacaccactacggtactctcggcagtgtgccatcatcctgtgctgcca

SEQ ID NO: 51 - RNA, 583 nt, unidentified
  source 1..583: mol_type = mRNA
cccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 52 - RNA, 558 nt, unidentified
  source 1..558: mol_type = mRNA
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 53 - RNA, 1427 nt, unidentified
  source 1..1427: mol_type = mRNA
aacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaatggcggaatggtagacgctacggacttaaataattgagccttagagaagaaattctttaagtggatgctctcaaactcagggaaacctaaatctagctagacaccac

SEQ ID NO: 54 - RNA, 1653 nt, unidentified
  source 1..1653: mol_type = mRNA
atggaggacgccaagaacattaagaagggcccagccccattctaccccctggaagacggcactgccggcgagcagctgcacaaggccatgaaacggtatgcactggtgcccggcacaatcgccttcaccgacgctcatatcgaggtggatatcacttacgccgagtacttcgagatgagcgtccggctggctgaggccatgaagagatatggcctgaacaccaatcatcggatcgtggtgtgcagcgaaaacagcctgcagtttttcatgcccgtcctgggcgccctgttcatcggagtggccgtggcccccgccaatgatatttacaatgaacgggaactgctgaacagcatgggcattagccagccaactgtggtgttcgtgagcaaaaagggcctgcagaagatcctgaacgtgcagaagaaactgcccattatccagaagatcatcatcatggacagcaagaccgattatcaggggttccagagcatgtataccttcgtgacaagccatctgccccccggctttaatgagtatgattttgtgcccgaaagcttcgatagggataaaaccatcgccctgatcatgaacagcagcggctccaccgggctgcccaaaggcgtggcactgccccatcggacagcctgcgtgcggttctcccacgctcgggaccccatcttcgggaaccagatcatccccgacaccgccattctgagcgtggtgccctttcatcatggctttggcatgtttaccaccctgggctacctcatctgtggcttcagggtggtgctcatgtatcgctttgaggaagaactgtttctgcggagcctgcaggactacaagatccagagcgctctgctggtgcccaccctgtttagcttcttcgccaagagcaccctcattgataagtatgatctgagcaatctgcacgagatcgccagcggcggggcccccctgagcaaggaggtgggagaagccgtggccaaacggttccacctccctgggatcaggcagggctatggactgaccgaaaccaccagcgccatcctgatcacccccgaaggcgatgacaagcccggcgctgtgggcaaagtggtgcccttcttcgaggctaaggtggtggatctggacactggcaaaaccctgggggtgaaccagcggggcgagctgtgcgtgcgggggcctatgattatgagcggctacgtgaataaccccgaggcaaccaatgccctgatcgacaaagatggctggctgcactctggggacattgcctattgggacgaagacgagcacttcttcatcgtcgatcggctgaagagcctgatcaagtataaaggctaccaggtggcccccgccgaactggagagcattctgctgcagcaccccaacatctttgatgccggcgtggcaggcctgccagatgatgacgccggcgaactgcccgccgccgtggtggtgctggagcacggcaagaccatgaccgagaaagaaatcgtggactatgtggcctctcaggtgaccaccgccaagaagctgcggggcggggtggtgttcgtcgacgaggtcccaaagggcctgaccggcaagctggatgcccggaaaatccgggagatcctgatcaaggccaagaaaggcggcaagatcgccgtgtaa

SEQ ID NO: 55 - RNA, 2522 nt, unidentified
  source 1..2522: mol_type = mRNA
aacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggaggacgccaagaacattaagaagggcccagccccattctaccccctggaagacggcactgccggcgagcagctgcacaaggccatgaaacggtatgcactggtgcccggcacaatcgccttcaccgacgctcatatcgaggtggatatcacttacgccgagtacttcgagatgagcgtccggctggctgaggccatgaagagatatggcctgaacaccaatcatcggatcgtggtgtgcagcgaaaacagcctgcagtttttcatgcccgtcctgggcgccctgttcatcggagtggccgtggcccccgccaatgatatttacaatgaacgggaactgctgaacagcatgggcattagccagccaactgtggtgttcgtgagcaaaaagggcctgcagaagatcctgaacgtgcagaagaaactgcccattatccagaagatcatcatcatggacagcaagaccgattatcaggggttccagagcatgtataccttcgtgacaagccatctgccccccggctttaatgagtatgattttgtgcccgaaagcttcgatagggataaaaccatcgccctgatcatgaacagcagcggctccaccgggctgcccaaaggcgtggcactgccccatcggacagcctgcgtgcggttctcccacgctcgggaccccatcttcgggaaccagatcatccccgacaccgccattctgagcgtggtgccctttcatcatggctttggcatgtttaccaccctgggctacctcatctgtggcttcagggtggtgctcatgtatcgctttgaggaagaactgtttctgcggagcctgcaggactacaagatccagagcgctctgctggtgcccaccctgtttagcttcttcgccaagagcaccctcattgataagtatgatctgagcaatctgcacgagatcgccagcggcggggcccccctgagcaaggaggtgggagaagccgtggccaaacggttccacctccctgggatcaggcagggctatggactgaccgaaaccaccagcgccatcctgatcacccccgaaggcgatgacaagcccggcgctgtgggcaaagtggtgcccttcttcgaggctaaggtggtggatctggacactggcaaaaccctgggggtgaaccagcggggcgagctgtgcgtgcgggggcctatgattatgagcggctacgtgaataaccccgaggcaaccaatgccctgatcgacaaagatggctggctgcactctggggacattgcctattgggacgaagacgagcacttcttcatcgtcgatcggctgaagagcctgatcaagtataaaggctaccaggtggcccccgccgaactggagagcattctgctgcagcaccccaacatctttgatgccggcgtggcaggcctgccagatgatgacgccggcgaactgcccgccgccgtggtggtgctggagcacggcaagaccatgaccgagaaagaaatcgtggactatgtggcctctcaggtgaccaccgccaagaagctgcggggcggggtggtgttcgtcgacgaggtcccaaagggcctgaccggcaagctggatgcccggaaaatccgggagatcctgatcaaggccaagaaaggcggcaagatcgccgtgtaatggcggaatggtagacgctacggacttaaataattgagccttagagaagaaattctttaagtggatgctctcaaactcagggaaacctaaatctagctagacaccac

SEQ ID NO: 56 - RNA, 954 nt, unidentified
  source 1..954: mol_type = mRNA
atggtgagcaaaggagaagagctatttacaggcgtggtccccatcctcgtagaactggatggagacgtgaacggccacaaatttagcgtcagcggggagggcgaaggcgatgccacatacggcaagctgactttgaagtttatttgtaccactgggaaactgcctgtgccctggcccaccctggtcaccacactgacctatggggtgcagtgcttctcccggtaccccgatcacatgaagcagcacgacttcttcaaatccgctatgccagaaggatacgttcaagagcgaactatcttcttcaaggatgacggtaactacaagacaagggccgaagtcaagtttgagggcgatacactagtgaatcgcattgagctgaaaggcatagactttaaagaggatgggaacatcctggggcacaagctcgaatacaactacaactcccacaatgtgtacatcatggcagataagcagaagaacggtataaaggtgaactttaagatcagacataacattgaggatggttcagtgcaactcgcagaccattaccagcaaaacacaccaatcggagatggccccgttctgctgcccgataatcattacctgagtacacagagcgccctgtcaaaggacccaaatgaaaaacgtgatcacatggtgctcttagaatttgtgaccgccgctgggatcacactgggcatggatgagctctataaacgcagtagagatatctcccacggctttcctcccgccgtggcagcacaggatgatggcacactgccgatgtcctgcgctcaagagagcgggatggatcggcatcccgccgcttgtgcttctgcgcggataaacgtaggatccgccgctaaaaggccggcggccacgaaaaaggccggccaggcaaaaaagaaaaagctggacggcagcggaagccccaagaagaagaggaaggtataa

SEQ ID NO: 57 - RNA, 1823 nt, unidentified
  source 1..1823: mol_type = mRNA
aacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccacccccacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaaaggagaagagctatttacaggcgtggtccccatcctcgtagaactggatggagacgtgaacggccacaaatttagcgtcagcggggagggcgaaggcgatgccacatacggcaagctgactttgaagtttatttgtaccactgggaaactgcctgtgccctggcccaccctggtcaccacactgacctatggggtgcagtgcttctcccggtaccccgatcacatgaagcagcacgacttcttcaaatccgctatgccagaaggatacgttcaagagcgaactatcttcttcaaggatgacggtaactacaagacaagggccgaagtcaagtttgagggcgatacactagtgaatcgcattgagctgaaaggcatagactttaaagaggatgggaacatcctggggcacaagctcgaatacaactacaactcccacaatgtgtacatcatggcagataagcagaagaacggtataaaggtgaactttaagatcagacataacattgaggatggttcagtgcaactcgcagaccattaccagcaaaacacaccaatcggagatggccccgttctgctgcccgataatcattacctgagtacacagagcgccctgtcaaaggacccaaatgaaaaacgtgatcacatggtgctcttagaatttgtgaccgccgctgggatcacactgggcatggatgagctctataaacgcagtagagatatctcccacggctttcctcccgccgtggcagcacaggatgatggcacactgccgatgtcctgcgctcaagagagcgggatggatcggcatcccgccgcttgtgcttctgcgcggataaacgtaggatccgccgctaaaaggccggcggccacgaaaaaggccggccaggcaaaaaagaaaaagctggacggcagcggaagccccaagaagaagaggaaggtataatggcggaatggtagacgctacggacttaaataattgagccttagagaagaaattctttaagtggatgctctcaaactcagggaaacctaaatctagctagacaccac

SEQ ID NO: 58 - RNA, 315 nt, unidentified
  source 1..315: mol_type = mRNA
agacgctacggacttaaataattgagccttagagaagaaattctttaagtggatgctctcaaactcagggaaacctaaatctagctatagacaaggcaatcctgagccaagccgaagtagtaattagtaagttaacaacagataacttacagctagtcggaaggtgcagagactcgacgggagctaccctaacgtcaagacgagggtaaagagagagtccaattctcaaagccaataggcagtagcgaaagctgcgggagaatgaaaatccgttgaccttaaacggtcgtgtgggttcaagtccctccaccccca

SEQ ID NO: 59-60 - skipped (2 entries)

(19) United States
(12) Patent Application Publication
(10) Pub. No.: US 2026/0286358 A1
(43) Pub. Date: Sep. 24, 2026

(54) CIRCULAR RNA AND PREPARATION METHODS THEREOF

(71) Applicant: BeOne Medicines I GmbH, Basel (CH)

(72) Inventors: Jiao LIN, Shanghai (CN); Yu LU, Shanghai (CN)

(73) Assignee: BeOne Medicines I GmbH, Basel (CH)

(21) Appl. No.: 19/559,285

(22) Filed: Mar. 6, 2026

Related U.S. Application Data
(63) Continuation of application No. PCT/IB2024/058665, filed on Sep. 5, 2024.

(30) Foreign Application Priority Data
Sep. 8, 2023 (WO) PCT/CN2023/117650

(51) Int. Cl. C12N 15/113

(52) U.S. Cl. CPC C12N 15/113; C12N 2310/122; C12N 2310/124; C12N 2310/531

(57) ABSTRACT

Compositions and methods for efficiently constructing circular RNA are described. In particular, described are trans-splicing methods involving multiple fragments of Group I intron or derivatives thereof for efficient production of circular RNA with substantially no side products of dimer or concatemer. Also described are compositions related to the methods.

### CROSS REFERENCE TO RELATED APPLICATIONS

This application is a continuation of International Patent Application No. PCT/IB2024/058665, filed Sep. 5, 2024, which claims priority from International Patent Application No. PCT/CN2023/117650, filed Sep. 8, 2023. The contents of these applications are incorporated herein by reference in their entirety.

### TECHNICAL FIELD

The present application relates to compositions and methods for efficiently constructing circular RNA via a trans-splicing reaction.

### SEQUENCE LISTING

The present application contains a sequence listing which has been submitted electronically in xml format, and is hereby incorporated by reference in its entirety. Said xml file was created on Mar. 6, 2026, is 71,134 bytes in size, and named 138881_1562_Sequence_Listing.xml.

### BACKGROUND

The mRNA-based COVID-19 vaccines and the recent clinical success of mRNA-based personalized cancer vaccines have fueled the hope and passion in developing mRNA-based therapeutics. Any protein can be expressed by mRNA via cell's translation machinery. The mRNA does not enter the nucleus. Thus, mRNA has no potential risk of altering the DNA in the nucleus to result in mutagenesis. Also, the production of mRNA, including large scale mRNA production, has become more efficient and faster. However, the instability of mRNA in vivo dampens the mRNA's translation efficiency, and the immunogenicity of mRNA also impedes the pace of the development of mRNA-based therapeutics. Although using modified nucleotides (e.g., 1 mψ) may reduce the immunogenicity and increase the stability and translation efficiency of mRNA, there remains a need to explore other options for RNA-based therapy.

Circular RNAs (circRNAs) are a class of single-stranded RNAs distinguished by their covalently closed topology. The unique structure of circRNAs gives them greater stability, longer half-life, and greater RNase R resistance than the linear mRNAs. CircRNAs are ubiquitous in species ranging from viruses to mammals and have numerous biological functions, including but not limited to, acting as miRNA sponges and protein sponges, and interacting with many different RNA-binding proteins (RBPs). A subset of circRNAs can also undergo cap-independent translation. It has been shown that circRNAs with internal ribosome entry sites (IRES) could be translated in vivo and in vitro. In addition, the immunogenicity of circRNAs is reported to be less than that of unmodified mRNA and comparable to that of modified mRNA (Molecular cell, 2019, 74, 508-520.).

CircRNAs with specific properties can be produced in vivo and in vitro (Obi et al., Methods, 2021, 196: 85-103). A circRNA can be produced in vitro by synthesizing a linear RNA precursor followed by ligating its ends to form a covalently-closed loop. There are several methods for the in vitro synthesis of circRNAs, including but not limited to, chemical ligation, T4 RNA/DNA ligase-mediated enzymatic ligation, and ribozymatic methods using self-splicing introns. Chemical ligation has relatively low ligated efficiency and needs to use cyanogen bromide (BrCN), which may have biosafety concerns. Enzymatic ligation is not desirable for the ligation of large RNA molecules, e.g., it may have intermolecular end-joining side reaction and it can be technically challenging to scale up. Ribozymatic methods using self-splicing introns are currently the most popular and widely used method in synthesizing circRNAs.

Self-splicing introns act as ribozymes and catalyze their own excision from the pre-mRNA. Naturally occurring Group I introns are characterized by a linear array of sequences (e.g., from the 5′-end to the 3′-end: 5′ exon-5′-intron-3′-intron-3′ exon) and structural features. The Group I introns are self-spliced by a two-step transesterification mechanism to ligate the 5′-exon and 3′ exon to form ligated linear exons. Permuting the introns to the following order 3′-intron-3′ exon-5′ exon-5′-intron, the introns can undergo cleavage and ligation reactions to form circular intronic RNA. This ribozyme-catalyzed approach, termed the permuted intron exon self-splicing system, or PIE system, e.g., derived from Anabaena tRNA intron and the thymidylate synthase (Td) gene of the T4 phage, has been reported for the formation of circular RNAs in vitro only with the assistance of GTP and Mg2+. A limitation of PIE system is that it is difficult to realize long RNA circularization. The PIE can be improved with the help of strong homology-arms. The modified RNA molecule used to synthesize circRNAs has the following components ordered from the 5′ end to 3′ end: 5′-homology arm-3′-intron-spacer-3′ exon-target RNA sequence-5′ exon-spacer-5′-intron-3′-homology arm, in which the 5′-homology arm is completely paired with 3′-homology arm. Although the improved PIE can efficiently circularize RNA molecule, the side products of improved PIE, e.g., dimer or concatemer resulting from the homology arms, may impair RNA circularization efficiency and bring difficulties to chemistry, manufacturing and control (CMC) production.

Therefore, there is still a need in the art for improved circular RNAs preparation methods.

### SUMMARY OF THE INVENTION

Inventors of the present application surprisingly found that a trans-splicing method involving multiple fragments of Group I intron or derivatives thereof can be used for efficient production of circular RNA, with substantially no side products, such as dimer or concatemer.

In one general aspect, the application relates to a pair of RNA molecules for making a circular RNA, comprising:
1) a first RNA molecule comprising the following elements operably connected and arranged in the order from the 5′-end to the 3′-end of the molecule:
   i) a 3′ intron sequence of a Group I intron,
   ii) a 3′ splice site of the Group I intron,
   iii) optionally, a 3′ exon sequence of the Group I intron,
   iv) an RNA sequence of interest,
   v) optionally, a 5′ exon sequence of the Group I intron,
   vi) a 5′ splice site of the Group I intron, and
   vii) a 5′ intron sequence of the Group I intron, and
(2) a second RNA molecule comprising an intermediate intron sequence of the Group I intron,
wherein when the first RNA molecule is contacted with the second RNA molecule, a scaffolding domain and a catalytic domain of the Group I intron are formed to thereby allow the production of the circular RNA comprising the 3′ exon sequence, the RNA sequence of interest and the 5′ exon sequence.

In certain embodiments, the 3′ intron sequence comprises the R sequence and the S sequence of the Group I intron, the 5′ intron sequence comprises the internal guide sequence (IGS) and the P sequence of the Group I intron, and the intermediate intron sequence comprises the Q sequence of the Group I intron.

In certain embodiment, the first RNA molecule comprises, from the 5′-end to the 3′-end of the molecule,
a) Fragment 3 comprising the 3′ intron sequence and the 3′ splice site operably connected and arranged in the order from the 5′-end to the 3′-end of Fragment 3;
b) optionally, a 3′ exon sequence;
c) the RNA sequence of interest;
d) optionally, a 5′ exon sequence;
e) Fragment 1 comprising the 5′ splice site and the 5′ intron sequence operably connected and arranged in the order from the 5′-end to the 3′-end of Fragment 1; and
the second RNA molecule comprises Fragment 2 having the intermediate intron sequence.

Preferably, Fragments 1-3 comprise the nucleotide sequences or variants thereof of three portions of the Group I intron obtained by dividing the intron with two interrupted sites, respectively. More preferably, the three portions of the Group I intron are obtained by dividing the intron with an interrupted site located within a loop of the P6 stem-loop region of the Group I intron, and another interrupted site located within a loop of the P2, P5, P8, or P9 stem-loop region of the Group I intron.

In some embodiment, the nucleotide sequence of interest comprises at least one protein-coding sequence and an internal ribosome entry site (IRES) operably linked thereto. In some embodiment, the nucleotide sequence of interest comprises a non-protein coding sequence.

In certain embodiment, one or more of the 3′ intron sequence, the 3′ splice site, the 3′ exon sequence, the 5′ exon sequence, the 5′ splice site, the 5′ intron sequence, the intermediate intron sequence useful for the invention described herein comprise or are derived from a corresponding native sequence of a Group I intron. In certain embodiments, sequences of one or more of the components useful for the invention are 100% identical to the corresponding native sequences. In other embodiments, sequences of the one or more components useful for the invention are modified sequences having at least 50%, at least 60%, at least 70%, at least 80%, at least 90%, at least 91%, at least 92%, at least 93%, at least 94%, at least 95%, at least 96%, at least 97%, at least 98%, or at least 99% sequence identity to the corresponding native sequences. The modified sequences can have 1, 2, 3, 4, 5, 6, 7, 8, 9, or 10 or more nucleotide substitutions, deletions or additions compared to the native sequence. The native sequences can be from any suitable Group I intron. Examples of suitable Group I introns include, but are not limited to, a Group I intron of an organism selected from the group consisting of a cyanobacterium Anabaena, T4 phage, Azoarcus sp. BH72, Enterobacteriophage T4, Bacteriophage Twort, Bacteriophage SPO1, Bacteriophage S3b, Bacillus anthracis, Clostridium botulinum, Tetrahymena thermophila, Dunaliella parva, Pneumocystis carinii, Physarum polycephalum, Anabaena sp. PCC7120, Scytonema hofmanni, Agrobacterium tumefaciens, Synechocystis PCC 6803, Synechococcus elongatus PCC 6301, Neurospora crassa, Candida albicans, Scytalidium cerradiumydiaces, Pediadiaces Chlamydomonas nivalis, Chlorella vulgaris, Amoebidium parasiticum, Neurospora crassa, Emericella nidulans, Saccharomyces cerevisiae, Schizosaccharomyces pombe, Neochloris aquatica, Dunaliella parva, Symkania negevensis, and Emericella nidulans. In certain embodiment, the native sequences are from a Group I intron from cyanobacterium Anabaena, such as the Group I intron of Cyanobacterium Anabaena sp. pre-tRNA-Leu gene. In certain other embodiments, the Group I intron is from a T4 phage, or Azoarcus sp. BH72. Preferably, the Group I intron is from a Cyanobacterium Anabaena sp. pre-tRNA-Leu gene, a td gene of T4 phage, or a pre-tRNA-Ile gene of Azoarcus sp. BH72.

In certain embodiments, the first RNA molecule comprises a 3′ exon sequence or a 5′ exon sequence. In other embodiment, the first RNA molecule comprises a 3′ exon sequence and a 5′ exon sequence. In certain embodiments, the first RNA molecule does not comprise a 3′ exon sequence or a 5′ exon sequence. In certain embodiments, the first RNA molecule does not comprise a 3′ exon sequence and a 5′ exon sequence.

In certain embodiments, the first and second RNA molecules can include additional features to optimize the production of circRNA. For example, the first RNA molecule can further comprise a 3′-end binding motif, and the second RNA molecule can further comprise a 5′-end binding motif, wherein the 3′-end binding motif and the 5′-end binding motif are capable of complementary pairing to form a double-stranded region. Preferably, the 3′-end binding motif and the 5′-end binding motif are perfectly complimentary to each other.

Another general aspect of the present application relates to a nucleic acid vector for making a circular RNA molecule. In particular, a nucleic acid vector encoding the first and/or second RNA molecules according to embodiments of the application. In preferred embodiments, the vector further comprises an RNA polymerase promoter sequence operably linked to the coding sequence of the RNA molecule(s). In some preferred embodiments, the promoter is selected form a T7 RNA polymerase promoter, a T6 viral RNA polymerase promoter, a SP6 viral RNA polymerase promoter, a T3 viral RNA polymerase promoter, or a T4 viral RNA polymerase promoter.

In another aspect, the present application provides a method for preparing a circular RNA, which comprises:
i) providing or obtaining a pair of RNA molecules according to an embodiment of the application;
ii) adding a buffer solution to the pair of RNA molecules to obtain a reaction mixture to allow the formation of the scaffolding domain and the catalytic domain of the Group I intron, and
iii) adding GTP and a divalent metal cation to the reaction mixture at a temperature to allow the formation of the circular RNA.
   Optionally, the method further comprises iv) harvesting the circular RNA formed in step iii).

In another aspects, the application provides a circular RNA produced by a method of the application. The application provides a composition comprising an effective amount of a circular RNA of the present application and a pharmaceutically acceptable carrier. Preferably, the pharmaceutically acceptable carrier comprises a lipid, a polymer or a lipo-polymeric hybrid, such as a lipid nanoparticle (LNP), a lipid microparticle, a lipid suspension, or a liposome.

Other aspects of the application include a composition comprising a cell, such as a eukaryotic cell, comprising a nucleic acid encoding a circular RNA or a circular RNA according to embodiments of the application. Also provided is a composition comprising the cell and one or more pharmaceutically or physiologically acceptable carriers, excipients or diluents.

Other aspects, features and advantages of the invention will be apparent from the following disclosure, including the detailed description of the invention and its preferred embodiments and the appended claims.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings.

FIG. 1. The schematic secondary structure of the Group I intron from Anabaena pre-tRNA.

FIG. 2. The scheme of classical Permuted-Intron-Exon (PIE) synthesis of a circular exon.

FIG. 3A. The scheme of classical PIE strategy for generating circRNAs with the RNA sequence of interest; FIG. 3B. The circRNA was verified by 5% denatured TBE-PAGE.

FIG. 4A. The scheme of improved PIE strategy for generating circRNAs with the RNA sequence of interest; FIG. 4B. The circularization with 10/15/19 bp-homology arms under two-circularizing conditions (A and B) was confirmed by 5% denatured UREA-TBE PAGE; and FIG. 4C. The long RNA circularization with 19 bp-homology arms under the B circularizing condition was confirmed by 2% E-gel EX.

FIGS. 5A-5D. The structures of multi-fragment active Anabaena pre-tRNA Group I introns.

FIG. 6. The self-splicing activity of muti-fragment Group I introns was verified by 20% denatured UREA-TBE PAGE. The first line is the fragment 1(F1) of Assembled intron-1, the second line is the fragment 2 (F2) of Assembled intron-1, the third line is the fragment 3 (F3) of Assembled intron-1, the fourth line is the mixture of F1, F2 and F3, meanwhile, muti-fragment intron-1 assembled by F1, F2 and F3 was self-splicing to produce linear exon (17 nt) indicated by red arrow; The fifth line is the fragment 1(F1) of Assembled intron-4, the sixth line is the fragment 2 (F2) of Assembled intron-4, the seventh line is the fragment 3 (F3) of Assembled intron-4, the eighth line is the mixture of F1, F2 and F3, meanwhile, muti-fragment intron-4 assembled by F1, F2 and F3 was self-splicing to produce linear exon (17 nt) indicated by red arrow.

FIG. 7. The splicing mechanism of multi-fragment active Anabaena pre-tRNA Group I intron.

FIGS. 8A-8D. The different split sites on P6 of muti-fragment active Anabaena pre-tRNA Group I intron.

FIG. 9. The scheme of trans-splicing strategy for synthesizing circular exon.

FIGS. 10A-10C. The circ-exon synthesized by trans-splicing strategy was verified by 5% UREA-TBE PAGE. FIG. 10A indicated the circ-exon marked by red arrow generated by P-AI-1 with the help of its corresponding F2, P-AI-1 and it corresponding F2 both derived from Assembled intron-1. FIG. 10B indicated the circ-exon marked by red arrow generated by P-AI-2 with the help of its corresponding F2, P-AI-2 and it corresponding F2 both derived from Assembled intron-2. FIG. 10C indicated the circ-exon marked by red arrow generated by P-AI-4 with the help of its corresponding F2, P-AI-4 and it corresponding F2 both derived from Assembled intron-4.

FIG. 11. The effect of split site of P6 on the circ-exon generated by trans-splicing strategy was verified by 5% UREA-TBE PAGE. The circ-exon was marked by red arrow.

FIG. 12A. The scheme of trans-splicing strategy and RNA splicing efficiency for synthesizing circRNAs with RNA sequence of interest; FIG. 12B. The circularization was confirmed by 5% denatured UREA-TBE PAGE.

FIG. 13A. The scheme of generating circRNA by trans-splicing strategy with various length binding motifs (BM). FIG. 13B. The length of binding motif (BM) affected the circularization efficiency of trans-splicing strategy was confirmed by 5% denatured UREA-TBE PAGE. Line 1 (\textcircled{1}) is the I(5)-P-RNA-seq 1, Line 2 (\textcircled{2}) is the mixture of I(5)-P-RNA-seq 1 and BM(5)-F2, meanwhile, circRNA marked by red circle was synthesized by I(5)-P-RNA-seq 1 with the help of BM(5)-F2; Line 3 (3) is the I(15)-P-RNA-seq 1, Line 4 (\textcircled{4}) is the mixture of I(15)-P-RNA-seq 1 and BM(10)-F2, meanwhile, circRNA marked by red circle was synthesized by I(15)-P-RNA-seq 1 with the help of BM(10)-F2; Line 5 (\textcircled{5}) is the mixture of I(15)-P-RNA-seq 1 and BM(15)-F2, meanwhile, circRNA marked by red circle was synthesized by I(15)-P-RNA-seq 1 with the help of BM(15)-F2; Line 6 (\textcircled{6}) is the I(20)-P-RNA-seq 1, Line 7 (\textcircled{7}) is the mixture of I(20)-P-RNA-seq 1 and BM(20)-F2, meanwhile, circRNA marked by red circle was synthesized by I(20)-P-RNA-seq 1 with the help of BM(20)-F2; Line 8 (\textcircled{8}) is the I(50)-P-RNA-seq 1, Line 7 (\textcircled{9}) is the mixture of I(50)-P-RNA-seq 1 and BM(50)-F2, meanwhile, circRNA marked by red circle was synthesized by I(20)-P-RNA-seq 1 with the help of BM(50)-F2;

FIG. 14A. The scheme of improved trans-splicing strategy and RNA splicing efficiency for synthesizing circRNAs; FIG. 14B. The circularization was verified by 5% denatured UREA-TBE PAGE; FIG. 14C. The circRNAs was confirmed by RT-PCR using the divergent primer which is across circRNA's junction site; and FIG. 14D. The circRNAs were confirmed by Sanger sequencing of the junction region.

FIG. 15. The improved trans-splicing strategy synthesizing circRNAs expressed secreted protein Gaussia Luciferase 293T cells.

FIG. 16. The improved trans-splicing strategy synthesizing circRNAs expressed intercellular protein firefly luciferase in 293T cells.

FIG. 17. The improved trans-splicing strategy synthesizing circRNAs expressed nuclear protein eEGFP-NLS in 293T cells.

### DETAILED DESCRIPTION OF THE INVENTION

Various publications, articles, patents and patent applications are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

In the present application, unless indicated otherwise, the scientific and technological terminologies used herein refer to meanings commonly understood by a person skilled in the art to which this invention pertains. Also, the terminologies and experimental procedures used herein relating to protein and nucleotide chemistry, molecular biology, cell and tissue cultivation, microbiology, immunology, all belong to terminologies and conventional methods generally used in the art. For example, the standard DNA recombination and molecular cloning technology used herein are well known to a person skilled in the art and are described in detail in the following references: Sambrook, J., Fritsch, Efland Maniatis, T, Molecular Cloning: A Laboratory Manual; Cold Spring Harbor Laboratory Press: Cold Spring Harbor, 1989. In the meantime, in order to better understand the present application, definitions and explanations for the relevant terminologies are provided below.

For the sake of clarity and readability, the following definitions are provided. Any technical feature mentioned for these definitions can be read on each and every embodiment of the application. Additional definitions and explanations may be specifically provided in the context of these embodiments.

“Polynucleotide”, “nucleic acid sequence”, “nucleotide sequence”, and “nucleic acid fragment” are used interchangeably to refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide. Although the nucleotide sequences herein may be represented as DNA sequences (comprising T(s)), when referring to RNA, one skilled in the art can readily determine the corresponding RNA sequence (i.e., replacing T with U).

The terms “circular RNA”, “circular polyribonucleotide”, and “circRNA” are used interchangeably and refer to a polyribonucleotide that forms a circular structure. Preferably, the circular structure is formed through covalent bonds.

The term “fragment” as used throughout the application in the context of a nucleic acid sequence or an amino acid sequence refers to a portion of a full-length sequence of the nucleic acid sequence or the amino add sequence. Accordingly, a fragment typically consists of a sequence that is identical to the corresponding stretch within the full-length sequence. A preferred fragment of a sequence in the context of the application, consists of a continuous stretch of entities, such as nucleotides or amino acids, corresponding to a continuous stretch of entities in the molecule the fragment is derived from, which represents at least 40%, 50%, 60%, 70%, 80%, 90%, 95% of the total (i.e., full-length) molecule from which the fragment is derived. The term “fragment’ as used throughout the present application, typically, comprises a sequence of a self-splicing intron as defined herein, which is, with regard to its nucleic acid sequence, 5′-terminally and/or 3′-terminally truncated compared to the nucleic acid sequence of the original intron. Such truncation can thus occur on the nucleic acid level.

A sequence identity with respect to such a fragment as defined herein can therefore preferably refer to the entire nucleic acid molecule.

The term “identity” has recognized meaning in the art, and the percentage of sequence identity between two nucleic acids or polypeptide molecules or regions can be calculated using techniques known in the art. Sequence identity can be measured along the entire length of a polynucleotide or polypeptide or along a region of the molecule. (See, for example, Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). There are many methods for determining sequence identity. An example of algorithms suitable for determining percent sequence identity is the algorithm used in the Basic Local Alignment Search Tool (hereinafter “BLAST”), see e.g., Altschul et al., J. Mol. Biol. 215:403-410,1990 and Altschul et al, Nucleic Acids Res., 15:3389-3402, 1997. Software for performing BLAST analysis is publicly available through the National Center for Biotechnology Information (hereafter “NCBI”). Default parameters used to determine sequence identity using software available from NCB (such as BLASTN for nucleic acid sequences) are described in McGinnis et al. Nucleic Acids Res., 32: W20-W25, 2004.

The terms “coding sequence”, “coding region” and the corresponding abbreviation “cds” as used herein each refer to a sequence containing multiple nucleotide triplets, which can be translated into a peptide or polypeptide. A coding sequence in the context of the present application can be an RNA sequence containing a number of nucleotides that can be divided by three, which starts with a start codon, and which preferably terminates with a stop codon.

The term “derived from” as used throughout the application in the context of a nucleic acid, i.e. for a nucleic acid “derived from” (another) nucleic acid, means that the nucleic acid, which is derived from (another) nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to a nucleic acid, which is modified in comparison to the nucleic acid from which it is derived, e.g. in order to increase RNA stability even further and/or to prolong and/or increase protein production. In the context of amino acid sequences (e.g. antigenic peptides or proteins) the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence from which it is derived.

The term “permute” as used herein in the context of a nucleic acid molecule means to change the sequence of the molecule. In the present application, a permuted molecule is sometimes equivalent to a precursor (represented by the prefix pre-in comparative examples, or by the prefix of P- in examples of the present application) of the molecule.

As used herein, the term “and/or” encompasses all combinations of items connected by the term, and each combination should be regarded as individually listed herein. For example, “A and/or B” covers “A”, “A and B”, and “B”. For example, “A, B, and/or C” covers “A”, “B”, “C”, “A and B”, “A and C”, “B and C”, and “A and B and C”.

Where reference is made to “SEQ ID NOs” of other patent applications or patents, said sequences, e.g., amino acid sequences or nucleic acid sequences, are explicitly incorporated herein by reference. For “SEQ ID NOs” provided herein, information under identifier < > (in the sequence protocol) is also explicitly included herein in its entirety. Where reference is made to “SEQ ID NOs” in the context of RNA sequences, the skilled person will understand and be able to derive RNA sequences from the referenced SEQ ID NOs also in cases where DNA sequences are provided. Where reference is made to “SEQ ID NOs” in the context of DNA sequences, the skilled person will understand and will be able to derive respective DNA sequences from the referenced SEQ ID NOs also in cases where RNA sequences are provided.

As used herein, the term “Group I intron” refers to a structured self-splicing intron that acts as a ribozyme and autocatalyzes its removal (splicing) from a primary transcript. Group I introns catalyze their own excision from mRNA, tRNA and rRNA precursors in a wide range of organisms. Group I introns contain 14 subgroups, while most of the Group I introns belong to the IC3 subgroup. For example, the Group I intron can be a Group I intron of the cyanobacterium Anabaena belonging to the IC3 subgroup or a Group I intron from a T4 phage Group I intron belonging to the IA2 subgroup or a Group I intron from Azoarcus sp. BH72 belonging to the IC3 subgroup. Additional examples of self-splicing introns useful in the present application include, but are not limited to, self-splicing introns derived from the following organisms: Enterobacteriophage T4, Bacteriophage Twort, Bacteriophage SPO1, Bacteriophage S3b, Bacillus anthracis, Clostridium botulinum, Tetrahymena thermophila, Dunaliella parva, Pneumocystis carinii, Physarum polycephalum, Anabaena sp. PCC7120, Scytonema hofmanni, Agrobacterium tumefaciens, Synechocystis PCC 6803, Synechococcus elongatus PCC 6301, Neurospora crassa, Candida albicans, Scytalidium cerradiumydiaces, Pediadiaces Chlamydomonas nivalis, Chlorella vulgaris, Amoebidium parasiticum, Neurospora crassa, Emericella nidulans, Saccharomyces cerevisiae, Schizosaccharomyces pombe, Neochloris aquatica, Dunaliella parva, Symkania negevensis, Emericella nidulans.

Many Group I introns can self-splice in vitro without assistance from protein co-factors. While Group I introns are highly variable at the primary sequence level, they share characteristic conserved secondary and tertiary structures. As illustrated in FIG. 1, Group I introns contain the characteristic secondary structure of paired (P) elements, e.g., P1 to P9, and single stranded loop regions, as defined by Waring and Davies (Davies, R W., Waring, R B., Ray, R A., Brown, T. A. and ca o, C. (1982) Nature 300, 719-724.). They are numbered according to their occurrence as one traces the RNA chain from 5′ to 3′. Note that P2 is the stem closest to the 3′ side of P1, and P9 is the stem closest to the 3′ side of P7.

Short conserved sequences named P, Q, R, and S participate in forming the core helical regions. For example, the P sequence pairs with Q contributing to the P4 helix, and the R sequence pairs with S contributing to the P7 helix. The active core of the Group I ribozyme contains a scaffolding domain P4/P6 (P4, P5 and P6) and a catalytic domain P3/P9 (P3, P7, P8 and P9). The P3-P7-P9 helix contains the GTP binding pocket. Base-pairing interactions between the 5′-end of the intron and flanking exon sequences define the location of the 5′ and 3′ splice sites. The 5′ splice site is determined by pairing of Internal Guide Sequence (IGS), a short intronic sequence near the 5′-end, with sequences of the upstream exon (5′ exon) to form P1. The 3′ splice site is determined by pairing of a short sequence of the downstream exon (3′ exon) with a portion of the IGS and mediating interactions between P9 and the P3/P8 helices that form the catalytic core.

The conserved P, Q, R, and S sequences, the IGS, and the P1 and P9 regions of a Group I intron can be determined using methods in the art in view of the present disclosure, for example, with reference to the following documents: Burke, J. M., et al., (1987) Structural conventions for group I introns; Stahley, R. M., et al (2006) RNA splicing: group I intron crystal structures reveal the basis of splice site selection and metal ion catalysis; and/or Woodson, A. S., (2005) Structure and assembly of group I introns.

Splicing of the Group I intron RNA is done by a two-step transesterification reaction. The first reaction is initiated by the 3′—OH group of an exogenous GTP that docks into the G-binding pocket located in the P7 region and the 3′—OH group attacks the 5′ splice site. In the second reaction, the 3′-OH of the released 5′ exon attacks the phosphodiester bond between the intronic terminal G and the 3′ exon, resulting in the liberation of the intron and the ligation of the exons. See, e.g., Hausner et al., Mobile DNA, volume 5, Article number: 8 (2014). Group I introns are also described in detail in, for example, Nielsen H, Johansen S D (2009). “Group I introns: Moving in new directions”. RNA Biol. 6 (4): 375-83; Cate J H, Gooding A R, Podell E, et al. (September 1996). “Crystal structure of a group I ribozyme domain: principles of RNA packing”. Science. 273 (5282): 1678-85; Cech T R (1990). “Self-splicing of group I introns”. Annu. Rev. Biochem. 59: 543-68; Woodson SA (June 2005). “Structure and assembly of group I introns”. Curr. Opin. Struct. Biol. 15 (3): 324-30.

In one general aspect, the application relates to a pair of RNA molecules for making a circular RNA, comprising:
(1) a first RNA molecule comprising the following elements operably connected and arranged in the order from the 5′-end to the 3′-end of the molecule:
   i) a 3′ intron sequence of a Group I intron,
   ii) a 3′ splice site of the Group I intron,
   iii) optionally, a 3′ exon sequence of the Group I intron,
   iv) an RNA sequence of interest,
   v) optionally, a 5′ exon sequence of the Group I intron,
   vi) a 5′ splice site of the Group I intron, and
   vii) a 5′ intron sequence of the Group I intron, and
(2) a second RNA molecule comprising an intermediate intron sequence of the Group I intron,
wherein when the first RNA molecule is contacted with the second RNA molecule, a scaffolding domain and a catalytic domain of the Group I intron are formed to thereby allow the production of the circular RNA comprising the 3′ exon sequence, the RNA sequence of interest and the 5′ exon sequence.

As used herein, a “3′ intron sequence of a Group I intron” refers to an RNA sequence derived from a sequence that is proximal to the 3′-end of the Group I intron. In certain embodiments, the “3′ intron sequence of a Group I intron” comprises the R sequence and the S sequence of the Group I intron. In certain embodiments, the “3′ intron sequence of a Group I intron” is at least at least 50%, at least 60%, at least 70%, at least 80%, at least 90%, at least 91%, at least 92%, at least 93%, at least 94%, at least 95%, at least 96%, at least 97%, at least 98%, at least 99% or 100% identical to a native sequence that is proximal to the 3′-end of the Group I intron. For example, the “3 intron sequence of a Group I intron” can have 1, 2, 3, 4, 5, 6, 7, 8, 9, or 10 or more nucleotide substitutions, deletions or additions compared to a corresponding native sequence.

As used herein, a “5′ intron sequence of a Group I intron” refers to an RNA sequence derived from a Group I intron that is proximal to the 5′-end of the Group I intron. In certain embodiments, the “5′ intron sequence of a Group I intron” comprises the internal guide sequence (IGS) and the P sequence of the Group I intron. In certain embodiments, the “5′ intron sequence of a Group I intron” is at least at least 50%, at least 60%, at least 70%, at least 80%, at least 90%, at least 91%, at least 92%, at least 93%, at least 94%, at least 95%, at least 96%, at least 97%, at least 98%, at least 99% or 100% identical to a native sequence that is proximal to the 5′-end of the Group I intron. For example, the “5′ intron sequence of a Group I intron” can have 1, 2, 3, 4, 5, 6, 7, 8, 9, or 10 or more nucleotide substitutions, deletions or additions compared to a corresponding native sequence.

As used herein, an “intermediate intron sequence of the Group I intron” refers to an RNA sequence derived from a Group I intron that is between a native 5′ intron sequence and a native 3′ intron sequence of a Group I intron. In certain embodiments, the “intermediate intron sequence of the Group I intron” comprises the Q sequence of the Group I self-splicing. In certain embodiments, the “intermediate intron sequence of the Group I intron” is at least at least 50%, at least 60%, at least 70%, at least 80%, at least 90%, at least 91%, at least 92%, at least 93%, at least 94%, at least 95%, at least 96%, at least 97%, at least 98%, at least 99% or 100% identical to a native sequence that is between a native 5′ intron sequence and a native 3′ intron sequence of a Group I intron. For example, the “intermediate intron sequence of the Group I intron” can have 1, 2, 3, 4, 5, 6, 7, 8, 9, or 10 or more nucleotide substitutions, deletions or additions compared to a corresponding native sequence.

A “splice site” as used here refer to a dinucleotide sequence where the covalent bond between its two nucleotides is cleaved during a splicing reaction. A “5′-splice site” refers to a splice site at the 5′ end of a Group I intron, while a “3-splice site” refers to a splice site at the 3′ end of a Group I intron. Introns are removed from primary transcripts by cleavage at 5′ and 3′ splice sites. In some embodiments, the 5′-splice site contains the sequence of UA. In some embodiments, the 5′-splice site contains the sequence of UU. In some embodiments, the 3′-splice site contains the sequence of UG. In some embodiments, the RNA sequence that is removed begins with the dinucleotide UA at its 5′ end and ends with UG at its 3′ end. In some embodiments, the RNA sequence that is removed begins with the dinucleotide UU at its 5′ end and ends with UG at its 3′ end.

As used herein, “exon” or “exon sequence” refers to a sequence derived from a native exon sequence that flanks a Group I intron and is capable of being recognized and/or spliced by the Group I intron. In some embodiments, a 3′ exon sequence (i.e., the sequence of an exon proximal to the 3′ splicing site or a fragment thereof) and a 5′ exon sequence (i.e., the sequence of an exon proximal to the 5′ splicing site or a fragment thereof) are both required for making a circular RNA using a method according to the application. In other embodiments, a 3′ exon sequence and/or a 5′ exon sequence are not required for making a circular RNA using a method according to the application. See, e.g., Rausch et al., Nucleic Acid Research, 2021, 49(6), e35, the content of which is incorporated herein by reference in its entirety. In preferred embodiments, the 3′ exon sequence is derived from a native 3′ exon sequence of the Group I intron, i.e., an exon sequence flanking (downstream of) the 3′ splicing site, or a contiguous fragment thereof starting from the 5′ terminal nucleotide of the native 3′ exon sequence. In some embodiments, the 3′ exon sequence includes the entire native sequence of an exon proximal to the 3′ splicing site of the Group I intron, or has at least 50%, at least 60%, at least 70%, at least 80%, at least 90%, at least 91%, at least 92%, at least 93%, at least 94%, at least 95%, at least 96%, at least 97%, at least 98%, or at least 99% sequence identity to the entire native sequence or a continuous fragment thereof starting from the 5′ terminal nucleotide of the native 3′ exon sequence. In some embodiment, the 3′ exon sequence has 1, 2, 3, 4, 5, 6, 7, 8, 9, or 10 or more nucleotide substitutions, deletions or additions compared to the entire native 3′ exon sequence, or a continuous fragment thereof starting from the 5′ terminal nucleotide of the native 3′ exon sequence.

In preferred embodiments, the 5′ exon sequence is derived from a native 5′ exon sequence of the Group I intron, i.e., an exon sequence flanking (upstream of) the 5′ splicing site, or a contiguous fragment thereof starting from the 5′ terminal nucleotide of the native 5′ exon. In some embodiments, the 5′ exon sequence includes the entire native sequence of an exon proximal to the 5′ splicing site of the Group I intron, or has at least 50%, at least 60%, at least 70%, at least 80%, at least 90%, at least 91%, at least 92%, at least 93%, at least 94%, at least 95%, at least 96%, at least 97%, at least 98%, or at least 99% sequence identity to the entire native sequence of an exon proximal to the 5′ splicing site of the Group I intron, or a continuous fragment thereof starting from the 5′ terminal nucleotide of the native 5′ exon. In some embodiment, the 5′ exon sequence has 1, 2, 3, 4, 5, 6, 7, 8, 9, or 10 or more nucleotide substitutions, deletions or additions compared to the entire native 5′ exon of the self-splicing intron, or a continuous fragment thereof starting from the 3′ terminal nucleotide of the native 5′ exon.

In certain embodiments, the 5′ exon sequence comprises or consists of the nucleotide sequence of SEQ ID NO: 59 (ACGGACUU). In some embodiments, the 3′ exon sequence comprises or consists of the nucleotide sequence of SEQ ID NO: 60 (AAAAUCCGU). In one embodiment, the first RNA molecules comprise a 3′ exon sequence having of the nucleotide sequence at least 80%, 85%, 90%, 95% or 100% identical to SEQ ID NO: 60 or SEQ ID NO: 3. In another embodiment, the first RNA molecules comprise a 5′ exon sequence having of the nucleotide sequence at least 80%, 85%, 90%, 95% or 100% identical to SEQ ID NO: 59 or SEQ ID NO: 4.

In certain embodiments, when the first RNA molecule is contacted with the second RNA molecule, the first, second and third intron fragments interact with each other to form the secondary structure of paired (P) elements necessary for the formation of the scaffolding domain and the catalytic domain. The P element is also described as “P region” or “P stem-loop region” in the application.

The first and second RNA molecules can be made by any suitable methods in view of the present disclosure. In certain embodiments, the first and second RNA molecules can be obtained by permuting a Group I intron. For example, the first RNA molecule can comprise, from the 5′-end to the 3′-end of the molecule: (1) Fragment 3 comprising the 3′ intron sequence and the 3′ splice site operably connected and arranged in the order from the 5′-end to the 3′-end of Fragment 3; (2) the 3′ exon sequence; (3) the RNA sequence of interest; (4) the 5′ exon sequence; and (5) Fragment 1 comprising the 5′ splice site and the second intron fragment operably connected and arranged in the order from the 5′-end to the 3′-end of Fragment 1. The second RNA molecule can comprise Fragment 2 having the intermediate intron sequence. One or more of Fragments 1-3 can be derived from portions of the Group I intron obtained by dividing the intron with two interrupted sites.

An “interrupted site” or “splitting site” as used herein, refers to a site in a Group I intron where a cut is made to obtain RNA fragments of the intron. This cut generates RNA fragments of a Group I intron that can be permutated or arranged to assist the circularization an RNA sequence of interest. According to embodiments of the application, two interrupted sites are selected to allow generating three separate portions of a Group I intron (e.g., Fragment 1, Fragment 2, and Fragment 3) which together can maintain ribozyme activity necessary for the self-splicing, even after permutation. The three separate fragments maintaining the overall conformation of the Group I intron can maintain ribozyme activity necessary for the self-splicing.

In certain embodiments, one interrupted site is located within a loop of the P6 stem-loop region of the Group I intron, and the other interrupted site is located within a loop of the P2, P5, P8, or P9 stem-loop region of the Group I intron. In one embodiment, the three portions of the Group I intron can be obtained by dividing the intron with a first interrupted site located within a loop of the P2 stem-loop region, and a second interrupted site located within a loop of the P6 stem-loop. In another embodiment, the three portions of the Group I intron are obtained by dividing the intron with a first interrupted site located within a loop of the P5 stem-loop region, and a second interrupted site located within a loop of the P6 stem-loop. In yet another embodiment, the three portions of the Group I intron are obtained by dividing the intron with a first interrupted site located within a loop of the P6 stem-loop region, and a second interrupted site located within a loop of the P8 stem-loop. In a further embodiment, the three portions of the Group I intron are obtained by dividing the intron with a first interrupted site located within a loop of the P6 stem-loop region, and a second interrupted site located within a loop of the P9 stem-loop.

In certain embodiments, Fragment 1 comprises a nucleotide sequence that is at least 80% identical, such as at least 80%, 85%, 90%, 91%, 92%, 93%, 94%, 95%, 96%, 97%, 98%, 99% or 100% identical, to the nucleotide sequence from the 5′ end of the Group I intron to the left side of the first interrupted site. Fragment 2 comprises a nucleotide sequence that is at least 80% identical, such as at least 80%, 85%, 90%, 91%, 92%, 93%, 94%, 95%, 96%, 97%, 98%, 99% or 100% identical, to the nucleotide sequence from the right side of the first interrupted site to the left side of the second interrupted site. Fragment 3 comprises a nucleotide sequence that is at least 80% identical, such as at least 80%, 85%, 90%, 91%, 92%, 93%, 94%, 95%, 96%, 97%, 98%, 99% or 100% identical, to the nucleotide sequence from the right side of the second interrupted site to the 3′ end of the Group I intron. The first and second interrupted sites are numbered according to their occurrence as one traces the RNA chain from 5′ to 3′.

According to embodiments of the application, Fragment 1 can comprise a nucleotide sequence that is at least 80% identical, such as at least 80%, 85%, 90%, 91%, 92%, 93%, 94%, 95%, 96%, 97%, 98%, 99% or 100% identical, to the nucleotide sequence of SEQ ID NO: 11, 14, 17 or 20. Fragment 2 can comprise a nucleotide sequence that is at least 80% identical, such as at least 80%, 85%, 90%, 91%, 92%, 93%, 94%, 95%, 96%, 97%, 98%, 99% or 100% identical, to the nucleotide sequence of SEQ ID NO: 12, 15, 18, 21, 23, 25, 27 or 29. Fragment 3 can comprise a nucleotide sequence that is at least 80% identical, such as at least 80%, 85%, 90%, 91%, 92%, 93%, 94%, 95%, 96%, 97%, 98%, 99% or 100% identical, to the nucleotide sequence of SEQ ID NO: 13, 16, 19, 22, 24, 26, 28 or 30.

Preferably, a pair of RNA molecules of the application comprise Fragment 1, Fragment 2 and Fragment 3 having the nucleotide sequence of:
a. SEQ ID NO: 11, SEQ ID NO:12, and SEQ ID NO: 13, respectively;
b. SEQ ID NO: 14, SEQ ID NO:15, and SEQ ID NO: 16, respectively;
c. SEQ ID NO: 17, SEQ ID NO: 18, and SEQ ID NO: 19, respectively;
d. SEQ ID NO: 20, SEQ ID NO: 21, and SEQ ID NO: 22, respectively;
e. SEQ ID NO: 14, SEQ ID NO: 23, and SEQ ID NO: 24, respectively;
f. SEQ ID NO: 14, SEQ ID NO: 25, and SEQ ID NO: 26, respectively;
g. SEQ ID NO: 14, SEQ ID NO: 27, and SEQ ID NO: 28, respectively; or
h. SEQ ID NO: 14, SEQ ID NO: 29, and SEQ ID NO: 30, respectively

In preferred embodiments, the Group I intron is from Cyanobacterium Anabaena, a T4 phage, or Azoarcus sp. BH72. Preferably, the Group I intron is from a Cyanobacterium Anabaena sp. pre-tRNA-Leu gene, a td gene of T4 phage, or a pre-tRNA-Ile gene of Azoarcus sp. BH72.

In certain embodiments, the three RNA fragments derived from a Group I intron can be used for the preparation of a pair of RNA molecules of the application that retain the self-splicing activity for making a circular RNA. For example, a pair of RNA molecules according to an embodiment of the application can comprise: 1) a first RNA molecule comprising the following elements operably connected and arranged in the order from the 5′-end to the 3′-end of the molecule: i) a Fragment 3 according to the application that includes a 3′-intron sequence, a 3′-splice site of a Group 1 intron and a 3′ exon sequence of the Group 1 intron; ii) a ribonucleotide sequence of interest; iii) a Fragment 1 according to the application that includes a 5′ exon sequence, a 5′-splice site and a 5′-intron sequence of the Group 1 intron; and 2) a second RNA molecule comprising a Fragment 2 according to the application that includes an intermediate intron sequence of the Group I intron. The first RNA molecule is a permuted RNA molecule that allows the generation of a circular RNA comprising the nucleotide sequence of interest through the self-splicing of the permuted RNA molecule with the help of Fragment 2.

In certain embodiment, the nucleotide sequence of interest encodes a protein. Because circRNAs are covalently linked head to tail and lack a 5′ terminus, they lack a 7-methylguanylate (m7G) cap that is involved in the mRNA translation initiation. circRNAs must rely on cap-independent mechanisms, such as IRESs to initiate translation.

In preferred embodiments, the nucleotide sequence of interest comprises at least one protein-coding sequence and a translation initiation element such as an internal ribosome entry site (IRES) operably linked thereto. Here, “operably linked” refers to that the translation initiation element such as IRES can mediate translation of the encoded protein.

As used herein, the IRES sequence can be selected from, but is not limited to, the IRES sequences of the following organisms: Taura syndrome virus, blood-sucking bug virus, Tyler's encephalomyelitis virus, simian virus 40, red fire ant virus 1, cereal constriction virus, reticulovirus Endothelial hyperplasia virus, Forman poliovirus 1, soybean inchworm virus, Kashmir bee virus, human rhinovirus 2, glass leafhopper virus-1, human immunodeficiency virus type 1, glass leafhopper virus-1, lice P virus, Hepatitis C virus, Hepatitis A virus, Hepatitis GB virus, Foot and mouth disease virus, Human enterovirus 71, Equine rhinovirus, Tea inchworm-like virus, Encephalomyocarditis virus (EMCV), Drosophila C virus, Cruciferous tobacco Virus, cricket paralysis virus, bovine viral diarrhea virus 1, black queen cell virus, aphid lethal paralysis virus, avian encephalomyelitis virus, acute bee paralysis virus, hibiscus yellow ring spot virus, swine fever virus, Aichi virus, kobuvirus, parechovirus, apthovirus, enterovirus, bopivirus, diresapivirus, Aalivirus, Ailurivirus, Ampivirus, Anativirus, Aphthovirus, Avihepatatovirus, Avisivirus, Boosepivirus, Caridiovirus, cosavirus, Crahelivirus, Crohivirus, Dicipivirus, Diresapivirus, entervirus, Eribovirus, Felipivirus, Fipivirus, Galivirus, Grusopivirus, Harkavirus, Hemipivirus, Hepatovirus, Hummivirus, Kunsagivirus, Limnipivirus, Livupivirus, Ludopivirus, Malagasivirus, Megrivirus, Mischivirus, Mosavirus, Mupivirus, Orivirus, parabovirus, parebovirus, pasivirus, passerivirus, pemapivirus, potamipivirus, rabovirus, rafivirus, rohelivirus, rosavirus, sakovuvirus, salivirus, sapelovirus, senecavirus, shanbavirus, sicinvirus, symapivirus, symapivirus, teschovirus, torchivirus, tottorivirus, tremovirus, tropivirus, human FGF2, human SFTPA1, Human AML1/RUNX1, Drosophila Antennae, Human AQP4, Human AT1R, Human BAG-1, Human BCL2, Human BiP, Human c-IAPl, Human c-myc, Human eIF4G, Mouse NDST4L, Human LEF1, Mouse HIFla, Human n.myc, mouse Gtx, human p27kipl, human PDGF2/c-sis, human p53, human Pim-1, mouse Rbm3, Drosophila reaper, canine Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, simian picomavirus, turnip crepe disease virus, eIF4G aptamer, Coxsackie Virus B3 (CVB3) or Coxsackie virus A (CVA1/2). Wild-type IRES sequences can also be modified and used in the present application. Preferably, the IRES is CVB3, BRAV-1L, PV1L, CAV2L, BRAV-1, PV1, or CAV2.

In certain embodiments, the nucleotide sequence of interest can include additional sequences to enhance the translation. For example, the nucleotide sequence of interest can further include a 5′-UTR and a 3′-UTR flanking the IRES and protein-coding sequence. The nucleotide sequence of interest can further include a spacer between the IRES and the 5′-end of the protein-coding sequence. The nucleotide sequence of interest can also include a spacer between the 3′-end of the coding sequence and the 3′-UTR.

The nucleotide sequence of interest can include a coding sequence for any protein of interest. The encoded protein can be any protein for therapeutic or diagnostic use. For example, the encoded protein can be a secreted protein, an intracellular protein, or a nuclear protein in eukaryotic cells. In some embodiments, the encoded protein is a human protein, an antigen, an antibody, or a gene editing enzyme (such as a CRISPR nuclease). In other embodiments, the encoded protein is a chimeric antigen receptor, an immunomodulatory protein, a transcription factor, and the like.

In other embodiments, the nucleotide sequence of interest is a non-protein coding sequence. For example, the non-protein-coding sequence can be antisense RNA, aptamer, guide RNA, or non-protein-coding RNA existing in any organism, and the like. The non-protein coding sequence may or may not contain a specific secondary structure.

In some embodiments, the nucleotide sequence of interest is at least 10, 20, 40, 60, 80, 100, 200, 300, 400, 500, 600, 700, 800, 900, 1000, 1500, 2000, 2500, 3000, 3500, 4000, 4500, 10000, 20000 nucleotides in length. In some embodiments, the nucleotide sequence of interest is about 10- about 20000 nucleotides in length.

An RNA molecule useful for an invention according to embodiments of the application can be (e.g., chemically) unmodified, partially modified or fully modified. In some embodiments, a first RNA molecule and/or a second RNA molecule of the application comprise at least one modified nucleotide. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides in the first and/or second RNA molecule(s) are modified nucleotides. It is not necessary for all positions in a given RNA molecule to be uniformly modified. More than one modification can be incorporated in the RNA molecule. Modification at one nucleotide is independent of modification at another nucleotide. The RNA molecules can be synthesized and/or modified using methods known in the art in view of the present disclosure.

Modified nucleotides can be a modified nucleoside, a modified phosphate, and/or a modified internucleotide linkage. For example, modified nucleotides include, but are not limited to, 5-methylcytosine (m5C), N6-methyladenosine (m6A), pseudouridine (ψ), Nlmethylpseudouridine ( [symbol] ), 5-methoxyuridine (5moU), 2′-deoxy-2′-fluoro-ribose (2′-F), a 2′ O-methyl ribose (2′ O-Me) or the acyclic sugar of an UNA nucleotide, a phosphorothioate linkage, or a thiophosphoramidate linkage. In some embodiments, at least one of the nucleotide modifications is a cytidine modification, a uridine modification, or an adenosine modification. In some embodiments, at least one of the modified nucleosides is selected from the group consisting of 5-methylcytosine (m5C), N6-methyladenosine (m6A), pseudouridine (ψ), Nlmethylpseudouridine ( [symbol] ) and 5-methoxyuridine (5moU).

In some preferred embodiments, the RNA molecule comprises less than 100%, less than 90%, less than 80%, less than 70%, less than 60%, less than 50%, less than 40%, less than 30%, less than 20%, less than 15%, less than 10%, less than 5%, less than 1% of a specific nucleotide modification. As used herein, the percentage of a particular nucleotide modification refers to the ratio of nucleotides in the sequence that have undergone that particular modification to nucleotides that can undergo that particular modification.

In some preferred embodiments, the RNA molecule is unmodified. In some embodiments, the RNA molecule does not contain nucleotide chemical modification.

In some embodiments, a first RNA molecule of the application further comprises a 3′-end binding motif, and a second RNA molecule of the application further comprises a 5′-end binding motif, wherein the 3′-end binding motif and the 5′-end binding motif are capable of complementary pairing to form a double-stranded region. Preferably, the 3′-end binding motif and the 5′-end binding motif are perfectly complimentary to each other.

In some embodiments, the binding motifs can be, for example, about 5-50 nucleotides in length, for example, about 5-50, about 10-50, about 20-50, about 30-50, about 40-50, about 5-40, about 5-30, about 5-20, or about 10-20 nucleotides in length. In some embodiments, the homology arm is 20 nucleotides in length. In some embodiments, the homology arm is 40 nucleotides in length. In certain embodiments, the homology arm is at least 5, 6, 7, 8, 9, 10, 11, 12, 13, 14, 15, 16, 17, 18, 19, or 20 nucleotides in length. In certain embodiments, the homology arm is no more than 50, 45, 40, 35, 30, 25, or 20 nucleotides in length. In certain embodiments, the homology arm is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length.

In some embodiments, the double-stranded region formed by the 3′-end binding motif and the 5′-end binding motif is about 5-50, about 5-40, about 5-30, about 5-20, or about 10-20 nucleotides in length, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. Preferably, the double-stranded region is 8, 9, 10, 11, 12, 13, 14, 15, 16, 17, 18, 19, 20, 21, 22, 23, 24, 25 nucleotides in length.

Another general aspect of the application relates to a nucleic acid that encodes the first and/or second RNA molecules of the application. The first and second RNA molecules of the application can be encoded by the one DNA molecule or two separate DNA molecules.

The application also relates to a nucleic acid vector comprising or encoding the first and/or second RNA molecules. As used herein, a “vector” or “nucleic acid vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). A vector can be a DNA vector or an RNA vector. One of ordinary skill in the art can construct a vector of the application through standard recombinant techniques in view of the present disclosure.

A vector of the application can be an expression vector. As used herein, the term “expression vector” refers to any type of genetic construct comprising a nucleic acid coding for an RNA capable of being transcribed. Expression vectors include, but are not limited to, vectors for recombinant expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

A vector of the application can also be a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, Modified Vaccinia virus Ankara (MVA) vectors, lentiviral vectors, etc.

In preferred embodiments, the nucleic acid vector further comprises an RNA polymerase promoter sequence operably linked to the coding sequence of the RNA molecules. The operably linked promoter allows in vivo and/or in vitro transcription of the coding sequence. The promoter can be, for example, a T7 RNA polymerase promoter, a T6 viral RNA polymerase promoter, a SP6 viral RNA polymerase promoter, a T3 viral RNA polymerase promoter or a T4 viral RNA polymerase promoter.

In certain embodiments, the nucleic acid vector” can be a DNA derived from a virus, plasmid or cell of a higher organism into which a foreign DNA fragment can be or has been inserted for cloning and/or expression purposes. In certain embodiments, the vector can be stably maintained in the organism. The vector can contain, for example, an origin of replication, a selectable marker or a reporter gene, such as antibiotic resistance or GFP, and/or a multiple cloning site (MCS). The term includes linear DNA fragments (e.g., PCR products, linear plasmid fragments), plasmid vectors, viral vectors, cosmids, bacterial artificial chromosomes (BACs), yeast artificial chromosomes (YACs), and the like.

In another general aspect, the present application provides a method for preparing a circular RNA, wherein the method comprises:
i) providing or obtaining a pair of RNA molecules according to embodiments of the application;
(ii) adding a buffer solution to the pair of RNA molecules to obtain a reaction mixture to allow the formation of the scaffolding domain and the catalytic domain of the Group I intron, and
(iii) adding GTP and a divalent metal cation to the reaction mixture at a temperature to allow the formation of the circular RNA,
optionally, the method further comprises:
(iv) harvesting the circular RNA formed in step iii).

In certain embodiments, the first RNA molecule and the second RNA molecule in the pair of RNA molecules are provided at a molar ratio of 1:1; 1:2; 1:3; 1:4; or 1:5, preferably at the molar ratio of 1:1.

Any suitable buffer can be used for the method of the application in view of the present disclosure. For example, the buffer can comprise 10-200 mM Tris-HCl, such as 50 mM Tris-HCl, at pH 6-8.5, such as pH 6, 6.5, 7, 7.5, 8 or 8.5. In a preferred embodiment, the reaction mixture comprises 50 mM Tris-HCl, pH 7.5.

In certain embodiments, the GTP is added to the reaction mixture at a concentration of 100 nM to 2 mM.

In certain embodiments, the divalent metal cation is added to the reaction mixture at a concentration of least about 5 mM, e.g., about 5 mM to about 550 mM, e.g., at least about 5 mM, about 10 mM, about 15 mM, at least about 20 mM, at least about 30 mM, at least about 40 mM, at least about 50 mM, at least about 60 mM, at least about 70 mM, at least about 80 mM, at least about 90 mM, at least about 100 mM, at least about 125 mM, at least about 150 mM, at least about 175 mM, at least about 200 mM, at least about 250 mM, at least about 300 mM, at least about 350 mM, at least about 400 mM, at least about 450 mM, at least about 500 mM, at least about 550 mM or higher. Any suitable divalent metal cation ions can be used, such as Mg2+.

In some embodiments, the pair of RNA molecules are encoded by one or more DNA molecules that are non-naturally occurring and can be produced using recombinant technology (methods described in detail below, e.g., derived in vitro using a DNA plasmid) or chemical synthesis. In some other embodiments, the pair of RNA molecules are chemically synthesized. It is within the scope of the application that a DNA molecule used to produce a circular RNA can comprise a DNA sequence of a naturally occurring original nucleic acid sequence, a modified version thereof, or a DNA sequence encoding a synthetic polypeptide not normally found in nature (e.g., chimeric molecules or fusion proteins). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

The inventors have surprisingly found that a method of preparing a circular RNA provided in the application results in a higher self-splicing efficiency with lower side products, such as dimer or concatemer, as compared to the conventional PIE method. The self-splicing efficiency depends on proper folding and assembly of the Group I intron RNA. Circularization efficiency can be measured by the ratio of outcome circular RNA in a given time period. The dimer or concatemer is a side product of an oligomer containing two or more monomers of precursor RNA joined by intermolecular non-covalent bonds that can be either strong or weak. The self-splicing efficiency, circularization efficiency and percentage of dimer or concatemer can be determined by methods known in the art, such as those described in Examples herein.

In some embodiments, compared with a conventional PIE method, a method of the application results in a self-splicing efficiency that is increased by at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500% or more.

In some embodiments, a method of the application results in no or substantially no dimer or concatemer side products.

Another general aspect of the application relates to a circular RNA produced by a method of the application, its pharmaceutical compositions and uses thereof. The circular RNA comprises a nucleotide sequence of interest that encodes a protein or non-protein.

In some embodiments, the present application provides a pharmaceutical composition comprising a nucleic acid vector of the present application, a combination of three RNA fragments of a Group I intron, a pair of RNA molecules and/or a circular RNA of the application, and a pharmaceutically acceptable carrier.

The specific use of the composition can depend on the nucleotide sequence of interest. In some embodiment, the pharmaceutical composition is for use in treating a disease in a subject. The specific disease to be treated can depend on the specific nucleotide sequence of interest.

Pharmaceutically acceptable carriers can include, but are not limited to, buffers, excipients, stabilizers or preservatives. Examples of pharmaceutically acceptable carriers are physiologically compatible solvents, dispersion media, coatings, antibacterial and antifungal agents, isotonic and absorption delaying agents, and the like, such as salts, buffers, carbohydrates, antioxidants, aqueous or non-aqueous carriers, preservatives, wetting agents, surfactants or emulsifying agents or combinations thereof. The amount of a pharmaceutically acceptable carrier in a pharmaceutical composition can be determined experimentally based on the activity of the carrier and the desired properties of the formulation, such as stability and/or minimal oxidation.

In further embodiments, the pharmaceutical compositions may optionally comprise one or more additional active substances, e.g., therapeutically and/or prophylactically active substances. Pharmaceutical compositions of the present application may be sterile and/or pyrogen-free. General considerations in the formulation and/or manufacture of pharmaceutical agents may be found, for example, in Remington. The Science and Practice of Pharmacy 21st ed., Lippincott Williams & Wilkins, 2005 (incorporated herein by reference).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

A circular RNA can be delivered to a subject in need thereof via any suitable delivery systems. Delivery systems can include time-released, delayed release, and sustained release delivery systems such that the delivery of the compositions occurs prior to, and with sufficient time to cause, sensitization of the site to be treated. The composition can be used in conjunction with other therapeutic agents or therapies. Such systems can avoid repeated administrations of the composition, thereby increasing convenience to the subject and the physician, and may be particularly suitable for certain composition embodiments of the application.

Release delivery systems include polymer base systems such as poly(lactide-glycolide), copolyoxalates, polyesteramides, polyorthoesters, polycaprolactones, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; sylastic systems; peptide based systems; hydrogel release systems; wax coatings; compressed tablets using conventional binders and excipients; partially fused implants; and the like. In some embodiments, lipid nanoparticles or polymers are used as delivery vehicles for therapeutic circRNAs described herein, including delivery of RNA to tissues.

Lipid nanoparticles have shown significant potential for use as delivery vehicles for therapeutic RNAs, including the delivery of mRNA to tissues (Oberli, M. A. et al. Lipid Nanoparticle Assisted mRNA Delivery for Potent Cancer Immunotherapy. Nano Lett. 17, 1326-1335 (2017); Yanez Arteta, M. et al. Successful reprogramming of cellular protein production through mRNA delivered by functionalized lipid nanoparticles. Proc. Natl. Acad. Sci. U.S.A 115, E3351-E3360 (2018); and Kaczmarek, J. C., Kowalski, P. S. & Anderson, D. G. Advances in the delivery of RNA therapeutics: from concept to clinical reality. Genome Med. 9, 60 (2017)). To assess the efficacy of lipid nanoparticles for circRNA delivery in vivo, purified circRNA was formulated into nanoparticles with the ionizable lipidoid cKK-E12 (Dong, Y. et al. Lipopeptide nanoparticles for potent and selective siRNA delivery in rodents and nonhuman primates. Proc. Natl. Acad. Sci. U.S.A 111, 3955-3960 (2014); and Kauffman, K. J. et al. Optimization of Lipid Nanoparticle Formulations for mRNA Delivery in Vivo with Fractional Factorial and Definitive Screening Designs. Nano Lett. 15, 7300-7306 (2015)).

The application further relates to a method for protein expression, comprising translating at least a region of the circular polyribonucleotide provided herein.

In some embodiments, the methods for protein expression comprises translation of at least 10%, at least 20%, at least 30%, at least 40%, at least 50%, at least 60%, at least 70%, at least 80%, at least 90%, or at least 95% of the total length of the circular RNA into polypeptides. In some embodiments, the methods for protein expression comprises translation of the circular RNA into polypeptides of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids. In some embodiments, the methods for protein expression comprises translation of the circular RNA into polypeptides of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, or about 1000 amino acids. In some embodiments, the methods comprise translation of the circular RNA into continuous polypeptides as provided herein, discrete polypeptides as provided herein, or both.

In some embodiments, the translation of the at least a region of the circular RNA takes place in vitro, such as rabbit reticulocyte lysate. In some embodiments, the translation of the at least a region of the circular RNA takes place in vivo, for instance, after transfection of a eukaryotic cell, or transformation of a prokaryotic cell such as a bacterium.

In some embodiments, the present disclosure provides methods of in vivo expression of one or more expression sequences in a subject, comprising: administering a circular RNA to a cell of the subject wherein the circular RNA comprises the one or more expression sequences; and expressing the one or more expression sequences from the circular RNA in the cell. In some embodiments, the circular RNA is configured such that expression of the one or more expression sequences in the cell at a later time point is equal to or higher than an earlier time point. In some embodiments, the circular RNA is configured such that expression of the one or more expression sequences in the cell over a time period of at least 7, 8, 9, 10, 12, 14, 16, 18, 20, 22, 23 or more days does not decrease by greater than about 40%. In some embodiments, the circular RNA is configured such that expression of the one or more expression sequences in the cell is maintained at a level that does not vary by more than about 40% for at least 7, 8, 9, 10, 12, 14, 16, 18, 20, 22, 23 or more days. In some embodiments, the administration of the circular RNA is conducted using any delivery method described herein. In some embodiments, the circular RNA is administered to the subject via intravenous injection. In some embodiments, the administration of the circular RNA includes, but is not limited to, prenatal administration, neonatal administration, postnatal administration, oral, by injection (e.g., intravenous, intraarterial, intraperotoneal, intradermal, subcutaneous and intramuscular), by ophthalmic administration and by intranasal administration.

In some embodiments, the methods for protein expression comprise modification, folding, or other post-translation modification of the translation product. In some embodiments, the methods for protein expression comprise post-translation modification in vivo, e.g., via cellular machinery.

All references and publications cited herein are hereby incorporated by reference.

The above-described embodiments can be combined to achieve the afore-mentioned functional characteristics. This is also illustrated by the below examples which set forth exemplary combinations and functional characteristics achieved.

### EMBODIMENTS

Provided below are exemplary numbered embodiments of the present invention.

Embodiment 1. A combination of three short RNA fragments derived from a Group I intron, wherein the three short RNA fragments are divided by two interrupted sites located within the Group I intron.

Embodiment 2. The combination of three short RNA fragments of Embodiment 1, wherein one interrupted site is located within P6 region, such as a loop of the P6 region, of the Group I Intron, the other interrupted site is located within P2, P5, P8, or P9 region, such as a loop of the P2, P5, P8, or P9 region of the Group I Intron.

Embodiment 3. The combination of three short RNA fragments of Embodiment 2, wherein one interrupted site is located within the P2 region, such as a loop of the P2 region, of the Group I Intron, the other interrupted site is located within the P6 region, such as a loop of the P6 region, of the Group I Intron.

Embodiment 4. The combination of three short RNA fragments of Embodiment 2, wherein one interrupted site is located within the P5 region, such as a loop of the P5 region, of the Group I Intron, the other interrupted site is located within the P6 region, such as a loop of the P6 region, of the Group I Intron.

Embodiment 5. The combination of three short RNA fragments of Embodiment 2, wherein one interrupted site is located within P6 region, such as a loop of the P6 region, of the Group I Intron, the other interrupted site is located within P8 region, such as a loop of the P8 region, of the Group I Intron.

Embodiment 6. The combination of three short RNA fragments of Embodiment 2, wherein one interrupted site is located within P6 region, such as a loop of the P6 region, of the Group I Intron, the other interrupted site is located within P9 region, such as a loop of the P9 region, of the Group I Intron.

Embodiment 7. The combination of three short RNA fragments of Embodiment 1 or 2, comprising:
i) Fragment 1 (F1) including a 5′-splice site, which is from the 5′ end of the Group I intron to the left side of a first interrupted site located within the Group I intron;
ii) Fragment 2 (F2), which is from the right side of the first interrupted site to the left side of a second interrupted site located within located within the Group I intron; and
iii) Fragment 3 (F3) including a 3′-splice site, which is from the right side of the second interrupted site to the 3′ end of the Group I intron.

Embodiment 8. The combination of three short RNA fragments of Embodiment 1 or 2, comprising:
i) Fragment 1 (F1) including a 5′-splice site, which is from the 5′ end of the Group I intron to the left side of a first interrupted site located within the P2 region, such as a loop of the P2 region;
ii) Fragment 2 (F2), which is from the right side of the first interrupted site to the left side of a second interrupted site located within the P6 region, such as a loop of the P6 region; and
iii) Fragment 3 (F3) including a 3′-splice site, which is from the right side of the second interrupted site to the 3′ end of the Group I intron.

Embodiment 9. The combination of three short RNA fragments of Embodiment 1 or 2, comprising:
i) Fragment 1 (F1) including a 5′-splice site, which is from the 5′ end of the Group I intron to the -left side of a first interrupted site located within the P5 region, such as a loop of the P5 region;
ii) Fragment 2 (F2), which is from the right side of the first interrupted site to the left side of a second interrupted site located within the P6 region, such as a loop of the P6 region; and
iii) Fragment 3 (F3) including a 3′-splice site, which is from to the right side of the second interrupted site to the 3′ end of the Group I intron.

Embodiment 10. The combination of three short RNA fragments of Embodiment 1 or 2, comprising:
i) Fragment 1 (F1) including a 5′-splice site, which is from the 5′ end of the Group I intron to the left side of a first interrupted site located within the P6 region, such as a loop of the P6 region;
ii) Fragment 2 (F2), which is from the right side of the first interrupted site to the left side of a second interrupted site located within P8 region, such as a loop of the P8 region; and
iii) Fragment 3 (F3) including a 3′-splice site, which is from the right side of the second interrupted site to the 3′ end of the Group I intron.

Embodiment 11. The combination of three short RNA fragments of Embodiment 1 or 2, comprising:
i) Fragment 1 (F1) including a 5′-splice site, which is from the 5′ end of the Group I intron to the left side of a first interrupted site located within P6 region, such as a loop of the P6 region;
ii) Fragment 2 (F2), which is from the right side of the first interrupted site to the left side of a second interrupted site located within P9 region, such as a loop of the P9 region; and
iii) Fragment 3 (F3) including a 3′-splice site, which is from the right side of the second interrupted site to the 3′ end of the Group I intron.

Embodiment 12. The combination of three short RNA fragments of any one of Embodiment 7-11, wherein the 5′-splice site is within a native 5′ exon region of the Group I intron, the 3′-splice site is within a native 3′ exon region of the Group I intron.

Embodiment 13. The combination of three short RNA fragments of any one of Embodiments 1-12, wherein the Group I intron is selected from IC3 subgroup of Group I introns.

Embodiment 14. The combination of three short RNA fragments of any one of Embodiments 1-13, wherein the Group I intron is from a Group I intron of cyanobacterium Anabaena, a T4 phage, or Azoarcus sp. BH72m; preferably, the Group I intron is from a Group I intron of cyanobacterium Anabaena.

Embodiment 15. The combination of three short RNA fragments of any one of Embodiments 1-14, wherein the Group I intron is from a Cyanobacterium Anabaena sp. pre-tRNA-Leu gene, a td gene of T4 phage, or a pre-tRNA-Ile gene of Azoarcus sp. BH72.

Embodiment 16. The combination of three short RNA fragments of Embodiment 1 or 2, wherein the Fragment 1 is a sequence having at least 75%, at least 80%, at least 85%, at least 90%, at least 95%, 100% sequence identity to SEQ ID NO: 11, the Fragment 2 is a sequence having at least 75%, at least 80%, at least 85%, at least 90%, at least 95%, 100% sequence identity to SEQ ID NO: 12, the Fragment 3 is a sequence having at least 75%, at least 80%, at least 85%, at least 90%, at least 95%, 100% sequence identity to SEQ ID NO:13.

Embodiment 17. The combination of three short RNA fragments of Embodiment 1 or 2, wherein the Fragment 1 is a sequence having at least 75%, at least 80%, at least 85%, at least 90%, at least 95%, 100% sequence identity to SEQ ID NO: 14, the Fragment 2 is a sequence having at least 75%, at least 80%, at least 85%, at least 90%, at least 95%, 100% sequence identity to SEQ ID NO: 15, the Fragment 3 is a sequence having at least 75%, at least 80%, at least 85%, at least 90%, at least 95%, 100% sequence identity to SEQ ID NO:16.

Embodiment 18. The combination of three short RNA fragments of Embodiment 1 or 2, wherein the Fragment 1 is a sequence having at least 75%, at least 80%, at least 85%, at least 90%, at least 95%, 100% sequence identity to SEQ ID NO: 17, the Fragment 2 is a sequence having at least 75%, at least 80%, at least 85%, at least 90%, at least 95%, 100% sequence identity to SEQ ID NO: 18, the Fragment 3 is a sequence having at least 75%, at least 80%, at least 85%, at least 90%, at least 95%, 100% sequence identity to SEQ ID NO:19.

Embodiment 19. The combination of three short RNA fragments of Embodiment 1 or 2, wherein the Fragment 1 is a sequence having at least 75%, at least 80%, at least 85%, at least 90%, at least 95%, 100% sequence identity to SEQ ID NO: 20, the Fragment 2 is a sequence having at least 75%, at least 80%, at least 85%, at least 90%, at least 95%, 100% sequence identity to SEQ ID NO: 21, the Fragment 3 is a sequence having at least 75%, at least 80%, at least 85%, at least 90%, at least 95%, 100% sequence identity to SEQ ID NO:22.

Embodiment 20. The combination of three short RNA fragments of Embodiment 1 or 2, wherein the Fragment 1 is a sequence having at least 75%, at least 80%, at least 85%, at least 90%, at least 95%, 100% sequence identity to SEQ ID NO: 14, the Fragment 2 is a sequence having at least 75%, at least 80%, at least 85%, at least 90%, at least 95%, 100% sequence identity to SEQ ID NO: 23, the Fragment 3 is a sequence having at least 75%, at least 80%, at least 85%, at least 90%, at least 95%, 100% sequence identity to SEQ ID NO:24.

Embodiment 21. The combination of three short RNA fragments of Embodiment 1 or 2, wherein the Fragment 1 is a sequence having at least 75%, at least 80%, at least 85%, at least 90%, at least 95%, 100% sequence identity to SEQ ID NO: 14, the Fragment 2 is a sequence having at least 75%, at least 80%, at least 85%, at least 90%, at least 95%, 100% sequence identity to SEQ ID NO: 25, the Fragment 3 is a sequence having at least 75%, at least 80%, at least 85%, at least 90%, at least 95%, 100% sequence identity to SEQ ID NO:26.

Embodiment 22. The combination of three short RNA fragments of Embodiment 1 or 2, wherein the Fragment 1 is a sequence having at least 75%, at least 80%, at least 85%, at least 90%, at least 95%, 100% sequence identity to SEQ ID NO: 14, the Fragment 2 is a sequence having at least 75%, at least 80%, at least 85%, at least 90%, at least 95%, 100% sequence identity to SEQ ID NO: 27, the Fragment 3 is a sequence having at least 75%, at least 80%, at least 85%, at least 90%, at least 95%, 100% sequence identity to SEQ ID NO:28.

Embodiment 23. The combination of three short RNA fragments of Embodiment 1 or 2, wherein the Fragment 1 is a sequence having at least 75%, at least 80%, at least 85%, at least 90%, at least 95%, 100% sequence identity to SEQ ID NO: 14, the Fragment 2 is a sequence having at least 75%, at least 80%, at least 85%, at least 90%, at least 95%, 100% sequence identity to SEQ ID NO: 29, the Fragment 3 is a sequence having at least 75%, at least 80%, at least 85%, at least 90%, at least 95%, 100% sequence identity to SEQ ID NO:30.

Embodiment 24. A pair of RNA molecules for making a circular RNA, comprising:
(1) a first RNA molecule comprising the following elements operably connected and arranged in the order from the 5′-end to the 3′-end of the molecule:
   i) a 3′ intron sequence of a Group I intron,
   ii) a 3′ splice site of the Group I intron,
   iii) optionally, a 3′ exon sequence of the Group I intron,
   iv) an RNA sequence of interest,
   v) optionally, a 5′ exon sequence of the Group I intron,
   vi) a 5′ splice site of the Group I intron, and
   vii) a 5′ intron sequence of the Group I intron, and
(2) a second RNA molecule comprising an intermediate intron sequence of the Group I intron,
wherein when the first RNA molecule is contacted with the second RNA molecule, a scaffolding domain and a catalytic domain of the Group I intron are formed to thereby allow the production of the circular RNA comprising the 3′ exon sequence, the RNA sequence of interest and the 5′ exon sequence.

Embodiment 24a. The pair of RNA molecules of Embodiment 24, wherein the 3′ intron sequence comprises the R sequence and the S sequence of the Group I intron, the 5′ intron sequence comprises the internal guide sequence (IGS) and the P sequence of the Group I intron, and the intermediate intron sequence comprises the Q sequence of the Group I intron.

Embodiment 24b. The pair of RNA molecules of Embodiment 24 or 24a, wherein the first RNA molecule comprises, from the 5′-end to the 3′-end of the molecule,
a) Fragment 3 comprising the 3′ intron sequence and the 3′ splice site operably connected and arranged in the order from the 5′-end to the 3′-end of Fragment 3;
b) optionally, the 3′ exon sequence;
c) the RNA sequence of interest;
d) optionally, the 5′ exon sequence;
e) Fragment 1 comprising the 5′ splice site and the 5′ intron sequence operably connected and arranged in the order from the 5′-end to the 3′-end of Fragment 1; and
the second RNA molecule comprises Fragment 2 having the intermediate intron sequence.

Embodiment 24c. The pair of RNA molecules of Embodiment 24b, wherein Fragments 1-3 comprise the nucleotide sequences or variants thereof of three portions of the Group I intron obtained by dividing the Group I intron with two interrupted sites, respectively.

Embodiment 24d. The pair of RNA molecules of Embodiment 24c, wherein the three portions of the Group I intron are obtained by dividing the intron with an interrupted site located within a loop of the P6 stem-loop region of the Group I intron, and another interrupted site located within a loop of the P2, P5, P8, or P9 stem-loop region of the Group I intron.

Embodiment 24e. A pair of RNA molecules that in combination retain the self-splicing activity to make a circular RNA, wherein the pair of RNA molecules comprises:
a) a permuted RNA molecule comprising the following elements operably connected to each other and, in some embodiments, arranged in the sequence from 5′ to 3′ direction in the following order:
   i) Fragment 3 including a 3′-splice site, which is from the right side of a second interrupted site to the 3′ end of the Group I intron;
   ii) optionally, a 3′ exon sequence;
   iii) a nucleotide sequence of interest;
   iv) optionally, a 5′ exon sequence; and
   v) Fragment 1 including a 5′-splice site, which is from the 5′ end of the Group I intron to the left side of a first interrupted site located within the Group I intron; and
b) Fragment 2, which is from the right side of the first interrupted site to the left side of the second interrupted site located within the Group I intron;
wherein Fragments 1-3 are obtained by dividing a Group I intron with two interrupted sites, wherein one interrupted site is located within the P6 region of the Group I Intron, and the other interrupted site is located within the P2, P5, P8, or P9 region of the Group I Intron;
wherein the permuted RNA molecule allows generation of a circular RNA comprising the nucleotide sequence of interest through the self-splicing of the permuted RNA molecule with the help of Fragment 2; and
wherein the first and second interrupted sites are numbered according to their occurrence as one traces the RNA chain from 5′ to 3′.

Embodiment 25. The pair of RNA molecules of any one of Embodiments 24-24e, wherein the 3′ exon sequence is derived from a native 3′ exon of the Group I intron.

Embodiment 25a. The pair of RNA molecules of any one of Embodiments 24-25, wherein the 3′ exon sequence is 100% identical to the native 3′ exon sequence of the Group I intron, or is at least 50%, at least 60%, at least 70%, at least 80%, at least 90%, at least 91%, at least 92%, at least 93%, at least 94%, at least 95%, at least 96%, at least 97%, at least 99%, or at least 99% identical to the full length sequence of the native 3′ exon sequence.

Embodiment 25b. The pair of RNA molecules of any one of Embodiments 24-25a, wherein the 3′ exon sequence has 1, 2, 3, 4, 5, 6, 7, 8, 9, or 10 or more nucleotide substitutions, deletions or additions compared to the native 3′ exon sequence.

Embodiment 25c. The pair of RNA molecules of any one of Embodiments 24-25b, wherein the 3′ exon sequence comprises SEQ ID NO: 60 (AAAAUCCGU).

Embodiment 25d. The pair of RNA molecules of any one of Embodiments 24-25b, wherein the 3′ exon sequence consists of SEQ ID NO: 60 (AAAAUCCGU).

Embodiment 25e. The pair of RNA molecules of any one of Embodiments 24-25b, wherein the 3′ exon sequence comprises a nucleotide sequence at least 80%, 85%, 90%, 95% or 100% identical to SEQ ID NO: 60.

Embodiment 25f. The pair of RNA molecules of any one of Embodiments 24-25b, wherein the 3′ exon sequence comprises SEQ ID NO: 3.

Embodiment 25g. The pair of RNA molecules of any one of Embodiments 24-25b, wherein the 3′ exon sequence consists of SEQ ID NO: 3.

Embodiment 25h. The pair of RNA molecules of any one of Embodiments 24-25b, wherein the 3′ exon sequence comprises a nucleotide sequence at least 80%, 85%, 90%, 95% or 100% identical to SEQ ID NO: 3.

Embodiment 25i. The pair of RNA molecules of any one of Embodiments 24-25h, wherein the first RNA molecule does not contain the 3′ exon sequence.

Embodiment 25j. The pair of RNA molecules of any one of Embodiments 24-25h, wherein the first RNA molecule contains the 3′ exon sequence.

Embodiment 26. The pair of RNA molecules of any one of Embodiments 24-25j, wherein the 5′ exon sequence is derived from a native 5′ exon of the Group I intron.

Embodiment 26a. The pair of RNA molecules of any one of Embodiments 24-26, wherein the 5′ exon sequence is 100% identical to the native 5′ exon sequence of the Group I intron, or is at least 50%, at least 60%, at least 70%, at least 80%, at least 90%, at least 91%, at least 92%, at least 93%, at least 94%, at least 95%, at least 96%, at least 97%, at least 98%, or at least 99% identical to the full length sequence of the native 5′ exon sequence.

Embodiment 26b. The pair of RNA molecules of any one of Embodiments 24-26a, wherein the 5′ exon sequence has 1, 2, 3, 4, 5, 6, 7, 8, 9, or 10 or more nucleotide substitutions, deletions or additions compared to the native 5′ exon sequence.

Embodiment 26c. The pair of RNA molecules of any one of Embodiments 24-26b, wherein the 5′ exon sequence comprises SEQ ID NO: 59 (ACGGACUU).

Embodiment 26d. The pair of RNA molecules of any one of Embodiments 24-26b, wherein the 5′ exon sequence consists of SEQ ID NO: 59 (ACGGACUU).

Embodiment 26e. The pair of RNA molecules of any one of Embodiments 24-26b, wherein the 5′ exon sequence comprises a nucleotide sequence at least 80%, 85%, 90%, 95% or 100% identical to SEQ ID NO: 59.

Embodiment 26f. The pair of RNA molecules of any one of Embodiments 24-26b, wherein the 5′ exon sequence comprises SEQ ID NO: 4.

Embodiment 26g. The pair of RNA molecules of any one of Embodiments 24-26b, wherein the 5′ exon sequence consists of SEQ ID NO: 4.

Embodiment 26h. The pair of RNA molecules of any one of Embodiments 24-26b, wherein the 5′ exon sequence comprises a nucleotide sequence at least 80%, 85%, 90%, 95% or 100% identical to SEQ ID NO: 4.

Embodiment 26i. The pair of RNA molecules of any one of Embodiments 24-26h, wherein the first RNA molecule does not contain the 5′ exon sequence.

Embodiment 26j. The pair of RNA molecules of Embodiment 26i, wherein the first RNA molecule also does not contain the 3′ exon sequence.

Embodiment 26k. The pair of RNA molecules of any one of Embodiments 24-26h, wherein the first RNA molecule contains the 5′ exon sequence.

Embodiment 26l. The pair of RNA molecules of Embodiments 26k, wherein the first RNA molecule also contains the 3′ exon sequence.

Embodiment 27. The pair of RNA molecules of any one of Embodiments 24-26l, wherein the first interrupted site is located within the P2 region, such as a loop of the P2 region, of the Group I Intron, the other interrupted site is located within the P6 region, such as a loop of the P6 region, of the Group I Intron.

Embodiment 28. The pair of RNA molecules of any one of Embodiments 24-26l, wherein the first interrupted site is located within the P5 region, such as a loop of the P5 region, of the Group I Intron, the second interrupted site is located within the P6 region, such as a loop of the P6 region, of the Group I Intron.

Embodiment 29. The pair of RNA molecules of any one of Embodiments 24-26l, wherein the first interrupted site is located within the P6 region, such as a loop of the P6 region, of the Group I Intron, the other interrupted site is located within the P8 region, such as a loop of the P8 region, of the Group I Intron.

Embodiment 30. The pair of RNA molecules of any one of Embodiments 24-26l, wherein the first interrupted site is located within the P6 region, such as a loop of the P6 region, of the Group I Intron, the other interrupted site is located within the P9, such as a loop of the P9 region, region of the Group I Intron.

Embodiment 31. The pair of RNA molecules of any one of Embodiments 24-30, wherein Fragments 1-3 are defined as any one of Embodiments 16-23.

Embodiment 32. The pair of RNA molecules of any one of Embodiments 24-31, wherein Fragment 1 comprises a nucleotide sequence that is at least 80% identical, such as at least 80%, 85%, 90%, 91%, 92%, 93%, 94%, 95%, 96%, 97%, 98%, 99% or 100% identical, to the nucleotide sequence from the 5′ end of the Group I intron to the left side of the first interrupted site.

Embodiment 32a. The pair of RNA molecules of any one of Embodiments 24-32, wherein Fragment 1 comprises a nucleotide sequence that is at least 80% identical, such as at least 80%, 85%, 90%, 91%, 92%, 93%, 94%, 95%, 96%, 97%, 98%, 99% or 100% identical, to the nucleotide sequence of SEQ ID NO: 11, 14, 17 or 20.

Embodiment 32b. The pair of RNA molecules of any one of Embodiments 24-32a, wherein Fragment 2 comprises a nucleotide sequence that is at least 80% identical, such as at least 80%, 85%, 90%, 91%, 92%, 93%, 94%, 95%, 96%, 97%, 98%, 99% or 100% identical, to the nucleotide sequence from the right side of the first interrupted site to the left side of the second interrupted site.

Embodiment 32c. The pair of RNA molecules of any one of Embodiments 24-32b, wherein Fragment 2 comprises a nucleotide sequence that is at least 80% identical, such as at least 80%, 85%, 90%, 91%, 92%, 93%, 94%, 95%, 96%, 97%, 98%, 99% or 100% identical, to the nucleotide sequence of SEQ ID NO: 12, 15, 18, 21, 23, 25, 27 or 29.

Embodiment 32d. The pair of RNA molecules of any one of Embodiments 24-32c, wherein Fragment 3 comprises a nucleotide sequence that is at least 80% identical, such as at least 80%, 85%, 90%, 91%, 92%, 93%, 94%, 95%, 96%, 97%, 98%, 99% or 100% identical, to the nucleotide sequence from the right side of the second interrupted site to the 3′ end of the Group I intron.

Embodiment 32e. The pair of RNA molecules of any one of Embodiments 24-32d, wherein Fragment 3 comprises a nucleotide sequence that is at least 80% identical, such as at least 80%, 85%, 90%, 91%, 92%, 93%, 94%, 95%, 96%, 97%, 98%, 99% or 100% identical, to the nucleotide sequence of SEQ ID NO: 13, 16, 19, 22, 24, 26, 28 or 30.

Embodiment 32f. The pair of RNA molecules of any one of Embodiments 24-32e, wherein Fragment 1, Fragment 2 and Fragment 3 comprise the nucleotide sequence of:
a. SEQ ID NO: 11, SEQ ID NO:12, and SEQ ID NO: 13, respectively;
b. SEQ ID NO: 14, SEQ ID NO:15, and SEQ ID NO: 16, respectively;
c. SEQ ID NO: 17, SEQ ID NO: 18, and SEQ ID NO: 19, respectively;
d. SEQ ID NO: 20, SEQ ID NO: 21, and SEQ ID NO: 22, respectively;
e. SEQ ID NO: 14, SEQ ID NO: 23, and SEQ ID NO: 24, respectively;
f. SEQ ID NO: 14, SEQ ID NO: 25, and SEQ ID NO: 26, respectively;
g. SEQ ID NO: 14, SEQ ID NO: 27, and SEQ ID NO: 28, respectively; or
h. SEQ ID NO: 14, SEQ ID NO: 29, and SEQ ID NO: 30, respectively.

Embodiment 33. The pair of RNA molecules of any one of Embodiments 24-32f, wherein the nucleotide sequence of interest comprises at least one protein-coding sequence and an internal ribosome entry site (IRES) operably linked thereto.

Embodiment 34. The pair of RNA molecules of Embodiment 33, wherein the IRES is directed from any one of the followings: Taura syndrome virus, blood-sucking bug virus, Tyler's encephalomyelitis virus, simian virus 40, red fire ant virus 1, cereal constriction virus, reticulovirus Endothelial hyperplasia virus, Forman poliovirus 1, soybean inchworm virus, Kashmir bee virus, human rhinovirus 2, glass leafhopper virus-1, human immunodeficiency virus type 1, glass leafhopper virus-1, lice P virus, Hepatitis C virus, Hepatitis A virus, Hepatitis GB virus, Foot and mouth disease virus, Human enterovirus 71, Equine rhinovirus, Tea inchworm-like virus, Encephalomyocarditis virus (EMCV), Drosophila C virus, Cruciferous tobacco Virus, cricket paralysis virus, bovine viral diarrhea virus 1, black queen cell virus, aphid lethal paralysis virus, avian encephalomyelitis virus, acute bee paralysis virus, hibiscus yellow ring spot virus, swine fever virus, Aichi virus, kobuvirus, parechovirus, apthovirus, enterovirus, bopivirus, diresapivirus, Aalivirus, Ailurivirus, Ampivirus, Anativirus, Aphthovirus, Avihepatatovirus, Avisivirus, Boosepivirus, Caridiovirus, cosavirus, Crahelivirus, Crohivirus, Dicipivirus, Diresapivirus, entervirus, Eribovirus, Felipivirus, Fipivirus, Galivirus, Grusopivirus, Harkavirus, Hemipivirus, Hepatovirus, Hummivirus, Kunsagivirus, Limnipivirus, Livupivirus, Ludopivirus, Malagasivirus, Megrivirus, Mischivirus, Mosavirus, Mupivirus, Orivirus, parabovirus, parebovirus, pasivirus, passerivirus, pemapivirus, potamipivirus, rabovirus, rafivirus, rohelivirus, rosavirus, sakovuvirus, salivirus, sapelovirus, senecavirus, shanbavirus, sicinvirus, symapivirus, symapivirus, teschovirus, torchivirus, tottorivirus, tremovirus, tropivirus, human FGF2, human SFTPA1, Human AML1/RUNX1, Drosophila Antennae, Human AQP4, Human AT1R, Human BAG-1, Human BCL2, Human BiP, Human c-IAPl, Human c-myc, Human eIF4G, Mouse NDST4L, Human LEF1, Mouse HIFla, Human n.myc, mouse Gtx, human p27kipl, human PDGF2/c-sis, human p53, human Pim-1, mouse Rbm3, Drosophila reaper, canine Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, simian picomavirus, turnip crepe disease virus, eIF4G aptamer, Coxsackie Virus B3 (CVB3) or Coxsackie virus A (CVA1/2). Wild-type IRES sequences can also be modified and used in the present application. Preferably, the IRES is CVB3, BRAV-1L, PV1L, CAV2L, BRAV-1, PV1, or CAV2.

Embodiment 35. The pair of RNA molecules of Embodiment 34, wherein the IRES comprises or consisted of a nucleotide sequence of SEQ ID NO: 51.

Embodiment 36. The pair of RNA molecules of any one of Embodiments 24-35, wherein the nucleotide sequence of interest is a protein coding sequence.

Embodiment 37. The pair of RNA molecules of Embodiment 36, wherein the protein coding region can encode secreted proteins, intracellular proteins, and nuclear proteins in eukaryotic cells.

Embodiment 38. The pair of RNA molecules of Embodiment 37, wherein the protein coding region can encode human proteins, antigens, antibodies, gene editing enzymes such as CRISPR nucleases.

Embodiment 39. The pair of RNA molecules of Embodiment 38, wherein the protein coding region can encode a chimeric antigen receptor, an immunomodulatory protein, or a transcription factor.

Embodiment 40. The pair of RNA molecules of any one of Embodiments 24-35, wherein the nucleotide sequence of interest is a non-protein coding sequence.

Embodiment 41. The pair of RNA molecules of Embodiment 40, wherein the non-protein-coding sequence is selected from antisense RNA, aptamer, guide RNA, or non-protein-coding RNA existing in any organism.

Embodiment 42. The pair of RNA molecules of any one of Embodiments 24-41, wherein the nucleotide sequence of interest is at least 10, 20, 40, 60, 80, 100, 200, 300, 400, 500, 600, 700, 800, 900, 1000, 1500, 2000, 2500, 3000, 3500, 4000, 4500, 10000, 20000 nucleotides in length.

Embodiment 43. The pair of RNA molecules of any one of Embodiments 24-42, wherein the first RNA molecule, second RNA molecule, or permuted RNA molecule comprises a modified nucleotide.

Embodiment 43a. The pair of RNA molecules of Embodiment 43, wherein the modified nucleotide comprises a modified nucleoside.

Embodiment 43b. The pair of RNA molecules of Embodiment 43 or 43a, wherein the modified nucleotide comprises a modified phosphate.

Embodiment 43c. The pair of RNA molecules of any one of Embodiment 43-43b, wherein the modified nucleotide comprises a modified internucleotide linkage.

Embodiment 43d. The pair of RNA molecules of any one of Embodiment 43-43c, wherein the modified nucleotide comprises a cytidine modification, a uridine modification, guanine modification, or an adenosine modification.

Embodiment 43e. The pair of RNA molecules of any one of Embodiment 43-43d, wherein the modified nucleotide includes, but is not limited to, 5-methylcytosine (m5C), N6-methyladenosine (m6A), pseudouridine (ψ), Nlmethylpseudouridine ( [symbol] ), 5-methoxyuridine (5moU), 2′-deoxy-2′-fluoro-ribose (2′-F), a 2′ O-methyl ribose (2′ 0-Me), the acyclic sugar of an UNA nucleotide, a phosphorothioate linkage, or a thiophosphoramidate linkage.

Embodiment 43f. The pair of RNA molecules of any one of Embodiment 43-43e, wherein the first RNA molecule, second RNA molecule, or permuted RNA molecule is chemically modified, partially modified or fully modified; preferably, the permuted RNA molecule comprises at least one nucleotide modification; more preferably, up to 100% of the nucleotides of the permuted RNA molecule are modified.

Embodiment 44. The pair of RNA molecules of any one of Embodiments 43-43f, wherein the at least one nucleotide modification is a cytidine modification, a uridine modification, or an adenosine modification. In some embodiments, the at least one nucleoside modification is selected from the group consisting of 5-methylcytosine (m5C), N6-methyladenosine (m6A), pseudouridine (ψ), Nlmethylpseudouridine ( [symbol] ) and 5-methoxyuridine (5moU).

Embodiment 45. The pair of RNA molecules of Embodiment 44, wherein the permuted RNA molecule comprises less than 100%, less than 90%, less than 80%, less than 70%, less than 60%, less than 50%, less than 40%, less than 30%, less than 20%, less than 15%, less than 10%, less than 5%, less than 1% of a specific nucleotide modification.

Embodiment 46. The pair of RNA molecules of any one of Embodiments 24-42, wherein the first RNA molecule, second RNA molecule, or permuted RNA molecule is unmodified.

Embodiment 47. The pair of RNA molecules of any one of Embodiments 24-46, wherein the first RNA molecule or permuted RNA molecule further comprises a 3′-end binding motif, and the second RNA molecule or Fragment 2(F2) further comprises a 5′-end binding motif, wherein the 3′-end binding motif and 5′-end binding motif are capable of complementary pairing to form a double-stranded region.

Embodiment 47a. The pair of RNA molecules of Embodiment 47, wherein the 3′-end binding motif and the 5′-end binding motif are perfectly complimentary to each other.

Embodiment 48. The pair of RNA molecules of any one of Embodiments 24-47a, wherein the 3′-end binding motif or 5′-end binding motif is about 5-50, about 10-50, about 20-50, about 30-50, about 40-50, about 5-40, about 5-30, about 5-20, or about 10-20 nucleotides in length.

Embodiment 48a. The pair of RNA molecules of Embodiment 48, wherein the 3′-end binding motif or 5′-end binding motif is about 10, 15, or 19 nucleotides in length.

Embodiment 49. A nucleic acid vector for making a circular RNA molecule, wherein the vector encodes the first, second, or permuted RNA molecules of any one of Embodiments 24-42.

Embodiment 50. The nucleic acid vector of Embodiment 49, wherein the vector further comprises an RNA polymerase promoter sequence operably linked to a coding sequence of the first, second, or permuted RNA molecule.

Embodiment 51. The nucleic acid vector of Embodiment 50, wherein the promoter is selected form a T7 RNA polymerase promoter, a T6 viral RNA polymerase promoter, a SP6 viral RNA polymerase promoter, a T3 viral RNA polymerase promoter, or a T4 viral RNA polymerase promoter.

Embodiment 52. The nucleic acid vector of any one of Embodiment 50-51, wherein the vector is selected from a linear DNA fragment, a plasmid vector, a viral vector, a cosmid, bacterial artificial chromosomes (BACs), or yeast artificial chromosomes (YACs).

Embodiment 53. A method for preparing a circular RNA, wherein the method comprises:
i) providing or obtaining the permuted RNA molecule of any one of Embodiments 24-48a by transcribing from the nucleic acid vector of the present application;
ii) annealing the permuted RNA molecule and Fragment 2 in a buffer solution, and then adding GTP and a divalent metal cation at a temperature at which RNA circularization occurs; and
iii) harvesting the circular RNA obtained in step ii).

Embodiment 53a. A method for preparing a circular RNA, wherein the method comprises:
i) providing or obtaining a pair of RNA molecules according to any one of embodiments 24-48a;
ii) adding a buffer solution to the pair of RNA molecules to obtain a reaction mixture to allow the formation of the scaffolding domain and the catalytic domain of the Group I intron, and
iii) adding GTP and a divalent metal cation to the reaction mixture at a temperature to allow the formation of the circular RNA,
optionally, the method further comprises:
iv) harvesting the circular RNA formed in step iii).

Embodiment 54. The method for preparing a circular RNA of Embodiment 53, wherein the permuted RNA molecule and Fragment 2 in a buffer solution are annealed at 1:1; 1:2; 1:3; 1:4; or 1:5 molar ratio, preferably at 1:1 molar ratio.

Embodiment 54a. The method for preparing a circular RNA of Embodiment 53a, wherein the first RNA molecule and the second molecule are provided in the reaction mixture at a 1:1, 1:2, 1:3, 1:4, or 1:5 molar ratio.

Embodiment 55. The method for preparing a circular RNA of any one of Embodiments 53-54a, wherein the buffer solution comprises 10-200 mM Tris-HCl.

Embodiment 55a. The method of Embodiment 55, wherein buffer solution comprises 50 mM Tris-HCl, at pH 6-8.5, such as pH 6, 6.5, 7, 7.5, 8 or 8.5

Embodiment 55b. The method of Embodiment 55a, wherein buffer solution comprises 50 mM Tris-HCl, pH 7.5.

Embodiment 56. The method for preparing a circular RNA of any one of Embodiments 53-55b, wherein the concentration of GTP in the reaction mixture is at the range of 100 nM-2 mM.

Embodiment 57. The method for preparing a circular RNA of any one of Embodiments 53-56, wherein the divalent metal cation is Mg2+, the concentration of the divalent metal cation in the reaction mixture is at least about 5 mM, e.g., about 5 mM to about 550 mM, e.g., at least about 5 mM, about 10 mM, about 15 mM, at least about 20 mM, at least about 30 mM, at least about 40 mM, at least about 50 mM, at least about 60 mM, at least about 70 mM, at least about 80 mM, at least about 90 mM, at least about 100 mM, at least about 125 mM, at least about 150 mM, at least about 175 mM, at least about 200 mM, at least about 250 mM, at least about 300 mM, at least about 350 mM, at least about 400 mM, at least about 450 mM, at least about 500 mM, at least about 550 mM or higher.

Embodiment 58. A circular RNA produced by the method of any one of Embodiments 53-57.

Embodiment 59. A eukaryotic cell comprising the circular RNA of Embodiment 58.

Embodiment 60. A cell population comprising the circular RNA of Embodiment 58.

Embodiment 61. A composition comprising an effective amount of the circular RNA of Embodiment 58 and a pharmaceutically acceptable carrier.

Embodiment 61a. The composition of Embodiment 61, wherein the pharmaceutically acceptable carrier comprises a lipid, a polymer or a lipo-polymeric hybrid, such as a lipid nanoparticle (LNP), a lipid microparticle, a lipid suspension, or a liposome.

Embodiment 62. A composition comprising: a) an effective amount of the circular RNA of Embodiment 58, and b) a delivery vehicle for delivering the circular RNA to a cell, said delivery vehicle comprising a nanocarrier selected from the group consisting of a lipid, a polymer and a lipo-polymeric hybrid.

Embodiment 63. A composition comprising a circular RNA-expressing cell in combination with one or more pharmaceutically or physiologically acceptable carriers, excipients or diluents, wherein the circular RNA is the circular RNA of Embodiment 58.

Embodiment 64. The composition of Embodiment 63, comprising a plurality of circular RNA-expressing cells.

Embodiment 65. The composition of Embodiment 63 or 64, wherein the composition is a nanoparticle formulation.

### EXAMPLES

The present application is further exemplified, but not limited to, by the following examples that illustrate the application.

### Comparative Example: Group I Intron-Mediated RNA Circularization Method

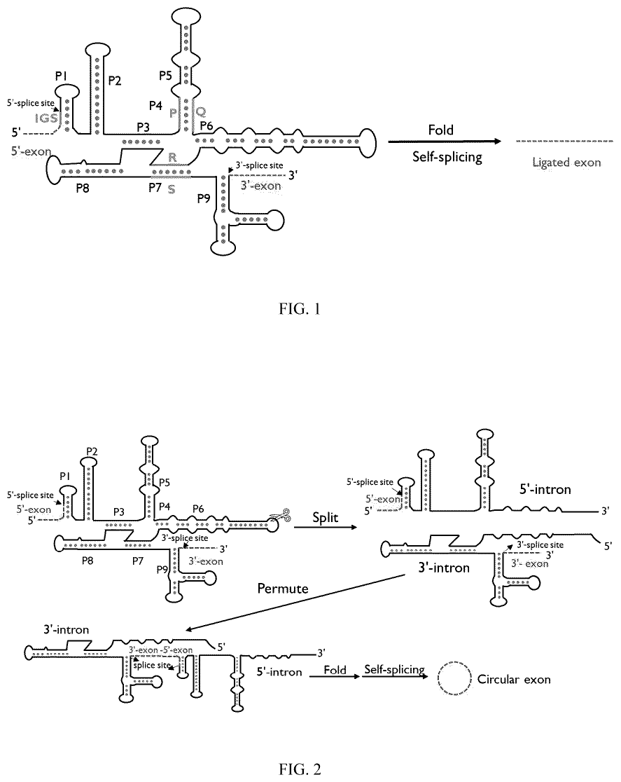

The Group I intron from Anabaena pre-tRNA (SEQ ID: 58), with the secondary structure shown in FIG. 1, is known to in-vitro self-splice most efficiently among the known Group I introns tested. A permuted Group I intron has been reported to be capable of RNA circularization using a PIE (Permute-Intron-Exon) mechanism illustrated in FIG. 2. This PIE splicing circularization method forms a sequence of fused 3′ exon-5′ exon flanked by 3′-intron and 5′-intron, the self-splicing of which results in a circular exon.

### 1.1 Experimental Methods

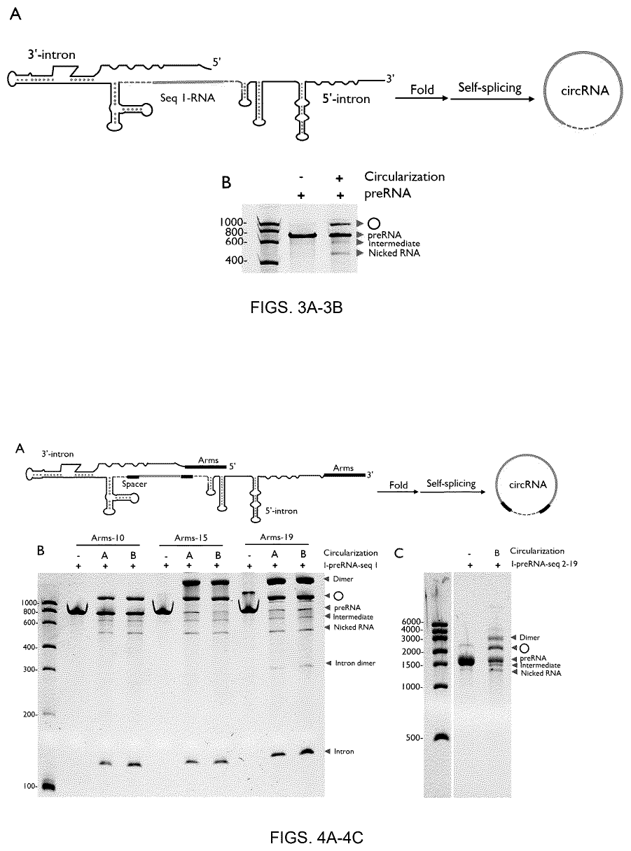

1) Using the PIE strategy as a start point for generating circRNAs with the RNA sequence of interest (FIG. 3). The 3′-intron, 3′ exon, 5′ exon and 5′-intron sequences were used as the scaffold to construct circRNAs comprising seq 1. The seq 1 sequence is shown in SEQ ID: 6 (herein seq1 is only used as a representative example of RNA sequences of interest).
2) The DNA fragment including T7 promoter (SEQ ID: NO. 1), the coding sequences of 3′-intron, 3′ exon, seq 1, 5′ exon, and 5′-intron was cloned into plasmid pUC57, which was completed by Suzhou Genweiz Biotechnology. Plasmid DNA was linearized. The RNA consisted of the 3′-intron (SEQ ID: NO. 2), 3′ exon (SEQ ID: NO. 3), seq 1, 5′ exon (SEQ ID: NO. 4) and 5′-intron (SEQ ID: NO. 5) from 5′ end to 3′ end, which is named preRNA-seq 1, was transcribed in vitro with the linearized plasmid DNA as template. The preRNA-seq 1 was purified by spin column.
3) The purified preRNA-seq 1 was circularized: 1 pg RNA was heated to 70° C. for 5 min and then immediately placed on ice for 3 min, after which GTP was added to a final concentration of 2 mM along with a buffer including magnesium (50 mM Tris-HCl, 10 mM MgCl₂, pH 7.5). RNA was then heated to 52° C. for 10 min, then the circularization reaction was examined by 5% pre-cast denatured UREA-TBE PAGE gel (Wshtbio).
4) Improved PIE method: placing homology arms at the 5′ end and 3′ end of preRNA-seq 1 and inserting spacer sequence between 3′ exon and seq1, between seq 1 and 5′-exon could improve the self-splicing efficiency of PIE (FIG. 4). The improved PIE strategy was reported by Wesselhoeft et al., Nature Communications, 2018, volume 9, Article number: 2629, the content of which is herein incorporated by reference in its entirety. Therefore, 5′-homology arm, 3′-intron, 3′ exon, spacer, spacer, 5′ exon, 5′-intron, 3′-homology arm were used as the scaffold to construct circRNAs containing seq 1. The 5′-homology arm is perfectly paired with the 3′-homology arm. The sequence of seq 1 is shown in SEQ ID: 6.
5) The DNA fragment including T7 promoter, the coding sequences of the 5′-arm in the length of 10, 15, or 19 nt (5′-arm-10/15/19), 3′-intron, 3′ exon, spacer, seq 1, spacer, 5′ exon, 5′-intron, 3′-arm in length 10, 15, or 19 nt (3′-arm-10/15/19) was cloned into plasmid pUC57, which was completed by Suzhou Genweiz Biotechnology.

The plasmid DNA was linearized. The RNA, containing the 5′-arm-10 (SEQ ID: 8) or arm-15 (SEQ ID: 9) or arm-19 (SEQ ID: 10), 3′-intron, 3′ exon, seq 1, 5′ exon, 5′-intron and 3′-arm-10 or 15 or 19 from 5′ end to 3′ end, which is named I-preRNA-seq 1-10 or I-preRNA-seq 1-15 or I-preRNA-seq 1-19, was transcribed in vitro with the linearized plasmid DNA as template. The 5′-arm-10 is perfectly paired with 3′-arm-10. The 5′-arm-15 is perfectly paired with 3′-arm-15. Similarly, the 5′-arm-15 is perfectly paired with 3′-arm-15. The I-preRNA-seq 1-10 or I-preRNA-seq 1-15 or I-preRNA-seq 1-19 was purified by spin column.
6) An RNA expressing seq 2, which has a larger size (herein seq 2 is only used as a representative example of RNA sequences of interest with large size), was circularized by an improved PIE strategy similar to the circularization process for the circRNA expressing seq 1. The RNA contains 5′-arm-19, 3′-intron, 3′ exon, spacer, seq 2, spacer, 5′ exon, 5′-intron and 3′-arm-19 from 5′ end to 3′ end, which is named I-preRNA-seq2-19. The seq 2 sequence is shown in SEQ ID: 7.
7) The purified I-preRNA-seq 1-10, I-preRNA-seq 1-15, I-preRNA-seq 1-19 and I-preRNA-seq 2-19 were circularized in following methods: A-method, 1 pg RNA was heated to 55° C. for 8 min along with 2 mM GTP and a buffer including magnesium (50 mM Tris-HCl, 10 mM MgCl2, 1 mM DTT, pH 7.5). To further improve the splicing efficiency, the optimized B-method was followed: RNA was heated to 85° C. for 5 min and then immediately placed on ice for 3 min, after which GTP was added to a final concentration of 2 mM along with a buffer including magnesium (10 mM), after which RNA was heated to 55° C. for 8 min. Then the circularization reaction (I-preRNA-seq 1-10/15/19) was examined by 5% pre-cast denatured UREA-TBE PAGE gel (Wshtbio) and I-preRNA seq 2-19 was examined by 2% E-gel EX (Invitrogen).

### Results

As shown in FIG. 3B and FIG. 4B, 5% pre-cast denatured UREA-TBE PAGE allows for simple and effective separation of circular splicing products from linear precursor molecules, nicked RNA, splicing intermediates. Since the circular splicing consists of two-step-transesterification reaction, herein the product of intermediates means the products of the first-step transesterification reaction, and the product of nicked RNA is a discontinuity of circRNA molecule where there is no phosphodiester bond between adjacent nucleotides, which means the breakage of circRNAs. The breakage of circRNA named nicked RNA, which shared the same nucleic acid sequence with circRNAs.

For PIE strategy, the self-splicing efficiency is 27.2%, which was assessed by disappearance of the preRNA band and quantitative analysis was conducted by Image J (FIG. 3B). The self-splicing efficiency equation was following:

$$Self - splicing efficiency = ( 1 - \frac{the intensity of preRNA band}{The intensity of ( \begin{matrix}preRNA band + intermediates band + \\ circRNAS band + nicked RNA band + dimer band\end{matrix} )} ) \times 100 \%$$

Meanwhile, the RNA circularization efficiency of the PIE strategy is 19.1%, which was assessed by the appearance of the circRNAs band and quantitative analysis was conducted by Image J (FIG. 3B). The circularization efficiency equation was following:

$$Circularization efficiency = \frac{The intensity of circRNA band}{The intensity of ( \begin{matrix}preRNA band + intermediates RNA + \\ circRNA band + nicked RNA band + dimer band\end{matrix} )} \times 100 \%$$

For improve PIE strategy, the results (FIG. 4B) showed the arm-10 and spacer had contributed to slightly higher RNA splicing efficiency compared to PIE (FIG. 3B), from 27.2% to 29.7%. Meanwhile, the RNA circularization efficiency increased from 19.1% to 22.3%. Through optimizing the splicing condition (Method B), the RNA circularization efficiency of arm-10 and self-splicing efficiency separately reached to 29.4% and 38.8%. The RNA circularization efficiency and self-splicing efficiency were calculated using the above-mentioned equations. However, for longer homology arms arm-15 or arm-19, the preRNA band was obviously decreased but the total circularization efficiency was heavily impaired by the formation of I-preRNA-dimer. The dimer's formation rate was quantified based on the following equation:

$$\frac{The intensity of dimer band}{The intensity of ( \begin{matrix}PreRNA band + intermediates band + \\ circRNA band + nicked RNA band + dimer band\end{matrix} )} \times 100 \%$$

For example, the dimer formation rate of arm-15 reached 58.6% with method A or 54.0% with optimized method B. For arm-19, the dimer formation rate is 63.5% with method A or 55.8% with optimized method B. It showed that the optimized method B could slightly decrease the formation of dimer. The self-splicing efficiency and RNA circularization efficiency of improved PIE were also evaluated using the above equations:

For arm-15, the self-splicing efficiency was 94.3% in Method A or 97.3% in Method B, while the RNA circularization efficiency was 28.8% in Method A or 36.2% in Method B. For arm-19, the self-splicing efficiency was 98.1% in Method A or 97.7% in Method B, while the RNA circularization efficiency was 23.5% in Method A or 27.9% in Method B. It indicated that even long arms would be contributed to high self-splicing efficiency measured by the disappeared of preRNA band, while dimer resulted from long arms significantly impaired the generation of circRNAs.

As the strong homology arms of 19 nucleotides in length contributed to a higher self-splicing efficiency, the 19-nt homology arms were used to synthesize circRNAs with seq 2 (I-preRNA-seq 2-19). The length of I-preRNA-seq 2-19 was about 1.7 kb. As FIG. 4C indicated, with RNA circularization treatment (B-Method), the I-preRNA-dimer band was still clearly shown, suggesting that the 1.7 kb-RNA circularized by the improved PIE also had the problem of forming I-preRNA-dimer.

Accordingly, although the existing improved PIE method could be used to increase the splicing efficiency, it also resulted in the formation of the undesirable dimers.

### Example 1. The Muti-Fragment Assembled Active Group I Intron

In this example, the Group I intron from Anabaena pre-tRNA was divided into three short RNA fragments, self-splicing activity was measured to verify whether the muti-fragments of Group I intron could be assembled into an active intron that undergoes self-splicing.

### 1.1 Experimental Methods

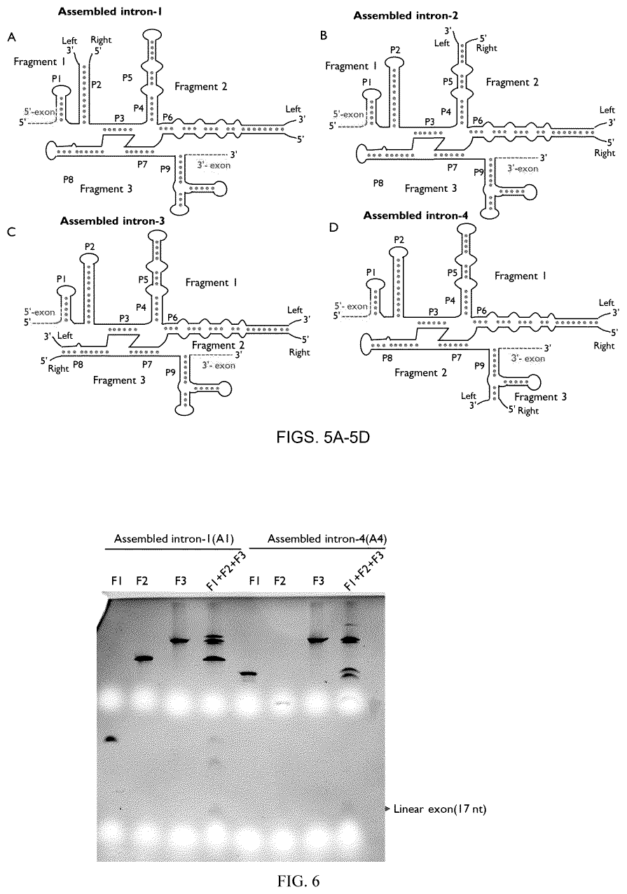

We selected one interrupted site located within the P6 region, and another interrupted site located within the P2 (FIG. 5A), P5 (FIG. 5B), P8 (FIG. 5C) or P9 (FIG. 5D) region of a Group I intron to divide the Group I intron from Anabaena pre-tRNA into three short RNA fragments.

For the assembled intron-1 (FIG. 5A), the resulting fragments were referred to as Fragment 1 (F1), which includes the 5′-splice site, and is from the 5′ end of the Group I Intron to the left side of a first interrupted site located within the P2 region of the Group I intron; Fragment 2 (F2), which is from the right side of the first interrupted site within the P2 region to the left side of a second interrupted site located within the P6 region of the Group I intron; and Fragment 3 (F3), which includes the 3′-splice site, and is from the right side of the second interrupted site located within the P6 region to the 3′ end of the Group I intron.

Similarly, for the assembled intron-2 (FIG. 5B), Fragment 1 includes the 5′-splice site and is from the 5′ end to the left side of a first interrupted site located within the P5 region; Fragment 2 is from the right side of the first interrupted site located within the P5 region to the left side of a second interrupted site located within the P6 region; and Fragment 3 includes the 3′-splice site and is from the right side of the second interrupted site located within the P6 region to the 3′ end of the Group I intron.

For the assembled intron-3 (FIG. 5C), Fragment 1 includes the 5′-splice site and is from the 5′ end of the Group I intron to the left side of a first interrupted site located within the P6 region; Fragment 2 is from the right side of the first interrupted site located within the P6 region to the left side of a second interrupted site located within the P8 region; and Fragment 3 includes the 3′-splice site is from the right side of the second interrupted site located within the P8 region to the 3′ end of the Group I intron.

For the assembled intron-4 (FIG. 5D), Fragment 1 includes the 5′-splice site and is from the 5′ end of the Group I Intron to the left side of a first site located within the P6 region; Fragment 2 is from the right side of the first interrupted site located within the P6 region to the left side of a second interrupted site located within the P9 region; and Fragment 3 includes the 3′-splice site and is from the right side of the second interrupted site located within the P9 region to the 3′ end the Group I intron.

The Fragment 1(F1) and Fragment 3 (F3) could pair with Fragment 2 (F2) to facilitate assembly of the active Group I intron. The assembled introns, such as the assembled intron-1(FIG. 5A), the assembled intron-2 (FIG. 5B), the assembled intron-3(FIG. 5C) and the assembled intron-4 were analyzed to verify the self-splicing activity. The muti-fragments of the intron, e.g., the F1, F2 and F3 of Assembled Intron-1 (AI-1), were solid-phase synthesized by Genscript. The AI-1-F1, AI-1-F2, AI-1-F3 sequence were shown in SEQ ID: 11, 12, 13; The F1, F2 and F3 of Assembled Intron-2 (AI-2), were solid-phase synthesized by Genscript, the AI-2-F1, AI-2-F2, AI-2-F3 sequence were shown in SEQ ID: 14, 15, 16. The F1, F2 and F3 of Assembled Intron-3 (AI-3), were solid-phase synthesized by Genscript, the AI-3-F1, AI-3-F2, AI-3-F3 sequence were shown in SEQ ID: 17, 18, 19. And the F1, F2 and F3 of Assembled Intron-4(AI-4), were solid-phase synthesized by Genscript, the AI-4-F1, AI-4-F2, AI-4-F3 sequence were shown in SEQ ID: 20, 21, 22.

The muti-fragment intron was assembled by F1, F2 and F3 and then self-splicing. In particular, 50 pmol AI-1-F1/AI-2-F1/AI-3-F1/AI-4-F1, 50 pmol AI-1-F2/AI-2-F2/AI-3-F2/AI-4-F2, and 50 pmol AI-1-F3/AI-2-F3/AI-3-F3/AI-4-F3 were incubated at 37° C. for 1 h in a buffer (50 mM pH 7.5 Tris-HCl). After the incubation, GTP and MgCl₂ were added to the final concentration of 2 mM and 34 mM, respectively, and then further incubated 37° C. for 30 min. The self-splicing reaction was analyzed by 20% pre-cast denatured UREA-TBE PAGE gel (Wshtbio).

### Results

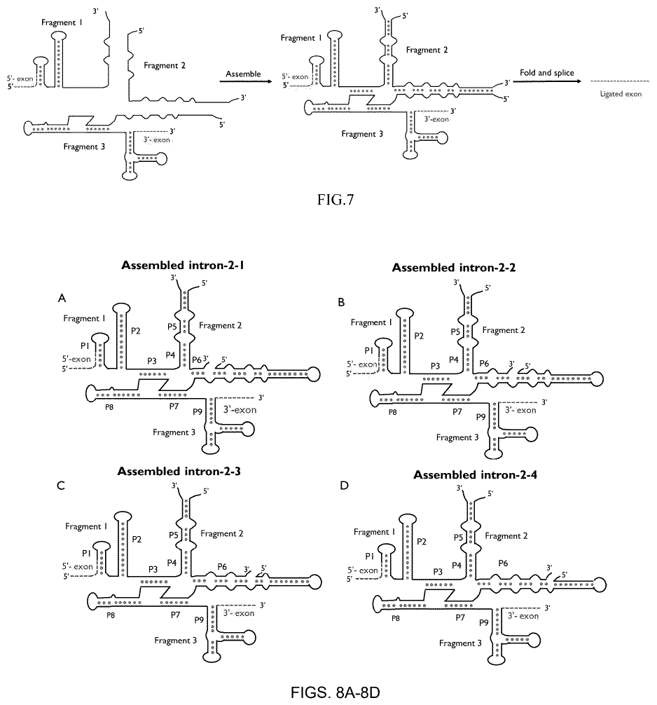

The results showed that the exon band (17 nt), the self-splicing product of Group I intron, appeared after AI-1-F1, AI-1-F2 and AI-1-F3 assembled to muti-fragment active Group I intron, and AI-4-F1, AI-4-F2 and AI-4-F3 assembled to muti-fragment active Group I intron, which verified the self-splicing activity of muti-fragment Group I intron. The position of exon (the product of splicing of muti-fragment Group I intron) in the gel was indicated by the red arrow (FIG. 6).

In summary, the Group I intron can split three parts (Fragment 1(F1), Fragment 2(F2) and Fragment 3(F3)), and then these three parts can recombine together to make up a multi-fragment Group I intron, which can self-splice to synthesize exon as FIG. 7.

### Example 2. Trans-Splicing Strategy to Synthesize Circular Exon

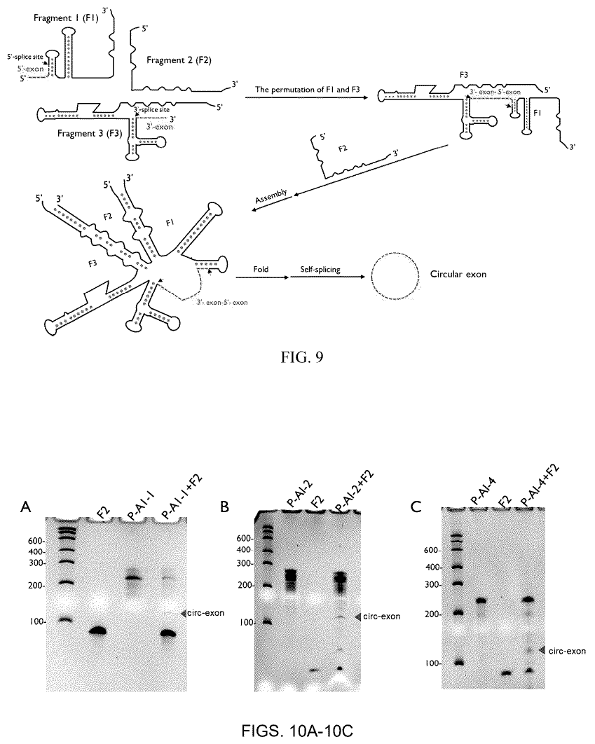

This is an exemplary illustration of using a method involving multi-fragment ribozymes to generate a circular RNA. Specifically, an RNA containing Fragment 1 (F1), 5′ exon, Fragment 3 (F3) and 3′ exon was permutated to obtain a first RNA molecule (permutated RNA molecule) containing F3-3′ exon-5′ exon-F1. The first RNA molecule was self-spliced to generate a circular exon with the help of Fragment 2 (F2). This method of generating a circular exon is named as trans-splicing strategy (FIG. 9).

### 2.1 Experimental Methods:

To generate the circular exon, a DNA fragment containing T7 promoter operably linked to a coding sequence of F3-3′ exon-5′ exon-F1 was synthesized by PCR, primers were obtained from Suzhou Genweiz Biotechnology. The DNA fragment was subject to in vitro transcription to product the permutated RNA molecule containing F3-3′ exon-5′ exon-F1. A second RNA molecule containing Fragment 2 (F2) was chemically synthesized by Genscript. The permuted RNA molecule and the second RNA molecule were derived from each of the Assembled Intron-1 to Intron-4. The activity in generating circular exon from each of the permutated RNA molecule with the help of the corresponding F2 sequence was measured. For Assembled Intron-1 (AI-1, FIG. 5A), the Fragment 3 (F3) sequence was shown in SEQ ID NO: 13, the F2 sequence was shown in SEQ ID NO:12, the Fragment 1 (F1) sequence was shown in SEQ ID NO:11. The sequence of permutated RNA molecule of Assembled Intron-1(P-AI-1) was shown in SEQ ID NO: 31. For Assembled Intron-2 (AI-2, FIG. 5B), the F3 sequence was shown in SEQ ID NO: 16, the F2 sequence was shown in SEQ ID NO:15, the F1 sequence was shown in SEQ ID NO:14. The sequence of permutated RNA molecule of Assembled Intron-2(P-AI-2) was shown in SEQ ID NO: 32. For Assembled Intron-3 (AI-3, FIG. 5C), the F3 sequence was shown in SEQ ID NO: 19, the F2 sequence was shown in SEQ ID NO:18, the F1 sequence was shown in SEQ ID NO:17. The sequence of permutated RNA molecule of Assembled Intron-3(P-AI-3) was shown in SEQ ID NO: 33. For Assembled Intron-4 (AI-4, FIG. 5D), the F3 sequence was shown in SEQ ID NO: 22, the F2 sequence was shown in SEQ ID NO:21, the F1 sequence was shown in SEQ ID NO:20. The sequence of permutated RNA molecule of assembled intron-4(P-AI-3) was shown in SEQ ID NO: 34. The influence of the different splitting site on P6 coupled with the splitting site at P5 on generating circular exon was also analyzed. The details of splitting were illustrated in FIGS. 8A-D. The splits were separately named as Assembled Intron 2-1(AI-2-1, FIG. 8A), Assembled Intron 2-2(AI-2-2, FIG. 8B), Assembled Intron 2-3(AI-2-3, FIG. 8C), and Assembled Intron 2-4(AI-2-4, FIG. 8D), respectively. The permuted RNA molecule was used to generate circular exon as the above mentioned. For assembled intron 2-1(AI-2-1), the F2 sequence was shown in SEQ ID NO: 23, the F3 sequence was shown in SEQ ID NO: 24. The sequence of permutated RNA molecule of AI-2-1 (P-AI-2-1) was shown in SEQ ID NO: 35. For Assembled Intron 2-2 (AI-2-2), the F2 sequence was shown in SEQ ID NO: 25, the F3 sequence was shown in SEQ ID NO: 26. The sequence of permutated RNA molecule of assembled AI-2-2 (P-AI-2-2) was shown in SEQ ID NO: 36. For Assembled Intron 2-3(AI-2-3), the F2 sequence was shown in SEQ ID NO: 27, the F3 sequence was shown in SEQ ID NO: 28. The sequence of permutated RNA molecule of AI-2-3 (P-AI-2-3) was shown in SEQ ID NO: 37. For Assembled Intron 2-4 (AI-2-4), the F2 sequence was shown in SEQ ID NO: 29, the F3 sequence was shown in SEQ ID NO: 30. The sequence of permutated RNA molecule of AI-2-4 (P-AI-2-4) was shown in SEQ ID NO: 38. The assembled intron 2-1/2/3/4 shared F1 as showed in SEQ ID NO 14.

The DNA fragment was synthesized by Polymerase Chain Reaction (PCR) using primers obtained from Suzhou Genweiz Biotechnology. The RNA containing F3, 3′ exon, 5′ exon, and F1 from the 5′ end to 3′ end, which were named Permuted assembled intron-1(P-AI-1), Permuted assembled intron-2 (P-AI-2), Permuted assembled intron-3(P-AI-3), Permuted assembled intron-4(P-AI-4), Permuted assembled intron-2-1(P-AI-2-1), Permuted assembled intron-2-2(P-AI-2-2), Permuted assembled intron-2-3(P-AI-2-3) and Permuted assembled intron-2-4(P-AI-2-4) were transcribed in vitro with the—DNA fragment obtained by PCR as template. The P-AI-1/P-AI-2/P-AI-3/P-AI-4/P-AI-2-1/P-AI-2-2/P-AI-2-3/P-AI-2-4 RNA was purified by spin column. The circ-exon, the product of trans-splicing strategy, was shown in SEQ ID NO: 39.

The column-purified P-AI-1/P-AI-2/P-AI-3/P-AI-4/P-AI-2-1/P-AI-2-2/P-AI-2-3/P-AI-2-4 was subject to the circularization treatment with the help of its corresponding F2. Specifically, the P-AI-1/P-AI-2/P-AI-3/P-AI-4/P-AI-2-1/P-AI-2-2/P-AI-2-3/P-AI-2- and its corresponding F2 at 1:1 molar ratio was annealed in a buffer (50 mM Tris-HCl, pH 7.5). GTP and MgCl₂ were added to the annealed system (the final concentration of GTP is 2 mM, MgCl₂ is 34 mM), which was then heated to 42° C. for 10 min. The trans-circularization reaction system 1contains the reagents shown in Table 1.

TABLE 1
Trans-circularization reaction system 1

| Reagent | Volume |
|---|---|
| 50 pmol P-AI-1 | 1 μL |
| 50 pmol F2 | 1 μL |
| 10 X buffer | 1 μL |
| 20 mM GTP | 1 μL |
| 340 mM MgCl₂ | 1 μL |
| H₂O | 5 μL |
| | Total, 10 μL |

The circularization reaction products were examined by 5% pre-cast denatured UREA-TBE PAGE gel (bought from Wshtbio) with 180 V, 45 min.

### Results

To investigate the possibility of generating a circular exon (circ-exon) by trans-splicing strategy, we used the P-AI-1 and its corresponding F2, which derived from Assembled Intron-1, to generate circular exon. As the data (FIG. 10A) showed the circ-exon band was clearly present in the PAGE gel (marked by a red arrow). Similarly, circ-exon was generated by P-AI-2 and its corresponding F2 (derived from Assembled Intron-2), P-AI-4 and its corresponding F2 (derived from assembled intron-4), as indicated in FIG. 10B and FIG. 10C. In addition, we needed to mention the purity of in-vitro transcribed RNA, P-AI-1, P-AI-2 and P-AI-4 is poor.

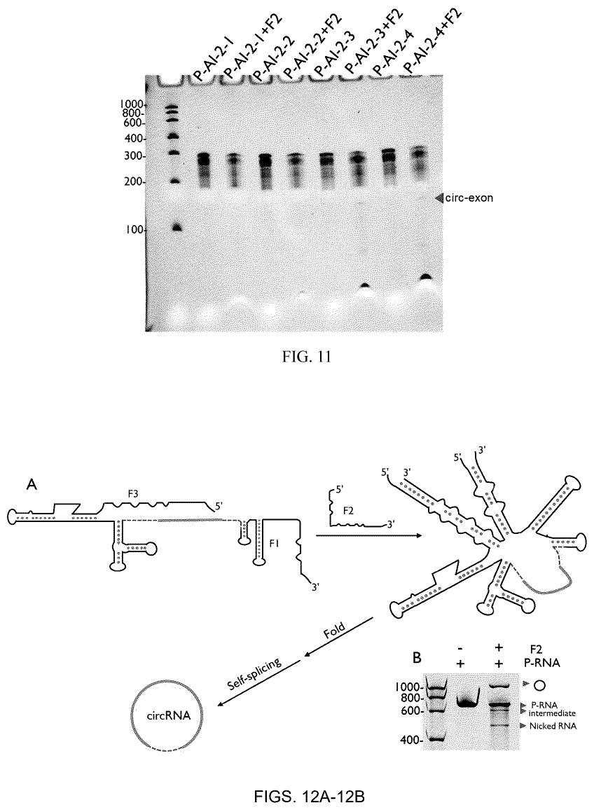

Furthermore, we test the influence of the different splitting site on P6 coupled with the splitting site at P5 on generating circ-exon, that is the efficiency of P-AI-2-1, P-AI-2-2, P-AI-2-3 and P-AI-2-4 with the help of its corresponding F2 generating circ-exon. It was found that the P-AI-2-3 with the help of its corresponding F2 and P-AI-2-4 with the help of its corresponding F2 could generating circ-exon indicated by red arrow in FIG. 11.

Briefly, the above data indicated the trans-splicing strategy could be used to generate circ-exon.

### Example 3. Trans-Splicing Strategy to Synthesize circRNAs with RNA Sequence of Interest

In this example, circRNAs with an RNA sequence of interest were produced using a trans-splicing strategy shown in FIG. 12A.

### 3.1 Experimental Methods

To verify the trans-splicing strategy for generating circRNA with sequence of interest, Fragment (F3) including a 3′ splicing site, 3′ exon, 5′ exon, and Fragment 1 (F1) including a 5′ splicing site, were used as the scaffold to assemble a permuted RNA molecule for synthesizing a circRNA with an RNA sequence of interest (herein seq 1 is used as a representative example of RNA sequence of interest). The seq 1 sequence was shown in SEQ ID: 6, which is the same as that in the Comparative Example. The DNA was synthesized by Suzhou Genweiz Biotechnology. The fragment 2 (F2) was synthesized by Genscript. The F1 sequence was the same as that in Example 2 shown in SEQ ID NO: 14. The F2 sequence was the same as that in Example 2 shown in SEQ ID NO: 15, and the F3 sequence was the same as that in Example 2 shown in SEQ ID NO: 16.

The DNA fragment including T7 promoter, and the coding sequences of F3, 3′ exon, seq 1, 5′ exon, and F1 was cloned into plasmid pUC57, which was completed by Suzhou Genweiz Biotechnology. Plasmid DNA was linearized. The RNA containing F3, 3′ exon, seq 1, 5′ exon, F1 from 5′ end to 3′ end, which was named Permuted-RNA-seq 1 (P-RNA-seq 1), was transcribed in vitro with the linearized plasmid DNA as template. The P-RNA-seq 1 RNA was purified by spin column. The P-RNA-seq 1 sequence was shown in SEQ ID NO: 40.

The purified P-RNA-seq 1 was subject to the circularization treatment with the help of F2. Specifically, the P-RNA-seq 1 and F2 at 1:1 molar ratio was annealed in a buffer (50 mM Tris-HCl, pH 7.5). GTP and MgCl₂ were added to the annealed system (the final concentration of GTP is 2 mM, MgCl₂ is 34 mM), which was then heated to 52° C. for 10 min. The trans-circularization reaction system 2 contains the reagents shown in Table 2.

TABLE 2
Trans-circularization reaction system 2

| Reagent | Volume |
|---|---|
| 50 pmol P-RNA-seq 1 | 1 μL |
| 50 pmol F2 | 1 μL |
| 10 X buffer | 1 μL |
| 20 mM GTP | 1 μL |
| 340 mM MgCl₂ | 1 μL |
| H₂O | 5 μL |
| | Total, 10 μL |

The circularization reaction products were examined by 5% pre-cast denatured UREA-TBE PAGE gel (bought from Wshtbio) with 180 V, 45 min.

### 3.2 Results

The results showed the circRNAs band clearly appeared after the circularization treatment with the help of F2 (FIG. 12B), demonstrating that the trans-splicing strategy could be used to generate circRNAs. The splicing efficiency of trans-splicing strategy was about 32.3%, which was assessed by the disappearance of the P-RNA-seq 1 band and the quantitative analysis was conducted by Image J. The splicing efficiency was calculated according to the equation referred to above in the Comparative Example.

The RNA circularization efficiency was 16.8%, which was measured by the appearance of the circRNAs band and the quantitative analysis was conducted by Image J (FIG. 12B). The circularization efficiency equation was calculated according to the equation referred to above in Comparative Example.

In conclusion, the trans-splicing strategy according to an embodiment of the application had a splicing activity which could be used to synthesize circRNAs with an RNA sequence of interesting.

### Example 4. Improved Trans-Splicing Strategy to Synthesize circRNAs with an RNA Sequence of Interest

A perfectly complementary “binding motif” was placed at the 3′ end of P-RNA-seq 1 (which is named as Improved Permuted RNA seq 1, or I-P-RNA-seq 1), and the 5′ end of Fragment 2 (named BM-F2). The assembly of I-P-RNA-seq 1 and BM-F2 was analyzed. The results showed that, with the help of BM-F2, the well folded active complex, I-P-RNA-seq 1 assembled with BF-M2, had higher efficiency of self-splicing and higher efficiency in generating circRNAs (FIG. 13A).

### 4.1 Experimental Methods

To test the possibility of improved trans-splicing strategy, the Fragment (F3), 3′ exon, 5′ exon, Fragment 1 (F1), binding motif sequence in the length of 5, 10, 15, and 20, 50 nt were used as the scaffold to construct circRNAs expressing seq 1. The seq 1 sequence was shown in SEQ ID: 6. The DNA was synthesized by Suzhou Genweiz Biotechnology. Fragment 2 (F2) with the binding motif in length 5, 10, 15, 20 and 50 nt (BM(5/10/15/20/50)-F2) at 5′ end was chemically synthesized by Genscript. The BM (5)-F2, BM (10)-F2, BM (15)-F2, BM (20)-F2 and BM (50)-F2 sequence were separately shown in SEQ ID NOs: 41, 42, 43, 44, 45.

The DNA fragment including T7 promoter, F3, 3′ exon, seq 1, 5′ exon, F1 and binding motif in the length of 5, 10, 15, 20 and 50 nt (BM-5/10/15/20/50) was cloned into plasmid pUC57, which was conducted by Suzhou Genweiz Biotechnology. Plasmid DNA was linearized. The RNA containing F3, 3′ exon, seq 1, 5′ exon, F1 and BM-5/10/15/20/50 from 5′ end to 3′ end, which was named I (5/10/15/20/50)-P-RNA-seq 1, was transcribed in vitro with the linearized plasmid DNA as template. The I (5/10/15/20/50)-P-RNA-seq 1 was purified by spin column. The I (5)-P-RNA-seq 1, I (10)-P-RNA-seq 1, I (15)-P-RNA-seq 1, I (20)-P-RNA-seq 1, and I (50)-P-RNA-seq 1 sequence were separately shown in SEQ ID NO: 46, 47, 48, 49, 50.

The purified I (5/10/15/20/50)-P-RNA-seq 1 was subjected to the circularization treatment with the help of BM (5/10/15/20/50)-F2. In particular, the I (5/10/15/20/50)-P-RNA-seq 1 and BM (5/10/15/20/50)-F2 at 1:1 molar ratio were annealed in a buffer (50 mM Tris-HCl, pH 7.5). GTP and MgCl₂ were added to the annealed system (the final concentration of GTP is 2 mM, MgCl₂ is 34 mM), which was then heated to 52° C. for 10 min. The trans-circularization reaction system 3 contains the reagents shown in Table 3.

TABLE 3
Trans-circularization reaction system 3

| Reagent | Volume |
|---|---|
| 50 pmol I-P-RNA-seq 1 | 1 μL |
| 50 pmol BM-F2 | 1 μL |
| 10 X buffer | 1 μL |
| 20 mM GTP | 1 μL |
| 340 mM MgCl₂ | 1 μL |
| H₂O | 5 μL |
| | Total, 10 μL |

Meanwhile, we optimized the trans-circularization reaction to improve the circularization efficiency of improved trans-splicing strategy. And then the circularization reaction products were examined by 5% pre-cast denatured UREA-TBE PAGE gel (bought from Wshtbio) with 180 V, 45 min.

To verify that the circularization product was circRNA, the circularization reaction was treated with RNase R. The composition of a typical 20 μL reaction mixture contained: 1 μg RNA, 20 mM Tris-HCl (pH 8.0), 10 mM NaCl, 0.1 mM MgCl2, 1 U/2U/3U RNase R (Beyotime). The reaction mixture was incubated at 37° C. for 30 min, then was heat to 70° C. for 10 min to inactive RNase R. The RNase R-digested RNA was then analyzed by 5% pre-cast denatured UREA-TBE PAGE gel.

The I-P-RNA-seq 1 subjected to circularization treatment was reverse transcribed (RT) into cDNA using a Hiscript 1^{st} strand cDNA synthesis kit with random primers (vazyme, R111-02) according to manufacturer's instructions. One-tenth of the RT product was used for PCR amplification in real-time PCR system with divergent primers, which could only amplify transcripts across the splice junction of circRNAs. The PCR products were analyzed by 1% agarose gel. The PCR products were also cloned into plasmids by TA cloning for sequencing (Shanghai Sangon).

### 4.2 Results

Firstly, the effect of the length of binding motif on circularization efficiency of trans-splicing strategy was investigated. As FIG. 13B displayed all tested binding motifs (5 nt, 10 nt, 15 nt and 20 nt) can promote to generate circRNA, especially the length of binding motif is 10 nt, 15 nt and 20 nt. Although the 50 nt binding motif also can facilitate trans-splicing strategy mediated circRNA generation, the partly assembled BM (50)-F2 and I (50)-P-RNA-seq 1 (marked by yellow star in FIG. 13B) decreased the proportion of circRNA.

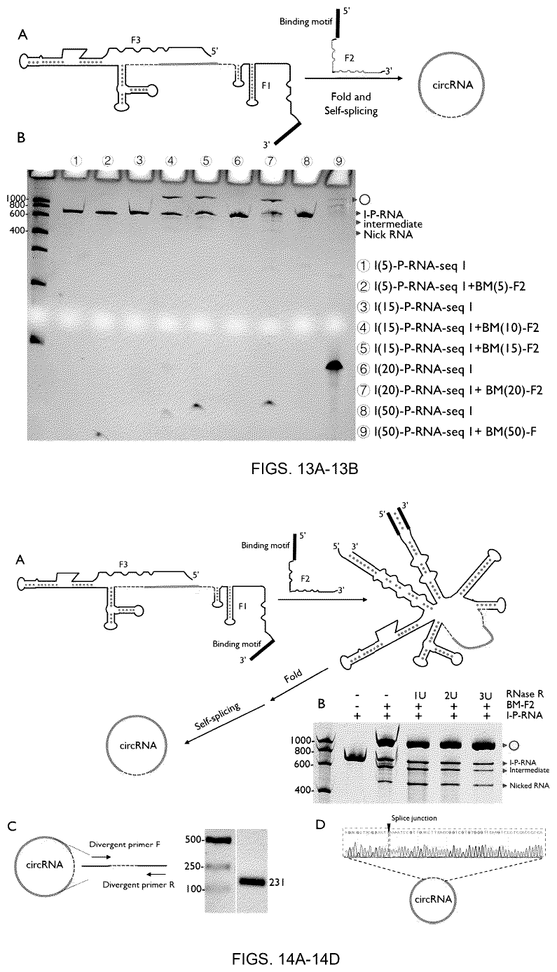

Moreover, after optimizing the condition of circularization reaction, the RNA circularization efficiency of improved trans-splicing strategy was examined. The results (FIG. 14B) showed adding the binding motif to F2 (BM(10)-F2) and P-RNA-seq 1(I—P-RNA-seq 1) increased the splicing efficiency of trans-splicing strategy from 32.3% (FIG. 12B) to 77.9% (the third lane of FIG. 14B, the first lane of FIG. 14B was RNA ladder), which was assessed by the disappearance of the I-P-RNA-seq 1 and the quantitative analysis was conducted by Image J. In addition, this improved method increased the RNA circularization efficiency of trans-splicing strategy from 16.8% (FIG. 12B) to 57.2% (FIG. 14B). Most importantly, no dimer has been discovered using this improved method. The splicing efficiency and RNA circularization efficiency were each calculated according to the respective equations referred to in the Comparative Example. Accordingly, the improved trans-splicing strategy according to an embodiment of the application, has not only improved the RNA circularization efficiency, but also resulted in no detectable dimer formation, which is surprising as compared to the existing improved PIE (FIG. 4B).

To ensure that the major circularization product of the improved trans-splicing strategy was indeed circRNA, the circularization reaction was treated with different units of RNase R (1U, 2U or 3 U). CircRNAs resistant to RNase R were confirmed by observing a clear band of circRNA even after the RNase R treatment, whereas the linear products were partially or totally degraded by the RNase R treatment (FIG. 14B lane 4, lane 5 and lane 6).

It is well known that divergent primers designed to span the circRNAs splice junction sequence can specifically amplify the circRNAs but not the counterpart linear RNA. The PCR products in 231-bp size amplified by divergent primers was analyzed by 1% agarose gel. The results in FIG. 14C showed that the aimed band of PCR products (231 bp) have exhibited the expected size. In addition, the sequencing result of PCR products validated the correct splice junction of the circRNAs (FIG. 14D).

In conclusion, this example demonstrated that circRNAs was successfully generated via the improved trans-splicing strategy according to an embodiment of the application, with higher RNA circularization efficiency and no detectable by-products of dimers.

### Example 5. CircRNAs Synthesized by the Improved Trans-Splicing Method can Express Secreted Protein Gaussia Luciferase in 293T Cells

### 5.1 Experimental Methods

The EMCV IRES, F3, 3′ exon, 5′ exon, F1, binding motif sequence were used as the basic elements to construct circRNAs expressing secreted protein Gaussia luciferase (Gluc).

The EMCV IRES sequence is shown in SEQ ID NO: 51. The Gluc sequence is shown in SEQ ID NO: 52. The DNA was synthesized by Suzhou Genweiz Biotechnology. BM-F2 was chemically synthesized by Genscript. The BM-F2 sequence was the same as that used in Example 4.

A DNA fragment including T7 promoter, F3, 3′ exon, EMCV IRES, Gluc coding region, 5′ exon, F1, binding motif was cloned into plasmid pUC57, which was completed by Suzhou Genweiz Biotechnology. Plasmid DNA was linearized, the RNA (named I-P-RNA-Glu) including F3, 3′ exon, EMCV IRES, Gluc, 5′ exon, F1 and Binding motif was transcribed in vitro with the linearized plasmid DNA as template. The I-P-RNA-Glu (SEQ ID NO: 53) was purified, subject to circularization treatment with the help of BM-F2, and then was purified again, using a procedure described in Example 4.

To enrich for circRNA, 20 μg of RNA was diluted in water (86 μL) and then heated at 65° C. for 3 min and cooled on ice for 3 min. 30U RNase R and 10 μL of 10× RNase R buffer (Beyotime) was added, and the reaction was incubated at 37° C. for 15 min and then was heated to 70 min for 10 min. The RNase R-digested RNA was column purified.

To evaluate translation efficiency of enriched circRNA-Gluc in 293T cells, 293T cells were first seeded in a 96-well plate at 1×10⁴ cells/well and cultured in 37° C. with 5% CO₂. After cells adhesion, Lipofectamine MessengerMax (Invitrogen) transfection reagent was used to transfect 200 ng-RNase R digested RNA per well, or to transfect 200 ng-RNase R digested RNA expressing eGFP-NLS (circRNA-eGFP-NLS) as control.

The transfected cells were analyzed by Gaussia luciferase glow assay. In particular, transfected cells were incubated for 24 hours at 37° C. in 5% CO₂ in a cell incubator and then 10 μL of medium was removed from the transfected cells. The luminometer was programmed. The 10 μL collected medium was added to a black 96-well plate, and 50 μL of Working Solution (containing Coelenterazine) was then added to each well. After 10 minutes for signal stabilization, the light output was detected.

### 5.2 Results

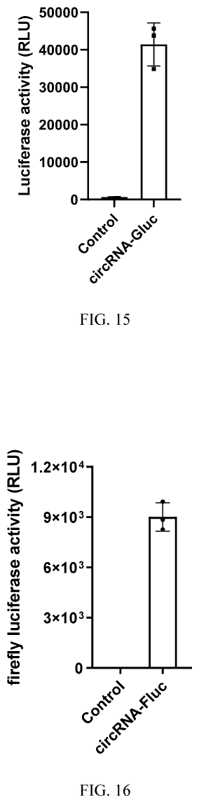

The medium from 293 T transfected with circRNAs-Gluc was assayed for Gluc activity using the Pierce Gluc Glow Assay Kits. The Gluc activity was shown in FIG. 15. Compared to the control (293 T cells transfected circRNA-eGFP-NLS), 293 T cells transfected circRNAs-Gluc resulted in robust production of secreted protein Gluc.

### Example 6. CircRNAs Synthesized by the Improved Trans-Splicing Method can Express Intracellular Protein Firefly Luciferase in 293T Cells

### 6.1 Experimental Methods

The EMCV IRES, F3, 3′ exon, 5′ exon, F1, binding motif sequence were used as the basic elements to construct circRNAs expressing intracellular protein firefly luciferase (Fluc). The Fluc sequence was shown in SEQ ID NO: 54. The DNA was synthesized by Suzhou Genweiz Biotechnology. BM-F2 was chemically synthesized by Genscript. The BM-F2 was the same as that used in Example 4.

A DNA fragment including T7 promoter, F3, 3′ exon, EMCV IRES, Fluc coding region, 5′ exon, F1, binding motif was cloned into plasmid pUC57, which was completed by Suzhou Genweiz Biotechnology. Plasmid DNA was linearized, the RNA (named I-P-RNA-Fluc) including F3, 3′ exon, EMCV IRES, Fluc, 5′ exon, F1 and binding motif was transcribed in vitro with the linearized plasmid DNA as template. The I-P-RNA-Fluc (SEQ ID: 55) was purified, subject to circulation treatment with the help of BM-F2, and then was purified again, using the procedure described in Example 4.

The circRNA-Fluc was enriched and transfected into 293T cell using procedures described in Example 5.1. The control cells were transfected with 200 ng-RNase R digested RNA expressing eGFP-NLS (circRNA-eGFP-NLS).

The transfected cells were analyzed using a firefly luciferase assay. In particular, the transfected cells were incubated for 24 hours at 37° C. in 5% CO₂ in a cell incubator. The culture medium was aspirated and the cells were rinsed with 100 μL/well of 1×DPBS buffer (Thermo fish). After the DPBS was aspirated, 100 μL/well 1× cell Lysis buffer was added to the cells. The plate was shaken on a platform shaker at moderate speed for 15 minutes. The cell lysis was transferred to a black 96-well plate and an equal volume Working Solution (containing substrate D-Luciferin) was added to each well. After 10 minutes for signal stabilization, the light output was detected.

### 6.2 Result

Cell lysis from 293 T cells transfected with circRNAs-Fluc was assayed for Fluc activity using the Pierce Fluc Glow Assay Kits. The Fluc activity was shown in FIG. 16. Compared to the control (293 T cells transfected circRNA-eGFP-NLS), 293 T cells transfected circRNAs-Fluc resulted in robust production of intracellular protein Fluc.

### Example 7. CircRNAs Synthesized by the Improved Trans-Splicing Method can Express Intranuclear Protein in 293T Cells

### 7.1 Experimental Methods

The EMCV IRES, F3, 3′ exon, 5′ exon, F1, Binding motif sequence were used as the basic elements to construct circRNAs expressing intranuclear protein. The fluorescent protein eGFP fused to Nuclear Localization Signal (NLS) was used to mimic intranuclear protein. The eGFP-NLS sequence is shown in SEQ ID NO: 56. The DNA was synthesized by Suzhou Genweiz Biotechnology. BM-F2 was the same as that in Example 4.

A DNA fragment including T7 promoter, F3, 3′ exon, EMCV IRES, eGFP-NLS coding sequence, 5′ exon, F1, binding motif was cloned into plasmid pUC57, which was conducted by Suzhou Genweiz Biotechnology. Plasmid DNA was linearized, the RNA (named I-P-RNA-eGFP-NLS) including F3, 3′ exon, EMCV IRES, eGFP-NLS, 5′ exon, F1 and binding motif was transcribed in vitro with the linearized plasmid DNA as template. The I-P-RNA-eGFP-NLS (SEQ ID: 57) was purified, was circularized, and then was purified again, as the procedure mentioned in Example 4.

The circRNA-eGFP-NLS was enriched using a procedure as described in Example 5.1.

The 293T cells were transfected with the circRNA-eGFP-NLS using a procedure described in Example 5.1, except seeding 20,0000 cells/well in 12-well plate.

The transfected cells were analyzed by an eGFP-NLS assay. In particular,
the transfected cells were incubated for 24 hours at 37° C. in 5% CO₂ in a cell incubator. Then, 10 μL 100× Hoechst 33342 (Beyotime) living cell dye was added to transfected cells containing 1 mL media, followed by incubation at 37° C. in 5% CO₂ in a cell incubator for 10 min. The culture medium was aspirated from the cells. The cells were then rinsed with DPBS buffer twice. Thereafter, the cells were observed with a fluorescence microscope in 20-fold magnification.

### 7.2 Result

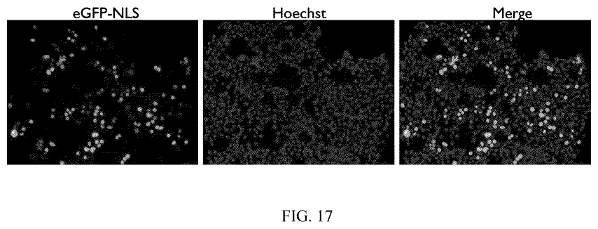

The 293 T cells transfected with circRNA-eGFP-NLS were observed with a fluorescence microscope. To verify the eGFP-NLS expressed by circRNAs was in nuclear, Hoechst, a nucleus dye, was used to indicate the location of nuclear. As shown in FIG. 17, eGFP-NLS is co-located with nucleus dyed by Hoechst, which indicated eGFP-NIS expressed by circRNAs could relocate exactly to nuclear.

### Sequence Information

| SEQ ID | | |
|---|---|---|
| NO. | Sequence | Description |
| 1 | TAATACGACTCACTATAGG | T7 |
| | | promotor |
| 2 | ACAACAGAUAACUUACAGCUAGUCGGAAGGUGCAG | 3′-intron |
| | AGACUCGACGGGAGCUACCCUAACGUCAAGACGAG | (PIE |
| | GGUAAAGAGAGAGUCCAAUUCUCAAAGCCAAUAGG | method) |
| | CAGUAGCGAAAGCUGCGGGAGAAUG | |
| 3 | AAAAUCCGUUGACCUUAAACGGUCGUGUGGGUUCA | 3′ exon |
| | AGUCCCUCCACCCCCA | (PIE |
| | | method) |
| 4 | uggcggaaugguAGACGCUACGGACUU | 5′ exon |
| | | (PIE |
| | | method) |
| 5 | AAAUAAUUGAGCCUUAGAGAAGAAAUUCUUUAAGU | 5′-intron |
| | GGAUGCUCUCAAACUCAGGGAAACCUAAAUCUAGC | (PIE |
| | UAUAGACAAGGCAAUCCUGAGCCAAGCCGAAGUAG | method) |
| | UAAUUAGUAAGUUA | |
| 6 | CCCCUCUCCCUCCCCCCCUAACGUUACUGGCCGAAG | Seq 1 |
| | CCGCUUGGAAUAAGGCCGGUGUGCGUUUGUCUAUA | |
| | UGUUAUUUUCCACCAUAUUGCCGUCUUUUGGCAAU | |
| | GUGAGGGCCCGGAAACCUGGCCCUGUCUUCUUGAC | |
| | GAGCAUUCCUAGGGGUCUUUCCCCUCUCGCCAAAG | |
| | GAAUGCAAGGUCUGUUGAAUGUCGUGAAGGAAGCA | |
| | GUUCCUCUGGAAGCUUCUUGAAGACAAACAACGUC | |
| | UGUAGCGACCCUUUGCAGGCAGCGGAACCCCCCACC | |
| | UGGCGACAGGUGCCUCUGCGGCCAAAAGCCACGUG | |
| | UAUAAGAUACACCUGCAAAGGCGGCACAACCCCAG | |
| | UGCCACGUUGUGAGUUGGAUAGUUGUGGAAAGAGU | |
| | CAAAUGGCUCUCCUCAAGCGUAUUCAACAAGGGGC | |
| | UGAAGGAUGCCCAGAAGGUACCCCAUUG | |
| 7 | CCCCUCUCCCUCCCCCCCUAACGUUACUGGCCGAAG | Seq 2 |
| | CCGCUUGGAAUAAGGCCGGUGUGCGUUUGUCUAUA | |
| | UGUUAUUUUCCACCAUAUUGCCGUCUUUUGGCAAU | |
| | GUGAGGGCCCGGAAACCUGGCCCUGUCUUCUUGAC | |
| | GAGCAUUCCUAGGGGUCUUUCCCCUCUCGCCAAAG | |
| | GAAUGCAAGGUCUGUUGAAUGUCGUGAAGGAAGCA | |
| | GUUCCUCUGGAAGCUUCUUGAAGACAAACAACGUC | |
| | UGUAGCGACCCUUUGCAGGCAGCGGAACCCCCCACC | |
| | UGGCGACAGGUGCCUCUGCGGCCAAAAGCCACGUG | |
| | UAUAAGAUACACCUGCAAAGGCGGCACAACCCCAG | |
| | UGCCACGUUGUGAGUUGGAUAGUUGUGGAAAGAGU | |
| | CAAAUGGCUCUCCUCAAGCGUAUUCAACAAGGGGC | |
| | UGAAGGAUGCCCAGAAGGUACCCCAUUGUAUGGGA | |
| | UCUGAUCUGGGGCCUCGGUGCACAUGCUUUACAUG | |
| | UGUUUAGUCGAGGUUAAAAAACGUCUAGGCCCCCC | |
| | GAACCACGGGGACGUGGUUUUCCUUUGAAAAACAC | |
| | GAUGAUAAUAUGGCCACAACCAUGAACUCCUUCUC | |
| | CACAAGCGCCUUCGGUCCAGUUGCCUUCUCCCUGG | |
| | GCCUGCUCCUGGUGUUGCCUGCUGCCUUCCCUGCCC | |
| | CAGGGGUGCACGAAUGUCCUGCCUGGCUGUGGCUU | |
| | CUCCUGUCCCUGCUGUCGCUCCCUCUGGGCCUCCCA | |
| | GUCCUGGGCGCCCCACCACGCCUCAUCUGUGACAGC | |
| | CGAGUCCUGGAGAGGUACCUCUUGGAGGCCAAGGA | |
| | GGCCGAGAAUAUCACGACGGGCUGUGCUGAACACU | |
| | GCAGCUUGAAUGAGAAUAUCACUGUCCCAGACACC | |
| | AAAGUUAAUUUCUAUGCCUGGAAGAGGAUGGAGGU | |
| | CGGGCAGCAGGCCGUAGAAGUCUGGCAGGGCCUGG | |
| | CCCUGCUGUCGGAAGCUGUCCUGCGGGGCCAGGCC | |
| | CUGUUGGUCAACUCUUCCCAGCCGUGGGAGCCCCU | |
| | GCAGCUGCAUGUGGAUAAAGCCGUCAGUGGCCUUC | |
| | GCAGCCUCACCACUCUGCUUCGGGCUCUGGGAGCCC | |
| | AGAAGGAAGCCAUCUCCCCUCCAGAUGCGGCCUCA | |
| | GCUGCUCCACUCCGAACAAUCACUGCUGACACUUU | |
| | CCGCAAACUCUUCCGAGUCUACUCCAAUUUCCUCCG | |
| | GGGAAAGCUGAAGCUGUACACAGGGGAGGCUUGCA | |
| | GGACAGGGGACAGAUAG | |
| 8 | UAGACACCAC | Arms-10 |
| 9 | UAGACACCACUACGG | Arms-15 |
| 10 | CCGUCGAUUGUCCACUGGU | Arms-19 |
| 11 | AAAUAAUUGAGCCUUAGAGAAGA | Assembly |
| | | intron-1-F1 |
| 12 | AAUUCUUUAAGUGGAUGCUCUCAAACUCAGGGAAA | Assembly |
| | CCUAAAUCUAGCUAUAGACAAGGCAAUCCUGAGCC | intron-1-F2 |
| | AAGCCGAAGUAGUAAUUAGUAAGUUAAC | |
| 13 | AACAGAUAACUUACAGCUAGUCGGAAGGUGCAGAG | Assembly |
| | ACUCGACGGGAGCUACCCUAACGUCAAGACGAGGG | intron-1-F3 |
| | UAAAGAGAGAGUCCAAUUCUCAAAGCCAAUAGGCA | |
| | GUAGCGAAAGCUGCGGGAGAAUG | |
| 14 | AAAUAAUUGAGCCUUAGAGAAGAAAUUCUUUAAGU | Assemblyin |
| | GGAUGCUCUCAAACUCAGGGAAACCUAAAUCUAGC | tron-2-F1 |
| 15 | UAUAGACAAGGCAAUCCUGAGCCAAGCCGAAGUAG | Assembly |
| | UAAUUAGUAAGUUAAC | intron-2-F2 |
| 16 | AACAGAUAACUUACAGCUAGUCGGAAGGUGCAGAG | Assembly |
| | ACUCGACGGGAGCUACCCUAACGUCAAGACGAGGG | intron-2-F3 |
| | UAAAGAGAGAGUCCAAUUCUCAAAGCCAAUAGGCA | |
| | GUAGCGAAAGCUGCGGGAGAAUG | |
| 17 | AAAUAAUUGAGCCUUAGAGAAGAAAUUCUUUAAGU | Assembly |
| | GGAUGCUCUCAAACUCAGGGAAACCUAAAUCUAGC | intron-3-F1 |
| | UAUAGACAAGGCAAUCCUGAGCCAAGCCGAAGUAG | |
| | UAAUUAGUAAGUUAAC | |
| 18 | AACAGAUAACUUACAGCUAGUCGGAAGGUGCAGAG | Assembly |
| | ACUCGACGGGAGCUACCCUAACGUCA | intron-3-F2 |
| 19 | AGACGAGGGUAAAGAGAGAGUCCAAUUCUCAAAGC | Assembly |
| | CAAUAGGCAGUAGCGAAAGCUGCGGGAGAAUG | intron-3-F3 |
| 20 | AAAUAAUUGAGCCUUAGAGAAGAAAUUCUUUAAGU | Assembly |
| | GGAUGCUCUCAAACUCAGGGAAACCUAAAUCUAGC | intron-4-F1 |
| | UAUAGACAAGGCAAUCCUGAGCCAAGCCGAAGUAG | |
| | UAAUUAGUAAGUUAAC | |
| 21 | AACAGAUAACUUACAGCUAGUCGGAAGGUGCAGAG | Assembly |
| | ACUCGACGGGAGCUACCCUAACGUCAAGACGAGGG | intron-4-F2 |
| | UAAAGAGAGAGUCCAAUUCUCAAAGCCAA | |
| 22 | UAGGCAGUAGCGAAAGCUGCGGGAGAAUG | Assembly |
| | | intron-4-F3 |
| 23 | UAUAGACAAGGCAAUCCUGAGCCAAG | Assembly |
| | | intron-2-1- |
| | | F2 |
| 24 | CCGAAGUAGUAAUUAGUAAGUUAACAACAGAUAAC | Assembly |
| | UUACAGCUAGUCGGAAGGUGCAGAGACUCGACGGG | intron-2-1- |
| | AGCUACCCUAACGUCAAGACGAGGGUAAAGAGAGA | F3 |
| | GUCCAAUUCUCAAAGCCAAUAGGCAGUAGCGAAAG | |
| | CUGCGGGAGAAUG | |
| 25 | UAUAGACAAGGCAAUCCUGAGCCAAGCCGAAGCCG | Assembly |
| | | intron-2-2- |
| | | F2 |
| 26 | UAGUAAUUAGUAAGUUAACAACAGAUAACUUACAG | Assembly |
| | CUAGUCGGAAGGUGCAGAGACUCGACGGGAGCUAC | intron-2-2- |
| | CCUAACGUCAAGACGAGGGUAAAGAGAGAGUCCAA | F3 |
| | UUCUCAAAGCCAAUAGGCAGUAGCGAAAGCUGCGG | |
| | GAGAAUG | |
| 27 | UAUAGACAAGGCAAUCCUGAGCCAAGCCGAAGUAG | Assembly |
| | UAA | intron-2-3- |
| | | F2 |
| 28 | UUAGUAAGUUAACAACAGAUAACUUACAGCUAGUC | Assembly |
| | GGAAGGUGCAGAGACUCGACGGGAGCUACCCUAAC | intron-2-3- |
| | GUCAAGACGAGGGUAAAGAGAGAGUCCAAUUCUCA | F3 |
| | AAGCCAAUAGGCAGUAGCGAAAGCUGCGGGAGAAU | |
| | G | |
| 29 | UAUAGACAAGGCAAUCCUGAGCCAAGCCGAAGUAG | Assembly |
| | UAAUUA | intron-2-4- |
| | | F2 |
| 30 | GUAAGUUAACAACAGAUAACUUACAGCUAGUCGGA | Assembly |
| | AGGUGCAGAGACUCGACGGGAGCUACCCUAACGUC | intron-2-4- |
| | AAGACGAGGGUAAAGAGAGAGUCCAAUUCUCAAAG | F3 |
| | CCAAUAGGCAGUAGCGAAAGCUGCGGGAGAAUG | |
| 31 | AACAGAUAACUUACAGCUAGUCGGAAGGUGCAGAG | P-AI-1 |
| | ACUCGACGGGAGCUACCCUAACGUCAAGACGAGGG | |
| | UAAAGAGAGAGUCCAAUUCUCAAAGCCAAUAGGCA | |
| | GUAGCGAAAGCUGCGGGAGAAUGAAAAUCCGUUGA | |
| | CCUUAAACGGUCGUGUGGGUUCAAGUCCCUCCACC | |
| | CCCAuggcggaaugguAGACGCUACGGACUUAAAUAAUU | |
| | GAGCCUUAGAGAAGA | |
| 32 | AACAGAUAACUUACAGCUAGUCGGAAGGUGCAGAG | P-AI-2 |
| | ACUCGACGGGAGCUACCCUAACGUCAAGACGAGGG | |
| | UAAAGAGAGAGUCCAAUUCUCAAAGCCAAUAGGCA | |
| | GUAGCGAAAGCUGCGGGAGAAUGAAAAUCCGUUGA | |
| | CCUUAAACGGUCGUGUGGGUUCAAGUCCCUCCACC | |
| | CCCAuggcggaaugguAGACGCUACGGACUUAAAUAAUU | |
| | GAGCCUUAGAGAAGAAAUUCUUUAAGUGGAUGCUC | |
| | UCAAACUCAGGGAAACCUAAAUCUAGC | |
| 33 | AGACGAGGGUAAAGAGAGAGUCCAAUUCUCAAAGC | P-AI-3 |
| | CAAUAGGCAGUAGCGAAAGCUGCGGGAGAAUGAAA | |
| | AUCCGUUGACCUUAAACGGUCGUGUGGGUUCAAGU | |
| | CCCUCCACCCCCAuggcggaaugguAGACGCUACGGACU | |
| | UAAAUAAUUGAGCCUUAGAGAAGAAAUUCUUUAAG | |
| | UGGAUGCUCUCAAACUCAGGGAAACCUAAAUCUAG | |
| | CUAUAGACAAGGCAAUCCUGAGCCAAGCCGAAGUA | |
| | GUAAUUAGUAAGUUAAC | |
| 34 | UAGGCAGUAGCGAAAGCUGCGGGAGAAUGAAAAUC | P-AI-4 |
| | CGUUGACCUUAAACGGUCGUGUGGGUUCAAGUCCC | |
| | UCCACCCCCAuggcggaaugguAGACGCUACGGACUUAA | |
| | AUAAUUGAGCCUUAGAGAAGAAAUUCUUUAAGUGG | |
| | AUGCUCUCAAACUCAGGGAAACCUAAAUCUAGCUA | |
| | UAGACAAGGCAAUCCUGAGCCAAGCCGAAGUAGUA | |
| | AUUAGUAAGUUAAC | |
| 35 | CCGAAGUAGUAAUUAGUAAGUUAACAACAGAUAAC | P-AI-2-1 |
| | UUACAGCUAGUCGGAAGGUGCAGAGACUCGACGGG | |
| | AGCUACCCUAACGUCAAGACGAGGGUAAAGAGAGA | |
| | GUCCAAUUCUCAAAGCCAAUAGGCAGUAGCGAAAG | |
| | CUGCGGGAGAAUGAAAAUCCGUUGACCUUAAACGG | |
| | UCGUGUGGGUUCAAGUCCCUCCACCCCCAuggcggaaug | |
| | guAGACGCUACGGACUUAAAUAAUUGAGCCUUAGA | |
| | GAAGAAAUUCUUUAAGUGGAUGCUCUCAAACUCAG | |
| | GGAAACCUAAAUCUAGC | |
| 36 | UAGUAAUUAGUAAGUUAACAACAGAUAACUUACAG | P-AI-2-2 |
| | CUAGUCGGAAGGUGCAGAGACUCGACGGGAGCUAC | |
| | CCUAACGUCAAGACGAGGGUAAAGAGAGAGUCCAA | |
| | UUCUCAAAGCCAAUAGGCAGUAGCGAAAGCUGCGG | |
| | GAGAAUGAAAAUCCGUUGACCUUAAACGGUCGUGU | |
| | GGGUUCAAGUCCCUCCACCCCCAuggcggaaugguAGAC | |
| | GCUACGGACUUAAAUAAUUGAGCCUUAGAGAAGAA | |
| | AUUCUUUAAGUGGAUGCUCUCAAACUCAGGGAAAC | |
| | CUAAAUCUAGC | |
| 37 | UUAGUAAGUUAACAACAGAUAACUUACAGCUAGUC | P-AI-2-3 |
| | GGAAGGUGCAGAGACUCGACGGGAGCUACCCUAAC | |
| | GUCAAGACGAGGGUAAAGAGAGAGUCCAAUUCUCA | |
| | AAGCCAAUAGGCAGUAGCGAAAGCUGCGGGAGAAU | |
| | GAAAAUCCGUUGACCUUAAACGGUCGUGUGGGUUC | |
| | AAGUCCCUCCACCCCCAuggcggaaugguAGACGCUACG | |
| | GACUUAAAUAAUUGAGCCUUAGAGAAGAAAUUCUU | |
| | UAAGUGGAUGCUCUCAAACUCAGGGAAACCUAAAU | |
| | CUAGC | |
| 38 | GUAAGUUAACAACAGAUAACUUACAGCUAGUCGGA | P-AI-2-4 |
| | AGGUGCAGAGACUCGACGGGAGCUACCCUAACGUC | |
| | AAGACGAGGGUAAAGAGAGAGUCCAAUUCUCAAAG | |
| | CCAAUAGGCAGUAGCGAAAGCUGCGGGAGAAUGAA | |
| | AAUCCGUUGACCUUAAACGGUCGUGUGGGUUCAAG | |
| | UCCCUCCACCCCCAuggcggaaugguAGACGCUACGGAC | |
| | UUAAAUAAUUGAGCCUUAGAGAAGAAAUUCUUUAA | |
| | GUGGAUGCUCUCAAACUCAGGGAAACCUAAAUCUA | |
| | GC | |
| 39 | AAAAUCCGUUGACCUUAAACGGUCGUGUGGGUUCA | circ-Exon |
| | AGUCCCUCCACCCCCAUGGCGGAAUGGUAGACGCU | |
| | ACGGACUU | |
| 40 | AACAGAUAACUUACAGCUAGUCGGAAGGUGCAGAG | P-RNA-seq |
| | ACUCGACGGGAGCUACCCUAACGUCAAGACGAGGG | 1 |
| | UAAAGAGAGAGUCCAAUUCUCAAAGCCAAUAGGCA | |
| | GUAGCGAAAGCUGCGGGAGAAUG | |
| | AAAAUCCGUUGACCUUAAACGGUCGUGUGGGUUCA | |
| | AGUCCCUCCACCCCCA | |
| | CCCCUCUCCCUCCCCCCCUAACGUUACUGGCCGAAG | |
| | CCGCUUGGAAUAAGGCCGGUGUGCGUUUGUCUAUA | |
| | UGUUAUUUUCCACCAUAUUGCCGUCUUUUGGCAAU | |
| | GUGAGGGCCCGGAAACCUGGCCCUGUCUUCUUGAC | |
| | GAGCAUUCCUAGGGGUCUUUCCCCUCUCGCCAAAG | |
| | GAAUGCAAGGUCUGUUGAAUGUCGUGAAGGAAGCA | |
| | GUUCCUCUGGAAGCUUCUUGAAGACAAACAACGUC | |
| | UGUAGCGACCCUUUGCAGGCAGCGGAACCCCCCACC | |
| | UGGCGACAGGUGCCUCUGCGGCCAAAAGCCACGUG | |
| | UAUAAGAUACACCUGCAAAGGCGGCACAACCCCAG | |
| | UGCCACGUUGUGAGUUGGAUAGUUGUGGAAAGAGU | |
| | CAAAUGGCUCUCCUCAAGCGUAUUCAACAAGGGGC | |
| | UGAAGGAUGCCCAGAAGGUACCCCAUUG | |
| | UGGCGGAAUGGUAGACGCUACGGACUU | |
| | AAAUAAUUGAGCCUUAGAGAAGAAAUUCUUUAAGU | |
| | GGAUGCUCUCAAACUCAGGGAAACCUAAAUCUAGC | |
| 41 | GUGGUUAUAGACAAGGCAAUCCUGAGCCAAGCCGA | BM(5)-F2 |
| | AGUAGUAA | |
| 42 | GUGGUGUCUAUAUAGACAAGGCAAUCCUGAGCCAA | BM(10)-F2 |
| | GCCGAAGUAGUAAUUAGUAAGUUA | |
| 43 | CCGUAGUGGUGUCUAUAUAGACAAGGCAAUCCUGA | BM(15)-F2 |
| | GCCAAGCCGAAGUAGUAAUUAGUAAGUUA | |
| 44 | GAGTACCGUAGUGGUGUCUAUAUAGACAAGGCAAU | BM(20)-F2 |
| | CCUGAGCCAAGCCGAAGUAGUAAUUAGUAAGUUA | |
| 45 | UGGCAGCACAGGAUGAUGGCACACUGCCGAGAGUA | BM(50)-F2 |
| | CCGUAGUGGUGUCUAUAUAGACAAGGCAAUCCUGA | |
| | GCCAAGCCGAAGUAGUAAUUAGUAAGUUA | |
| 46 | AACAGAUAACUUACAGCUAGUCGGAAGGUGCAGAG | I(5)-P- |
| | ACUCGACGGGAGCUACCCUAACGUCAAGACGAGGG | RNA-seq 1 |
| | UAAAGAGAGAGUCCAAUUCUCAAAGCCAAUAGGCA | |
| | GUAGCGAAAGCUGCGGGAGAAUG | |
| | AAAAUCCGUUGACCUUAAACGGUCGUGUGGGUUCA | |
| | AGUCCCUCCACCCCCA | |
| | CCCCUCUCCCUCCCCCCCUAACGUUACUGGCCGAAG | |
| | CCGCUUGGAAUAAGGCCGGUGUGCGUUUGUCUAUA | |
| | UGUUAUUUUCCACCAUAUUGCCGUCUUUUGGCAAU | |
| | GUGAGGGCCCGGAAACCUGGCCCUGUCUUCUUGAC | |
| | GAGCAUUCCUAGGGGUCUUUCCCCUCUCGCCAAAG | |
| | GAAUGCAAGGUCUGUUGAAUGUCGUGAAGGAAGCA | |
| | GUUCCUCUGGAAGCUUCUUGAAGACAAACAACGUC | |
| | UGUAGCGACCCUUUGCAGGCAGCGGAACCCCCCACC | |
| | UGGCGACAGGUGCCUCUGCGGCCAAAAGCCACGUG | |
| | UAUAAGAUACACCUGCAAAGGCGGCACAACCCCAG | |
| | UGCCACGUUGUGAGUUGGAUAGUUGUGGAAAGAGU | |
| | CAAAUGGCUCUCCUCAAGCGUAUUCAACAAGGGGC | |
| | UGAAGGAUGCCCAGAAGGUACCCCAUUG | |
| | uggcggaaugguAGACGCUACGGACUU | |
| | AAAUAAUUGAGCCUUAGAGAAGAAAUUCUUUAAGU | |
| | GGAUGCUCUCAAACUCAGGGAAACCUAAAUCUAGC | |
| | ACCAC | |
| 47 | AACAGAUAACUUACAGCUAGUCGGAAGGUGCAGAG | I(10)-P- |
| | ACUCGACGGGAGCUACCCUAACGUCAAGACGAGGG | RNA-seq 1 |
| | UAAAGAGAGAGUCCAAUUCUCAAAGCCAAUAGGCA | |
| | GUAGCGAAAGCUGCGGGAGAAUG | |
| | AAAAUCCGUUGACCUUAAACGGUCGUGUGGGUUCA | |
| | AGUCCCUCCACCCCCA | |
| | CCCCUCUCCCUCCCCCCCUAACGUUACUGGCCGAAG | |
| | CCGCUUGGAAUAAGGCCGGUGUGCGUUUGUCUAUA | |
| | UGUUAUUUUCCACCAUAUUGCCGUCUUUUGGCAAU | |
| | GUGAGGGCCCGGAAACCUGGCCCUGUCUUCUUGAC | |
| | GAGCAUUCCUAGGGGUCUUUCCCCUCUCGCCAAAG | |
| | GAAUGCAAGGUCUGUUGAAUGUCGUGAAGGAAGCA | |
| | GUUCCUCUGGAAGCUUCUUGAAGACAAACAACGUC | |
| | UGUAGCGACCCUUUGCAGGCAGCGGAACCCCCCACC | |
| | UGGCGACAGGUGCCUCUGCGGCCAAAAGCCACGUG | |
| | UAUAAGAUACACCUGCAAAGGCGGCACAACCCCAG | |
| | UGCCACGUUGUGAGUUGGAUAGUUGUGGAAAGAGU | |
| | CAAAUGGCUCUCCUCAAGCGUAUUCAACAAGGGGC | |
| | UGAAGGAUGCCCAGAAGGUACCCCAUUG | |
| | uggcggaaugguAGACGCUACGGACUU | |
| | AAAUAAUUGAGCCUUAGAGAAGAAAUUCUUUAAGU | |
| | GGAUGCUCUCAAACUCAGGGAAACCUAAAUCUAGC | |
| | UAGACACCAC | |
| 48 | AACAGAUAACUUACAGCUAGUCGGAAGGUGCAGAG | I(15)-P- |
| | ACUCGACGGGAGCUACCCUAACGUCAAGACGAGGG | RNA-seq 1 |
| | UAAAGAGAGAGUCCAAUUCUCAAAGCCAAUAGGCA | |
| | GUAGCGAAAGCUGCGGGAGAAUG | |
| | AAAAUCCGUUGACCUUAAACGGUCGUGUGGGUUCA | |
| | AGUCCCUCCACCCCCA | |
| | CCCCUCUCCCUCCCCCCCUAACGUUACUGGCCGAAG | |
| | CCGCUUGGAAUAAGGCCGGUGUGCGUUUGUCUAUA | |
| | UGUUAUUUUCCACCAUAUUGCCGUCUUUUGGCAAU | |
| | GUGAGGGCCCGGAAACCUGGCCCUGUCUUCUUGAC | |
| | GAGCAUUCCUAGGGGUCUUUCCCCUCUCGCCAAAG | |
| | GAAUGCAAGGUCUGUUGAAUGUCGUGAAGGAAGCA | |
| | GUUCCUCUGGAAGCUUCUUGAAGACAAACAACGUC | |
| | UGUAGCGACCCUUUGCAGGCAGCGGAACCCCCCACC | |
| | UGGCGACAGGUGCCUCUGCGGCCAAAAGCCACGUG | |
| | UAUAAGAUACACCUGCAAAGGCGGCACAACCCCAG | |
| | UGCCACGUUGUGAGUUGGAUAGUUGUGGAAAGAGU | |
| | CAAAUGGCUCUCCUCAAGCGUAUUCAACAAGGGGC | |
| | UGAAGGAUGCCCAGAAGGUACCCCAUUG | |
| | uggcggaaugguAGACGCUACGGACUU | |
| | AAAUAAUUGAGCCUUAGAGAAGAAAUUCUUUAAGU | |
| | GGAUGCUCUCAAACUCAGGGAAACCUAAAUCUAGC | |
| | UAGACACCACUACGG | |
| 49 | AACAGAUAACUUACAGCUAGUCGGAAGGUGCAGAG | I(20)-P- |
| | ACUCGACGGGAGCUACCCUAACGUCAAGACGAGGG | RNA-seq 1 |
| | UAAAGAGAGAGUCCAAUUCUCAAAGCCAAUAGGCA | |
| | GUAGCGAAAGCUGCGGGAGAAUG | |
| | AAAAUCCGUUGACCUUAAACGGUCGUGUGGGUUCA | |
| | AGUCCCUCCACCCCCA | |
| | CCCCUCUCCCUCCCCCCCUAACGUUACUGGCCGAAG | |
| | CCGCUUGGAAUAAGGCCGGUGUGCGUUUGUCUAUA | |
| | UGUUAUUUUCCACCAUAUUGCCGUCUUUUGGCAAU | |
| | GUGAGGGCCCGGAAACCUGGCCCUGUCUUCUUGAC | |
| | GAGCAUUCCUAGGGGUCUUUCCCCUCUCGCCAAAG | |
| | GAAUGCAAGGUCUGUUGAAUGUCGUGAAGGAAGCA | |
| | GUUCCUCUGGAAGCUUCUUGAAGACAAACAACGUC | |
| | UGUAGCGACCCUUUGCAGGCAGCGGAACCCCCCACC | |
| | UGGCGACAGGUGCCUCUGCGGCCAAAAGCCACGUG | |
| | UAUAAGAUACACCUGCAAAGGCGGCACAACCCCAG | |
| | UGCCACGUUGUGAGUUGGAUAGUUGUGGAAAGAGU | |
| | CAAAUGGCUCUCCUCAAGCGUAUUCAACAAGGGGC | |
| | UGAAGGAUGCCCAGAAGGUACCCCAUUG | |
| | uggcggaaugguAGACGCUACGGACUU | |
| | AAAUAAUUGAGCCUUAGAGAAGAAAUUCUUUAAGU | |
| | GGAUGCUCUCAAACUCAGGGAAACCUAAAUCUAGC | |
| | UAGACACCACUACGGUACUC | |
| 50 | AACAGAUAACUUACAGCUAGUCGGAAGGUGCAGAG | I(50)-P- |
| | ACUCGACGGGAGCUACCCUAACGUCAAGACGAGGG | RNA-seq 1 |
| | UAAAGAGAGAGUCCAAUUCUCAAAGCCAAUAGGCA | |
| | GUAGCGAAAGCUGCGGGAGAAUG | |
| | AAAAUCCGUUGACCUUAAACGGUCGUGUGGGUUCA | |
| | AGUCCCUCCACCCCCA | |
| | CCCCUCUCCCUCCCCCCCUAACGUUACUGGCCGAAG | |
| | CCGCUUGGAAUAAGGCCGGUGUGCGUUUGUCUAUA | |
| | UGUUAUUUUCCACCAUAUUGCCGUCUUUUGGCAAU | |
| | GUGAGGGCCCGGAAACCUGGCCCUGUCUUCUUGAC | |
| | GAGCAUUCCUAGGGGUCUUUCCCCUCUCGCCAAAG | |
| | GAAUGCAAGGUCUGUUGAAUGUCGUGAAGGAAGCA | |
| | GUUCCUCUGGAAGCUUCUUGAAGACAAACAACGUC | |
| | UGUAGCGACCCUUUGCAGGCAGCGGAACCCCCCACC | |
| | UGGCGACAGGUGCCUCUGCGGCCAAAAGCCACGUG | |
| | UAUAAGAUACACCUGCAAAGGCGGCACAACCCCAG | |
| | UGCCACGUUGUGAGUUGGAUAGUUGUGGAAAGAGU | |
| | CAAAUGGCUCUCCUCAAGCGUAUUCAACAAGGGGC | |
| | UGAAGGAUGCCCAGAAGGUACCCCAUUG | |
| | uggcggaaugguAGACGCUACGGACUU | |
| | AAAUAAUUGAGCCUUAGAGAAGAAAUUCUUUAAGU | |
| | GGAUGCUCUCAAACUCAGGGAAACCUAAAUCUAGC | |
| | UAGACACCACUACGGUACUCUCGGCAGUGUGCCAU | |
| | CAUCCUGUGCUGCCA | |
| 51 | CCCCUCUCCCUCCCCCCCUAACGUUACUGGCCGAAG | EMCV |
| | CCGCUUGGAAUAAGGCCGGUGUGCGUUUGUCUAUA | IRES |
| | UGUUAUUUUCCACCAUAUUGCCGUCUUUUGGCAAU | |
| | GUGAGGGCCCGGAAACCUGGCCCUGUCUUCUUGAC | |
| | GAGCAUUCCUAGGGGUCUUUCCCCUCUCGCCAAAG | |
| | GAAUGCAAGGUCUGUUGAAUGUCGUGAAGGAAGCA | |
| | GUUCCUCUGGAAGCUUCUUGAAGACAAACAACGUC | |
| | UGUAGCGACCCUUUGCAGGCAGCGGAACCCCCCACC | |
| | UGGCGACAGGUGCCUCUGCGGCCAAAAGCCACGUG | |
| | UAUAAGAUACACCUGCAAAGGCGGCACAACCCCAG | |
| | UGCCACGUUGUGAGUUGGAUAGUUGUGGAAAGAGU | |
| | CAAAUGGCUCUCCUCAAGCGUAUUCAACAAGGGGC | |
| | UGAAGGAUGCCCAGAAGGUACCCCAUUGUAUGGGA | |
| | UCUGAUCUGGGGCCUCGGUGCACAUGCUUUACAUG | |
| | UGUUUAGUCGAGGUUAAAAAACGUCUAGGCCCCCC | |
| | GAACCACGGGGACGUGGUUUUCCUUUGAAAAACAC | |
| | GAUGAUAAUAUGGCCACAACC | |
| 52 | AUGGGAGUCAAAGUUCUGUUUGCCCUGAUCUGCAU | Gaussia |
| | CGCUGUGGCCGAGGCCAAGCCCACCGAGAACAACG | luciferase |
| | AAGACUUCAACAUCGUGGCCGUGGCCAGCAACUUC | |
| | GCGACCACGGAUCUCGAUGCUGACCGCGGGAAGUU | |
| | GCCCGGCAAGAAGCUGCCGCUGGAGGUGCUCAAAG | |
| | AGAUGGAAGCCAAUGCCCGGAAAGCUGGCUGCACC | |
| | AGGGGCUGUCUGAUCUGCCUGUCCCACAUCAAGUG | |
| | CACGCCCAAGAUGAAGAAGUUCAUCCCAGGACGCU | |
| | GCCACACCUACGAAGGCGACAAAGAGUCCGCACAG | |
| | GGCGGCAUAGGCGAGGCGAUCGUCGACAUUCCUGA | |
| | GAUUCCUGGGUUCAAGGACUUGGAGCCCAUGGAGC | |
| | AGUUCAUCGCACAGGUCGAUCUGUGUGUGGACUGC | |
| | ACAACUGGCUGCCUCAAAGGGCUUGCCAACGUGCA | |
| | GUGUUCUGACCUGCUCAAGAAGUGGCUGCCGCAAC | |
| | GCUGUGCGACCUUUGCCAGCAAGAUCCAGGGCCAG | |
| | GUGGACAAGAUCAAGGGGGCCGGUGGUGACUAA | |
| 53 | AACAGAUAACUUACAGCUAGUCGGAAGGUGCAGAG | I-P-RNA- |
| | ACUCGACGGGAGCUACCCUAACGUCAAGACGAGGG | Gluc |
| | UAAAGAGAGAGUCCAAUUCUCAAAGCCAAUAGGCA | |
| | GUAGCGAAAGCUGCGGGAGAAUG | |
| | AAAAUCCGUUGACCUUAAACGGUCGUGUGGGUUCA | |
| | AGUCCCUCCACCCCCA | |
| | CCCCUCUCCCUCCCCCCCUAACGUUACUGGCCGAAG | |
| | CCGCUUGGAAUAAGGCCGGUGUGCGUUUGUCUAUA | |
| | UGUUAUUUUCCACCAUAUUGCCGUCUUUUGGCAAU | |
| | GUGAGGGCCCGGAAACCUGGCCCUGUCUUCUUGAC | |
| | GAGCAUUCCUAGGGGUCUUUCCCCUCUCGCCAAAG | |
| | GAAUGCAAGGUCUGUUGAAUGUCGUGAAGGAAGCA | |
| | GUUCCUCUGGAAGCUUCUUGAAGACAAACAACGUC | |
| | UGUAGCGACCCUUUGCAGGCAGCGGAACCCCCCACC | |
| | UGGCGACAGGUGCCUCUGCGGCCAAAAGCCACGUG | |
| | UAUAAGAUACACCUGCAAAGGCGGCACAACCCCAG | |
| | UGCCACGUUGUGAGUUGGAUAGUUGUGGAAAGAGU | |
| | CAAAUGGCUCUCCUCAAGCGUAUUCAACAAGGGGC | |
| | UGAAGGAUGCCCAGAAGGUACCCCAUUGUAUGGGA | |
| | UCUGAUCUGGGGCCUCGGUGCACAUGCUUUACAUG | |
| | UGUUUAGUCGAGGUUAAAAAACGUCUAGGCCCCCC | |
| | GAACCACGGGGACGUGGUUUUCCUUUGAAAAACAC | |
| | GAUGAUAAUAUGGCCACAACC | |
| | AUGGGAGUCAAAGUUCUGUUUGCCCUGAUCUGCAU | |
| | CGCUGUGGCCGAGGCCAAGCCCACCGAGAACAACG | |
| | AAGACUUCAACAUCGUGGCCGUGGCCAGCAACUUC | |
| | GCGACCACGGAUCUCGAUGCUGACCGCGGGAAGUU | |
| | GCCCGGCAAGAAGCUGCCGCUGGAGGUGCUCAAAG | |
| | AGAUGGAAGCCAAUGCCCGGAAAGCUGGCUGCACC | |
| | AGGGGCUGUCUGAUCUGCCUGUCCCACAUCAAGUG | |
| | CACGCCCAAGAUGAAGAAGUUCAUCCCAGGACGCU | |
| | GCCACACCUACGAAGGCGACAAAGAGUCCGCACAG | |
| | GGCGGCAUAGGCGAGGCGAUCGUCGACAUUCCUGA | |
| | GAUUCCUGGGUUCAAGGACUUGGAGCCCAUGGAGC | |
| | AGUUCAUCGCACAGGUCGAUCUGUGUGUGGACUGC | |
| | ACAACUGGCUGCCUCAAAGGGCUUGCCAACGUGCA | |
| | GUGUUCUGACCUGCUCAAGAAGUGGCUGCCGCAAC | |
| | GCUGUGCGACCUUUGCCAGCAAGAUCCAGGGCCAG | |
| | GUGGACAAGAUCAAGGGGGCCGGUGGUGACUAA | |
| | uggcggaaugguAGACGCUACGGACUU | |
| | AAAUAAUUGAGCCUUAGAGAAGAAAUUCUUUAAGU | |
| | GGAUGCUCUCAAACUCAGGGAAACCUAAAUCUAGC | |
| | UAGACACCAC | |
| 54 | AUGGAGGACGCCAAGAACAUUAAGAAGGGCCCAGC | Firefly |
| | CCCAUUCUACCCCCUGGAAGACGGCACUGCCGGCGA | luciferase |
| | GCAGCUGCACAAGGCCAUGAAACGGUAUGCACUGG | |
| | UGCCCGGCACAAUCGCCUUCACCGACGCUCAUAUCG | |
| | AGGUGGAUAUCACUUACGCCGAGUACUUCGAGAUG | |
| | AGCGUCCGGCUGGCUGAGGCCAUGAAGAGAUAUGG | |
| | CCUGAACACCAAUCAUCGGAUCGUGGUGUGCAGCG | |
| | AAAACAGCCUGCAGUUUUUCAUGCCCGUCCUGGGC | |
| | GCCCUGUUCAUCGGAGUGGCCGUGGCCCCCGCCAA | |
| | UGAUAUUUACAAUGAACGGGAACUGCUGAACAGCA | |
| | UGGGCAUUAGCCAGCCAACUGUGGUGUUCGUGAGC | |
| | AAAAAGGGCCUGCAGAAGAUCCUGAACGUGCAGAA | |
| | GAAACUGCCCAUUAUCCAGAAGAUCAUCAUCAUGG | |
| | ACAGCAAGACCGAUUAUCAGGGGUUCCAGAGCAUG | |
| | UAUACCUUCGUGACAAGCCAUCUGCCCCCCGGCUU | |
| | UAAUGAGUAUGAUUUUGUGCCCGAAAGCUUCGAUA | |
| | GGGAUAAAACCAUCGCCCUGAUCAUGAACAGCAGC | |
| | GGCUCCACCGGGCUGCCCAAAGGCGUGGCACUGCCC | |
| | CAUCGGACAGCCUGCGUGCGGUUCUCCCACGCUCG | |
| | GGACCCCAUCUUCGGGAACCAGAUCAUCCCCGACAC | |
| | CGCCAUUCUGAGCGUGGUGCCCUUUCAUCAUGGCU | |
| | UUGGCAUGUUUACCACCCUGGGCUACCUCAUCUGU | |
| | GGCUUCAGGGUGGUGCUCAUGUAUCGCUUUGAGGA | |
| | AGAACUGUUUCUGCGGAGCCUGCAGGACUACAAGA | |
| | UCCAGAGCGCUCUGCUGGUGCCCACCCUGUUUAGC | |
| | UUCUUCGCCAAGAGCACCCUCAUUGAUAAGUAUGA | |
| | UCUGAGCAAUCUGCACGAGAUCGCCAGCGGCGGGG | |
| | CCCCCCUGAGCAAGGAGGUGGGAGAAGCCGUGGCC | |
| | AAACGGUUCCACCUCCCUGGGAUCAGGCAGGGCUA | |
| | UGGACUGACCGAAACCACCAGCGCCAUCCUGAUCA | |
| | CCCCCGAAGGCGAUGACAAGCCCGGCGCUGUGGGC | |
| | AAAGUGGUGCCCUUCUUCGAGGCUAAGGUGGUGGA | |
| | UCUGGACACUGGCAAAACCCUGGGGGUGAACCAGC | |
| | GGGGCGAGCUGUGCGUGCGGGGGCCUAUGAUUAUG | |
| | AGCGGCUACGUGAAUAACCCCGAGGCAACCAAUGC | |
| | CCUGAUCGACAAAGAUGGCUGGCUGCACUCUGGGG | |
| | ACAUUGCCUAUUGGGACGAAGACGAGCACUUCUUC | |
| | AUCGUCGAUCGGCUGAAGAGCCUGAUCAAGUAUAA | |
| | AGGCUACCAGGUGGCCCCCGCCGAACUGGAGAGCA | |
| | UUCUGCUGCAGCACCCCAACAUCUUUGAUGCCGGC | |
| | GUGGCAGGCCUGCCAGAUGAUGACGCCGGCGAACU | |
| | GCCCGCCGCCGUGGUGGUGCUGGAGCACGGCAAGA | |
| | CCAUGACCGAGAAAGAAAUCGUGGACUAUGUGGCC | |
| | UCUCAGGUGACCACCGCCAAGAAGCUGCGGGGCGG | |
| | GGUGGUGUUCGUCGACGAGGUCCCAAAGGGCCUGA | |
| | CCGGCAAGCUGGAUGCCCGGAAAAUCCGGGAGAUC | |
| | CUGAUCAAGGCCAAGAAAGGCGGCAAGAUCGCCGU | |
| | Guaa | |
| 55 | AACAGAUAACUUACAGCUAGUCGGAAGGUGCAGAG | I-P-RNA- |
| | ACUCGACGGGAGCUACCCUAACGUCAAGACGAGGG | Fluc |
| | UAAAGAGAGAGUCCAAUUCUCAAAGCCAAUAGGCA | |
| | GUAGCGAAAGCUGCGGGAGAAUG | |
| | AAAAUCCGUUGACCUUAAACGGUCGUGUGGGUUCA | |
| | AGUCCCUCCACCCCCA | |
| | CCCCUCUCCCUCCCCCCCUAACGUUACUGGCCGAAG | |
| | CCGCUUGGAAUAAGGCCGGUGUGCGUUUGUCUAUA | |
| | UGUUAUUUUCCACCAUAUUGCCGUCUUUUGGCAAU | |
| | GUGAGGGCCCGGAAACCUGGCCCUGUCUUCUUGAC | |
| | GAGCAUUCCUAGGGGUCUUUCCCCUCUCGCCAAAG | |
| | GAAUGCAAGGUCUGUUGAAUGUCGUGAAGGAAGCA | |
| | GUUCCUCUGGAAGCUUCUUGAAGACAAACAACGUC | |
| | UGUAGCGACCCUUUGCAGGCAGCGGAACCCCCCACC | |
| | UGGCGACAGGUGCCUCUGCGGCCAAAAGCCACGUG | |
| | UAUAAGAUACACCUGCAAAGGCGGCACAACCCCAG | |
| | UGCCACGUUGUGAGUUGGAUAGUUGUGGAAAGAGU | |
| | CAAAUGGCUCUCCUCAAGCGUAUUCAACAAGGGGC | |
| | UGAAGGAUGCCCAGAAGGUACCCCAUUGUAUGGGA | |
| | UCUGAUCUGGGGCCUCGGUGCACAUGCUUUACAUG | |
| | UGUUUAGUCGAGGUUAAAAAACGUCUAGGCCCCCC | |
| | GAACCACGGGGACGUGGUUUUCCUUUGAAAAACAC | |
| | GAUGAUAAUAUGGCCACAACC | |
| | AUGGAGGACGCCAAGAACAUUAAGAAGGGCCCAGC | |
| | CCCAUUCUACCCCCUGGAAGACGGCACUGCCGGCGA | |
| | GCAGCUGCACAAGGCCAUGAAACGGUAUGCACUGG | |
| | UGCCCGGCACAAUCGCCUUCACCGACGCUCAUAUCG | |
| | AGGUGGAUAUCACUUACGCCGAGUACUUCGAGAUG | |
| | AGCGUCCGGCUGGCUGAGGCCAUGAAGAGAUAUGG | |
| | CCUGAACACCAAUCAUCGGAUCGUGGUGUGCAGCG | |
| | AAAACAGCCUGCAGUUUUUCAUGCCCGUCCUGGGC | |
| | GCCCUGUUCAUCGGAGUGGCCGUGGCCCCCGCCAA | |
| | UGAUAUUUACAAUGAACGGGAACUGCUGAACAGCA | |
| | UGGGCAUUAGCCAGCCAACUGUGGUGUUCGUGAGC | |
| | AAAAAGGGCCUGCAGAAGAUCCUGAACGUGCAGAA | |
| | GAAACUGCCCAUUAUCCAGAAGAUCAUCAUCAUGG | |
| | ACAGCAAGACCGAUUAUCAGGGGUUCCAGAGCAUG | |
| | UAUACCUUCGUGACAAGCCAUCUGCCCCCCGGCUU | |
| | UAAUGAGUAUGAUUUUGUGCCCGAAAGCUUCGAUA | |
| | GGGAUAAAACCAUCGCCCUGAUCAUGAACAGCAGC | |
| | GGCUCCACCGGGCUGCCCAAAGGCGUGGCACUGCCC | |
| | CAUCGGACAGCCUGCGUGCGGUUCUCCCACGCUCG | |
| | GGACCCCAUCUUCGGGAACCAGAUCAUCCCCGACAC | |
| | CGCCAUUCUGAGCGUGGUGCCCUUUCAUCAUGGCU | |
| | UUGGCAUGUUUACCACCCUGGGCUACCUCAUCUGU | |
| | GGCUUCAGGGUGGUGCUCAUGUAUCGCUUUGAGGA | |
| | AGAACUGUUUCUGCGGAGCCUGCAGGACUACAAGA | |
| | UCCAGAGCGCUCUGCUGGUGCCCACCCUGUUUAGC | |
| | UUCUUCGCCAAGAGCACCCUCAUUGAUAAGUAUGA | |
| | UCUGAGCAAUCUGCACGAGAUCGCCAGCGGCGGGG | |
| | CCCCCCUGAGCAAGGAGGUGGGAGAAGCCGUGGCC | |
| | AAACGGUUCCACCUCCCUGGGAUCAGGCAGGGCUA | |
| | UGGACUGACCGAAACCACCAGCGCCAUCCUGAUCA | |
| | CCCCCGAAGGCGAUGACAAGCCCGGCGCUGUGGGC | |
| | AAAGUGGUGCCCUUCUUCGAGGCUAAGGUGGUGGA | |
| | UCUGGACACUGGCAAAACCCUGGGGGUGAACCAGC | |
| | GGGGCGAGCUGUGCGUGCGGGGGCCUAUGAUUAUG | |
| | AGCGGCUACGUGAAUAACCCCGAGGCAACCAAUGC | |
| | CCUGAUCGACAAAGAUGGCUGGCUGCACUCUGGGG | |
| | ACAUUGCCUAUUGGGACGAAGACGAGCACUUCUUC | |
| | AUCGUCGAUCGGCUGAAGAGCCUGAUCAAGUAUAA | |
| | AGGCUACCAGGUGGCCCCCGCCGAACUGGAGAGCA | |
| | UUCUGCUGCAGCACCCCAACAUCUUUGAUGCCGGC | |
| 56 | GUGGCAGGCCUGCCAGAUGAUGACGCCGGCGAACU | eGFP-NLS |
| | GCCCGCCGCCGUGGUGGUGCUGGAGCACGGCAAGA | |
| | CCAUGACCGAGAAAGAAAUCGUGGACUAUGUGGCC | |
| | UCUCAGGUGACCACCGCCAAGAAGCUGCGGGGCGG | |
| | GGUGGUGUUCGUCGACGAGGUCCCAAAGGGCCUGA | |
| | CCGGCAAGCUGGAUGCCCGGAAAAUCCGGGAGAUC | |
| | CUGAUCAAGGCCAAGAAAGGCGGCAAGAUCGCCGU | |
| | GuaauggcggaaugguAGACGCUACGGACUU | |
| | AAAUAAUUGAGCCUUAGAGAAGAAAUUCUUUAAGU | |
| | GGAUGCUCUCAAACUCAGGGAAACCUAAAUCUAGC | |
| | UAGACACCAC | |
| | AUGGUGAGCAAAGGAGAAGAGCUAUUUACAGGCGU | |
| | GGUCCCCAUCCUCGUAGAACUGGAUGGAGACGUGA | |
| | ACGGCCACAAAUUUAGCGUCAGCGGGGAGGGCGAA | |
| | GGCGAUGCCACAUACGGCAAGCUGACUUUGAAGUU | |
| | UAUUUGUACCACUGGGAAACUGCCUGUGCCCUGGC | |
| | CCACCCUGGUCACCACACUGACCUAUGGGGUGCAG | |
| | UGCUUCUCCCGGUACCCCGAUCACAUGAAGCAGCA | |
| | CGACUUCUUCAAAUCCGCUAUGCCAGAAGGAUACG | |
| | UUCAAGAGCGAACUAUCUUCUUCAAGGAUGACGGU | |
| | AACUACAAGACAAGGGCCGAAGUCAAGUUUGAGGG | |
| | CGAUACACUAGUGAAUCGCAUUGAGCUGAAAGGCA | |
| | UAGACUUUAAAGAGGAUGGGAACAUCCUGGGGCAC | |
| | AAGCUCGAAUACAACUACAACUCCCACAAUGUGUA | |
| | CAUCAUGGCAGAUAAGCAGAAGAACGGUAUAAAGG | |
| | UGAACUUUAAGAUCAGACAUAACAUUGAGGAUGGU | |
| | UCAGUGCAACUCGCAGACCAUUACCAGCAAAACAC | |
| | ACCAAUCGGAGAUGGCCCCGUUCUGCUGCCCGAUA | |
| | AUCAUUACCUGAGUACACAGAGCGCCCUGUCAAAG | |
| | GACCCAAAUGAAAAACGUGAUCACAUGGUGCUCUU | |
| | AGAAUUUGUGACCGCCGCUGGGAUCACACUGGGCA | |
| | UGGAUGAGCUCUAUAAACGCAGUAGAGAUAUCUCC | |
| | CACGGCUUUCCUCCCGCCGUGGCAGCACAGGAUGA | |
| | UGGCACACUGCCGAUGUCCUGCGCUCAAGAGAGCG | |
| | GGAUGGAUCGGCAUCCCGCCGCUUGUGCUUCUGCG | |
| | CGGAUAAACGUAGGAUCCGCCGCUAAAAGGCCGGC | |
| | GGCCACGAAAAAGGCCGGCCAGGCAAAAAAGAAAA | |
| | AGCUGGACGGCAGCGGAAGCCCCAAGAAGAAGAGG | |
| | AAGGUAUAA | |
| 57 | AACAGAUAACUUACAGCUAGUCGGAAGGUGCAGAG | I-P-RNA- |
| | ACUCGACGGGAGCUACCCUAACGUCAAGACGAGGG | eGFP-NLS |
| | UAAAGAGAGAGUCCAAUUCUCAAAGCCAAUAGGCA | |
| | GUAGCGAAAGCUGCGGGAGAAUG | |
| | AAAAUCCGUUGACCUUAAACGGUCGUGUGGGUUCA | |
| | AGUCCCUCCACCCCCA | |
| | CCCCUCUCCCUCCCCCCCUAACGUUACUGGCCGAAG | |
| | CCGCUUGGAAUAAGGCCGGUGUGCGUUUGUCUAUA | |
| | UGUUAUUUUCCACCAUAUUGCCGUCUUUUGGCAAU | |
| | GUGAGGGCCCGGAAACCUGGCCCUGUCUUCUUGAC | |
| | GAGCAUUCCUAGGGGUCUUUCCCCUCUCGCCAAAG | |
| | GAAUGCAAGGUCUGUUGAAUGUCGUGAAGGAAGCA | |
| | GUUCCUCUGGAAGCUUCUUGAAGACAAACAACGUC | |
| | UGUAGCGACCCUUUGCAGGCAGCGGAACCCCCCACC | |
| | UGGCGACAGGUGCCUCUGCGGCCAAAAGCCACGUG | |
| | UAUAAGAUACACCUGCAAAGGCGGCACAACCCCAG | |
| | UGCCACGUUGUGAGUUGGAUAGUUGUGGAAAGAGU | |
| | CAAAUGGCUCUCCUCAAGCGUAUUCAACAAGGGGC | |
| | UGAAGGAUGCCCAGAAGGUACCCCAUUGUAUGGGA | |
| | UCUGAUCUGGGGCCUCGGUGCACAUGCUUUACAUG | |
| | UGUUUAGUCGAGGUUAAAAAACGUCUAGGCCCCCC | |
| | GAACCACGGGGACGUGGUUUUCCUUUGAAAAACAC | |
| | GAUGAUAAUAUGGCCACAACC | |
| | AUGGUGAGCAAAGGAGAAGAGCUAUUUACAGGCGU | |
| | GGUCCCCAUCCUCGUAGAACUGGAUGGAGACGUGA | |
| | ACGGCCACAAAUUUAGCGUCAGCGGGGAGGGCGAA | |
| | GGCGAUGCCACAUACGGCAAGCUGACUUUGAAGUU | |
| | UAUUUGUACCACUGGGAAACUGCCUGUGCCCUGGC | |
| | CCACCCUGGUCACCACACUGACCUAUGGGGUGCAG | |
| | UGCUUCUCCCGGUACCCCGAUCACAUGAAGCAGCA | |
| | CGACUUCUUCAAAUCCGCUAUGCCAGAAGGAUACG | |
| | UUCAAGAGCGAACUAUCUUCUUCAAGGAUGACGGU | |
| | AACUACAAGACAAGGGCCGAAGUCAAGUUUGAGGG | |
| | CGAUACACUAGUGAAUCGCAUUGAGCUGAAAGGCA | |
| | UAGACUUUAAAGAGGAUGGGAACAUCCUGGGGCAC | |
| | AAGCUCGAAUACAACUACAACUCCCACAAUGUGUA | |
| | CAUCAUGGCAGAUAAGCAGAAGAACGGUAUAAAGG | |
| | UGAACUUUAAGAUCAGACAUAACAUUGAGGAUGGU | |
| | UCAGUGCAACUCGCAGACCAUUACCAGCAAAACAC | |
| | ACCAAUCGGAGAUGGCCCCGUUCUGCUGCCCGAUA | |
| | AUCAUUACCUGAGUACACAGAGCGCCCUGUCAAAG | |
| | GACCCAAAUGAAAAACGUGAUCACAUGGUGCUCUU | |
| | AGAAUUUGUGACCGCCGCUGGGAUCACACUGGGCA | |
| | UGGAUGAGCUCUAUAAACGCAGUAGAGAUAUCUCC | |
| | CACGGCUUUCCUCCCGCCGUGGCAGCACAGGAUGA | |
| | UGGCACACUGCCGAUGUCCUGCGCUCAAGAGAGCG | |
| | GGAUGGAUCGGCAUCCCGCCGCUUGUGCUUCUGCG | |
| | CGGAUAAACGUAGGAUCCGCCGCUAAAAGGCCGGC | |
| | GGCCACGAAAAAGGCCGGCCAGGCAAAAAAGAAAA | |
| | AGCUGGACGGCAGCGGAAGCCCCAAGAAGAAGAGG | |
| | AAGGUAUAAuggcggaaugguAGACGCUACGGACUU | |
| | AAAUAAUUGAGCCUUAGAGAAGAAAUUCUUUAAGU | |
| | GGAUGCUCUCAAACUCAGGGAAACCUAAAUCUAGC | |
| | UAGACACCAC | |
| 58 | AGACGCUACGGACUUAAAUAAUUGAGCCUUAGAGA | Group I |
| | AGAAAUUCUUUAAGUGGAUGCUCUCAAACUCAGGG | intron from |
| | AAACCUAAAUCUAGCUAUAGACAAGGCAAUCCUGA | Anabaena |
| | GCCAAGCCGAAGUAGUAAUUAGUAAGUUAACAACA | pre-tRNA |
| | GAUAACUUACAGCUAGUCGGAAGGUGCAGAGACUC | |
| | GACGGGAGCUACCCUAACGUCAAGACGAGGGUAAA | |
| | GAGAGAGUCCAAUUCUCAAAGCCAAUAGGCAGUAG | |
| | CGAAAGCUGCGGGAGAAUGAAAAUCCGUUGACCUU | |
| | AAACGGUCGUGUGGGUUCAAGUCCCUCCACCCCCA | |
| 59 | ACGGACUU | 5′ exon |
| 60 | AAAAUCCGU | 3′ exon |

1. A pair of RNA molecules for making a circular RNA, comprising:
(1) a first RNA molecule comprising the following elements operably connected and arranged in the order from the 5′-end to the 3′-end of the molecule:
i) a 3′ intron sequence of a Group I intron,
ii) a 3′ splice site of the Group I intron,
iii) optionally, a 3′ exon sequence of the Group I intron,
iv) an RNA sequence of interest,
v) optionally, a 5′ exon sequence of the Group I intron,
vi) a 5′ splice site of the Group I intron, and
vii) a 5′ intron sequence of the Group I intron, and
(2) a second RNA molecule comprising an intermediate intron sequence of the Group I intron,
wherein when the first RNA molecule is contacted with the second RNA molecule, a scaffolding domain and a catalytic domain of the Group I intron are formed to thereby allow the production of the circular RNA comprising the 3′ exon sequence, the RNA sequence of interest and the 5′ exon sequence; and
wherein the 3′ intron sequence comprises the R sequence and the S sequence of the Group I intron, the 5′ intron sequence comprises the internal guide sequence (IGS) and the P sequence of the Group I intron, and the intermediate intron sequence comprises the Q sequence of the Group I intron.

2. (canceled)

3. The pair of RNA molecules of claim 1, wherein the first RNA molecule comprises, from the 5′-end to the 3′-end of the molecule,
a) Fragment 3 comprising the 3′ intron sequence and the 3′ splice site operably connected and arranged in the order from the 5′-end to the 3′-end of Fragment 3;
b) optionally, the 3′ exon sequence;
c) the RNA sequence of interest;
d) optionally, the 5′ exon sequence;
e) Fragment 1 comprising the 5′ splice site and the 5′ intron sequence operably connected and arranged in the order from the 5′-end to the 3′-end of Fragment 1; and
the second RNA molecule comprises Fragment 2 having the intermediate intron sequence.

4. (canceled)

5. The pair of RNA molecules of claim 3, wherein the three portions of the Group I intron are obtained by dividing the intron with an interrupted site located within a loop of the P6 stem-loop region of the Group I intron, and another interrupted site located within a loop of the P2, P5, P8, or P9 stem-loop region of the Group I intron.

6.-10. (canceled)

11. The pair of RNA molecules of claim 3, wherein Fragment 1 comprises a nucleotide sequence that is at least 80% identical, such as at least 80%, 85%, 90%, 91%, 92%, 93%, 94%, 95%, 96%, 97%, 98%, 99% or 100% identical, to the nucleotide sequence of SEQ ID NO: 11, 14, 17 or 20.

12. The pair of RNA molecules of claim 3, wherein Fragment 2 comprises a nucleotide sequence that is at least 80% identical, such as at least 80%, 85%, 90%, 91%, 92%, 93%, 94%, 95%, 96%, 97%, 98%, 99% or 100% identical, to the nucleotide sequence of SEQ ID NO: 12, 15, 18, 21, 23, 25, 27 or 29.

13. The pair of RNA molecules of claim 3, wherein Fragment 3 comprises a nucleotide sequence that is at least 80% identical, such as at least 80%, 85%, 90%, 91%, 92%, 93%, 94%, 95%, 96%, 97%, 98%, 99% or 100% identical, to the nucleotide sequence of SEQ ID NO: 13, 16, 19, 22, 24, 26, 28 or 30.

14. The pair of RNA molecules of claim 3, wherein Fragment 1, Fragment 2 and Fragment 3 comprise the nucleotide sequence of:
a. SEQ ID NO: 11, SEQ ID NO:12, and SEQ ID NO: 13, respectively;
b. SEQ ID NO: 14, SEQ ID NO:15, and SEQ ID NO: 16, respectively;
c. SEQ ID NO: 17, SEQ ID NO: 18, and SEQ ID NO: 19, respectively;
d. SEQ ID NO: 20, SEQ ID NO: 21, and SEQ ID NO: 22, respectively;
e. SEQ ID NO: 14, SEQ ID NO: 23, and SEQ ID NO: 24, respectively;
f. SEQ ID NO: 14, SEQ ID NO: 25, and SEQ ID NO: 26, respectively;
g. SEQ ID NO: 14, SEQ ID NO: 27, and SEQ ID NO: 28, respectively; or
h. SEQ ID NO: 14, SEQ ID NO: 29, and SEQ ID NO: 30, respectively.

15. The pair of RNA molecules of claim 14, wherein the first RNA molecule comprises the 3′ exon sequence.

16. The pair of RNA molecules of claim 15, wherein the first RNA molecule comprises the 5′ exon sequence.

17. The pair of RNA molecules of claim 16, wherein the 3′ exon sequence is at least 95%, at least 96%, at least 97% at least 99% or at least 99% identical to the full length sequence of the native 3′ exon sequence.

18. (canceled)

19. The pair of RNA molecules of claim 16, wherein the 5′ exon sequence is at least 95%, at least 96%, at least 97%, at least 98%, or at least 99% identical to a contiguous fragment starting from the 3′ terminal nucleotide of the native 5′ exon sequence.

20. (canceled)

21. The pair of RNA molecules of claim 16, wherein the 3′ exon sequence consists of SEQ ID NO: 60 or SEQ ID NO: 3; and the 5′ sequence consists of SEQ ID NO: 59 or SEQ ID NO: 4.

22. The pair of RNA molecules of claim 21, wherein the nucleotide sequence of interest comprises at least one protein-coding sequence and an internal ribosome entry site (IRES) operably linked thereto.

23. (canceled)

24. The pair of RNA molecules of claim 22, wherein the IRES comprises or consists of the nucleotide sequence of SEQ ID NO: 51.

25. The pair of RNA molecules of claim 24, wherein the nucleotide sequence of interest encodes a protein, or the nucleotide sequence of interest is a non-protein coding sequence.

26.-35. (canceled)

36. The pair of RNA molecules of claim 25, wherein the first RNA molecule further comprises a 3′-end binding motif, and the second RNA molecule further comprises a 5′-end binding motif, wherein the 3′-end binding motif and the 5′-end binding motif are capable of complementary pairing to form a double-stranded region, preferably the 3′-end binding motif and the 5′-end binding motif are perfectly complimentary to each other.

37. The pair of RNA molecules of claim 36, wherein the 3′-end binding motif or 5′-end binding motif is about 5-50, about 10-50, about 20-50, about 30-50, about 40-50, about 5-40, about 5-30, about 5-20, or about 10-20 nucleotides in length; preferably about 10, 15, or 19 nucleotides in length.

38.-50. (canceled)
